(19) United States
(12) Patent Application Publication
(10) Pub. No.: US 2026/0053648 A1
(43) Pub. Date: Feb. 26, 2026

(54) DEVICES, SYSTEMS, AND METHODS FOR TREATING AIRWAY COLLAPSE

(71) Applicant: Apreo Health, Inc., Menlo Park, CA (US)

(72) Inventors: Jagannath Padmanabhan, San Carlos, CA (US); Brandon Wai-lon Chu, San Francisco, CA (US); Mark Stuart Leung, Duncan (CA); Patrick P. Wu, San Carlos, CA (US); Allison Sihan Jia, San Francisco, CA (US); Claudia Lynn Pham, San Francisco, CA (US); Hanson S. Gifford, III, Woodside, CA (US); Martin L. Mayse, Wayzata, MN (US); Karun D. Naga, Los Altos, CA (US)

(21) Appl. No.: 19/279,967

(22) Filed: Jul. 24, 2025

Related U.S. Application Data
(63) Continuation of application No. PCT/US2024/013013, filed on Jan. 25, 2024.
(60) Provisional application No. 63/441,172, filed on Jan. 25, 2023.

(51) Int. Cl. A61F 2/86

(52) U.S. Cl. CPC A61F 2/86; A61F 2230/0004; A61F 2230/0034; A61F 2250/0018

(57) ABSTRACT

The present technology includes an implant for treating excessive central airway collapse (ECAC) of an airway. The implant can comprise a wire-form stent configured to be positioned within the airway. The stent can comprise a C-shaped sidewall, and is configured to be positioned within the airway such that the C-shaped sidewall is positioned proximate a cartilaginous portion of the airway and presses outwardly against the cartilaginous portion.

### CROSS-REFERENCE TO RELATED APPLICATION(S)

The present application is a continuation of International Application No. PCT/US2024/013013, filed Jan. 25, 2024, which claims the benefit of priority to U.S. Provisional Application No. 63/441,172, titled DEVICES, SYSTEMS, AND METHODS FOR TREATING AIRWAY COLLAPSE, filed Jan. 25, 2023, each of which is incorporated by reference herein in its entirety.

The present application incorporates by reference the following applications, in their entireties: PCT Application No. TBD [Attorney Docket No. APH. 005WO], titled ROBOTIC SYSTEMS FOR DELIVERING ENDOBRONCHIAL IMPLANTS AND RELATED TECHNOLOGY, filed concurrently herewith, PCT Application No. TBD [Attorney Docket No. APH. 007WO], titled METHODS AND SYSTEMS FOR TREATING PULMONARY DISEASE, filed concurrently herewith, and PCT Application No. PCT/US22/73962, ENDOBRONCHIAL IMPLANTS AND RELATED TECHNOLOGIES, filed Jul. 20, 2022.

### TECHNICAL FIELD

The present technology relates to devices, systems, and methods for treating airway collapse. In particular, the present technology relates to devices, systems, and methods for treating excessive central airway collapse.

### BACKGROUND

Excessive central airway collapse (ECAC) is a congenital or acquired deficiency of tracheal and/or bronchial cartilages. ECAC may be characterized by flaccidity of the supporting tracheal or bronchial cartilage, resulting in airway obstruction, respiratory difficulties and, in severe cases, death. Referring to FIG. 1, excessive dynamic airway collapse (EDAC) and tracheobronchomalacia (TBM) are two recognized pathophysiologic entities associated with ECAC, although the clinical distinction between the two conditions is often unclear. EDAC is typically characterized by excessive forward displacement of the smooth muscle posterior membrane due to weakness and atrophy of the longitudinal elastic fibers of the posterior wall. TBM is often characterized as one of two morphological types. Cartilaginous type TBM refers to weakness of the lateral and anterior cartilaginous walls of the airways. This type can have a crescent or saber-sheath appearance depending on whether the anterior or lateral walls of the airway are weakened. Circumferential or concentric type TBM is characterized by anterior and lateral airway wall collapse and is usually associated with inflammatory conditions such as relapsing polychondritis. Examples of a normal trachea and a diseased trachea during inspiration and expiration are shown in FIG. 2.

ECAC presents with respiratory difficulties, chronic cough, wheezing, recurrent infections, and in severe cases, acute life-threatening events. In adults and infants who have severe forms of ECAC that are unresponsive to medical management or have life-threatening symptoms, surgical intervention may be necessary. Severe cases can result in death or require tracheostomy with ventilation for 2-3 years. Current treatment techniques often include mechanical ventilation, implantation of tracheal stents, and surgery. Unfortunately, outcomes of all surgical/mechanical interventions to date have been widely associated with failure, morbidity, and mortality.

Surgical/mechanical devices for addressing ECAC can be broadly categorized into stents, namely devices placed inside or within the passageway (e.g., trachea), and splints, namely devices implanted externally around the passageway (e.g., the trachea). Stents offer an easier surgical approach, but are associated with more complications, including stent migration, granulation tissue leading to secondary obstruction, the need for multiple procedures and even death. Further, conventional silicone and metal tracheal stents have posed difficulties in placement, stent migration or distortion, lack of tissue growth, permanence, and decreased adjustability. These complications have prompted the U.S. Food and Drug Administration (FDA) to issue a warning against the use of tracheal stents.

Splints, however, have also exhibited various complications, including tracheal erosion and granuloma formation. These detrimental effects are believed to be attributable to the conventional materials used to form the splints in that they are too stiff. Further, there is concern that overly stiff devices restrict tracheobronchial growth, causing more long-term complications, especially in children. Tracheal banding in rat models has been found not only to restrict tracheal growth, but to lead to smaller overall lung volumes, less alveoli and smaller overall body growth. However, devices that are not stiff enough fail to create/maintain tracheobronchial patency and easily migrate from the desired position. While degradable devices have been proposed as a partial solution, these devices have been observed to degrade too rapidly, thus allowing insufficient time for the trachea to remodel and become structurally competent, thereby failing to resolve the ECAC or other passageway defects. Thus, implantable devices that improve upon these issues to decrease mortality, improve patient outcomes, including improve growth to permit the defective passageway to develop structural competency, and to enhance the quality of life of a patient having such a passageway defect would be desirable.

### SUMMARY

The subject technology is illustrated, for example, according to various aspects described below, including with reference to FIGS. 1-63C. Various examples of aspects of the subject technology are described as numbered examples (1, 2, 3, etc.) for convenience. These are provided as examples and do not limit the subject technology.

1. A method of treating a patient with tracheobronchomalacia and/or excessive dynamic airway collapse, the method comprising:
positioning a device within a lumen of an airway of the patient such that at least a portion of the device engages a cartilaginous portion of a sidewall surrounding the lumen to prevent a cross-sectional dimension of the lumen from decreasing to less than 50% of a maximum cross-sectional dimension of the lumen.

2. The method of Example 1, wherein the airway is a trachea.

3. The method of Example 1 or Example 2, wherein the airway is a primary bronchus.

4. The method of any one of Examples 1 to 3, wherein the device maintains the cross-sectional dimension of the lumen within a range of about 50% to about 100% of the maximum cross-sectional dimension of the lumen.

5. The method of any one of Examples 1 to 4, wherein the device permits the airway to longitudinally lengthen and shorten while the device is positioned within the lumen.

6. The method of any one of Examples 1 to 5, wherein the sidewall comprises a muscular portion.

7. The method of Example 6, wherein the muscular portion comprises smooth muscle.

8. The method of Example 6 or Example 7, wherein the muscular portion comprises a trachealis muscle.

9. The method of any one of Examples 6 to 8, wherein the device permits contraction and expansion of the muscular portion.

10. The method of any one of Examples 1 to 9, wherein the device prevents contraction and expansion of the cartilaginous section.

11. The method of any one of Examples 1 to 10, wherein the device has a proximal portion, a distal portion, and an intermediate portion therebetween.

12. The method of Example 11, wherein the proximal portion is more flexible than the intermediate portion.

13. The method of Example 11 or Example 12, wherein the distal portion is more flexible than the intermediate portion.

14. The method of any one of Examples 1 to 13, wherein a surface area of the portion of the device engaging the cartilaginous portion of the sidewall is less than 10% of a total surface area of the sidewall.

15. The method of any one of Examples 1 to 14, wherein a surface area of a portion of the device engaging the sidewall is less than 10% of a total surface area of the sidewall.

16. The method of any one of Examples 6 to 15, wherein a surface area of a portion of the device engaging the muscular portion of the sidewall is at least 50% of a total surface area of the sidewall.

17. The method of any one of Examples 1 to 16, wherein the device is formed from an elongate member.

18. The method of any one of Examples 1 to 17, wherein the device is formed from a single elongate member.

19. The method of Example 17 or Example 18, wherein the elongate member comprises nitinol.

20 The method of Example 19, wherein the elongate member has an austenite finish temperature between about 5 degrees Celsius to about 30 degrees Celsius.

21. The method of Example 20, wherein the austenite finish temperature is between about 8 degrees Celsius and about 12 degrees Celsius.

22. The method of any one of Examples 17 to 21, wherein the elongate member has a diameter of about 0.009 inches to about 0.030 inches.

23 The method of any one of Examples 17 to 22, wherein the elongate member has a consistent diameter along its length.

24. The method of any one of Examples 17 to 23, wherein the elongate member has a proximal portion, a distal portion, and an intermediate portion therebetween, and wherein a diameter of the elongate member at the proximal portion and/or the distal portion is less than the diameter of the elongate member at the intermediate portion.

25 The method of any one of Examples 17 to 24, wherein the elongate member forms a plurality of bends, each bend of the plurality of bends having a radius of curvature no less than about 2.0 millimeters.

26. The method of Example 25, wherein each bend of the plurality of bends has a radius of curvature no less than about 0.6 millimeters.

27. The method of any one of Examples 1 to 26, further comprising removing the device from the lumen of the airway.

28. The method of any one of Examples 17 to 26, further comprising removing the device from the lumen of the airway, and wherein removing the device comprises applying a proximally directed force to a terminus of the elongate member.

29. The method of any one of Examples 6 to 28, wherein the device includes a tether, and wherein the tether is configured to extend across the muscular portion of the sidewall.

30. The method of one of Examples 17 to 28, wherein the device includes a tether, and wherein the elongate member is configured to engage the cartilaginous portion of the sidewall and the tether is configured to extend across the muscular portion of the sidewall.

31. The method of Example 29 or Example 30, wherein the tether comprises Kevlar thread.

32. The method of any one of Examples 6 to 31, wherein the device comprises an elongated rod configured to be positioned at a location within the lumen proximate a junction between the cartilaginous portion of the sidewall and the muscular portion of the sidewall.

33 The method of Example 32, wherein the elongated rod is configured to extend substantially parallel to a longitudinal dimension of the airway when the device is implanted in the lumen.

34. The method of Example 32 or Example 33, wherein the elongated rod is flexible.

35. The method of any one of Examples 32 to 34, wherein the device comprises at least two elongated rods.

36. The method of Example 35, wherein a support wire extends between and connects the at least two elongated rods to one another.

37. The method of any one of Examples 1 to 36, wherein positioning the device within the lumen comprises advancing the device through a lumen of a delivery sheath.

38 The method of Example 37, wherein advancing the device through the delivery sheath lumen comprises advancing a rod positioned within the delivery sheath lumen.

39. The method of Example 38, wherein the rod engages a distal end of the device.

40. The method of any one of Examples 1 to 39, wherein positioning the device within the lumen comprises maintaining a position of a distal end of the device relative to the airway while retracting the delivery sheath relative to the device to allow the device to expand.

41. The method of any one of Examples 1 to 40, wherein positioning the device within the lumen comprises positioning a first portion of the device within a lumen of a trachea of the patient, positioning a second portion of the device within a lumen of a right primary bronchus of the patient, and positioning a third portion of the device within a lumen of a left primary bronchus of the patient.

42. The method of Example 41, wherein the first portion, the second portion, and the third portion of the device are connected to one another at a Y-junction.

43 The method of Example 42, wherein the Y-junction is made from silicone.

44. The method of Example 42 or Example 43, wherein the Y-junction comprises a first lumen branching into a second and third lumens.

45. The method of Example 44, wherein the first lumen is continuous with a lumen in the first portion, the second lumen is continuous with a lumen in the second portion, and the third lumen is continuous with the third portion.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 shows a cross-section of a normal trachea in comparison to cross-sections of tracheas exhibiting EDAC and TBM.

FIG. 2 shows examples of a normal trachea and a diseased trachea during inspiration and expiration.

FIGS. 3 and 4 show various anatomical features of the trachea and main bronchi.

FIGS. 5-9 show implants configured to treat ECAC in accordance with the present technology.

FIG. 10 is a delivery device configured in accordance with the present technology.

FIG. 11 shows a method for delivering an implant of the present technology to a patient's trachea.

FIG. 12 shows an implant of the present technology implanted within a patient's trachea.

FIG. 13 shows a plurality of implants configured in accordance with the present technology, each positioned within one of a patient's trachea, left main bronchus, and right main bronchus.

FIGS. 14-16 show various implants configured in accordance with the present technology.

FIGS. 17A and 17B depict a delivery system configured in accordance with the present technology.

FIGS. 18-31 show various implants configured in accordance with the present technology.

FIGS. 32-35 show a method of deploying an implant configured in accordance with the present technology.

FIG. 36 is flow diagram showing a method of deploying an implant configured in accordance with the present technology.

FIGS. 37-61B show various implants configured in accordance with the present technology.

FIG. 62 shows a delivery system configured in accordance with the present technology, shown while delivery an implant to a trachea.

FIGS. 63A-63C are CT scans showing a patient's trachea before implantation of an implant in a peripheral airway and 90 days after implantation.

### DETAILED DESCRIPTION

The present technology is directed to implants configured to be positioned in the trachea and/or main bronchi (referred to herein as “proximal airways”) to establish and maintain airway patency, in particular to treat patients diagnosed with ECAC (including patients diagnosed with TBM and/or EDAC). Implants in accordance with at least some embodiments of the present technology are configured to be intraluminally positioned within a proximal airway and expanded against the proximal airway wall, thereby distending and/or dilating the proximal airway and increasing the cross-sectional area of the proximal airway lumen. In at least some cases, the implants are configured to enlarge the proximal airway beyond its normal size.

Implants in accordance with embodiments of the present technology are configured to have relatively little (e.g., minimal) surface area contact with a proximal airway wall while maintaining stable contact with the wall during respiration. These and other features disclosed herein may reduce or eliminate the gradual airway occlusion by biological processes (e.g., inflammation, fibrosis, granulation, mucous impaction, etc.) that would otherwise limit the effectiveness of implants for the treatment of ECAC.

Treatment within a proximal airway may involve using robotic assistance to place an implant in a specific location or it may involve placement of at least one implant in multiple locations (e.g., the trachea, left main bronchus, or right main bronchus). Determination of which parts of the proximal airways to treat can be made by the clinical operator (e.g., pulmonologist or surgeon) with the assistance of imaging (e.g., CT, ultrasound, radiography, or bronchoscopy) to assess the presence and pathology of disease and impact on pulmonary function and airflow dynamics.

Many specific details of devices, systems, and methods in accordance with various embodiments of the present technology are disclosed herein. Although these devices, systems, and methods may be disclosed primarily or entirely in the context of treating ECAC (sometimes TBM or EDAC in particular) and/or proximal airways, other contexts in addition to those disclosed herein are within the scope of the present technology. For example, suitable features of described devices, systems, and methods can be implemented in the context of treating chronic obstructive pulmonary disease (COPD) (including emphysema) or benign prostatic hyperplasia (BPH) among other examples. Any of the devices, systems, and methods herein can also be positioned within the more distal airways, i.e., branches distal to the main bronchi. Furthermore, it should be understood in general that other devices, systems, and methods in addition to those disclosed herein are within the scope of the present technology. For example, devices, systems, and methods in accordance with embodiments of the present technology can have different and/or additional configurations, components, and procedures than those disclosed herein. Moreover, a person of ordinary skill in the art will understand that devices, systems, and methods in accordance with embodiments of the present technology can be without one or more of the configurations, components, and/or procedures disclosed herein without deviating from the present technology.

### I. ANATOMY AND PHYSIOLOGY

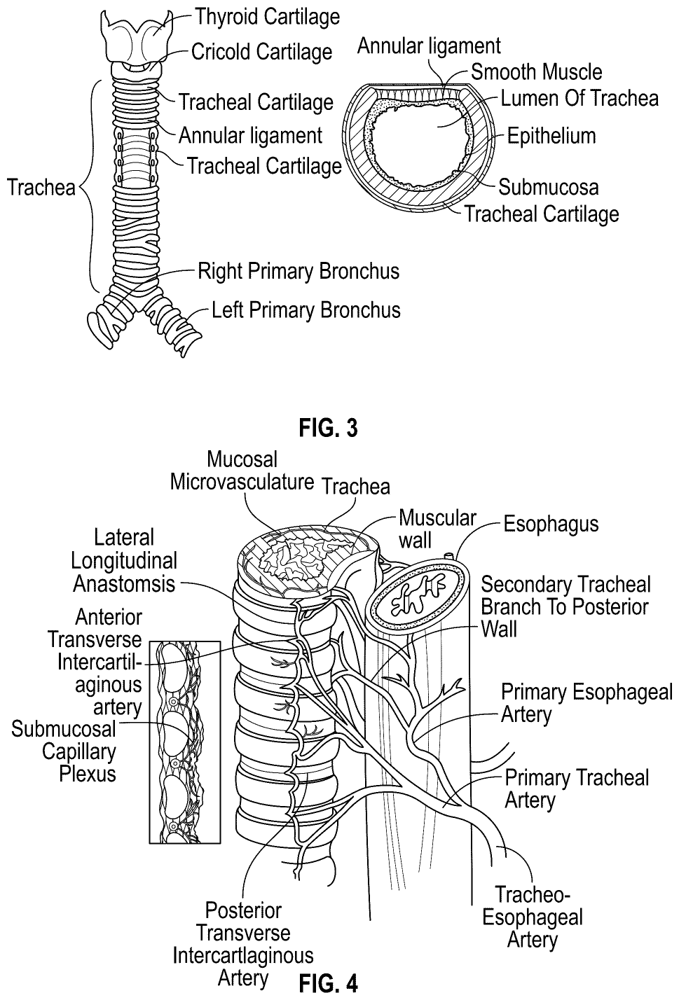

FIG. 3 is a schematic illustration of a portion of a tracheobronchial tree of a human subject. As shown in FIG. 3, the tracheobronchial tree includes a trachea that extends downwardly from the nose and mouth and divides into a left main bronchus and a right main bronchus. The left main bronchus and the right main bronchus each branch to form lobar bronchi, segmental bronchi, and sub-segmental bronchi, which have successively smaller diameters and shorter lengths as they extend distally. Both the trachea and proximal portions of the bronchial tree are surrounded by a plurality of cartilaginous rings that maintain patency of these airways despite pressure changes.

FIG. 4 is a perspective view of a portion of the trachea and an adjacent portion of the esophagus. The trachea is a D-shaped fibrocartilaginous passage comprising 16-22 tracheal cartilaginous rings anterolaterally and a fibromuscular wall posteriorly. The tracheal cartilaginous rings are composed of hyaline cartilage and interconnected by fibroelastic tissue. The posterior wall of the trachea is formed by the trachealis muscle, making the cartilaginous rings appear as incomplete, C-shaped rings. The structure of the trachealis muscular wall makes the trachea sufficiently flexible and elastic to permit the transient expansion of the esophagus during swallowing.

The trachea consists of four histological layers: the inner mucosal layer, the submucosal layer, the cartilage and muscle layer, and the outer adventitial layer. The innermost layer, the mucosal layer, is lined with pseudostratified ciliated columnar epithelium. Radially inward of the mucosal layer is the submucosal layer. The submucosal layer consists of connective tissue that contains mucus glands, smooth muscle, vessels, nerves, and lymphatics. Radially inward of the submucosal layer is the musculocartilaginous layer, which comprises the cartilaginous rings and intervening smooth muscle. Lastly, the most external layer comprises fibroelastic adventitia. The average adult trachea is about 1.8 cm in diameter and about 10-12 cm in length. The neonatal trachea is about 3-4.5 mm in diameter and about 20-30 mm in length.

### II. SELECTED EXAMPLES OF IMPLANTS FOR TREATING ECAC

In some of the embodiments described herein, it may be advantageous for the expandable device to modify and/or alter the airway wall. In one example, the expandable device comprises self-expanding capabilities (e.g., nitinol construction), whereby deployment of the expandable device results in the application of a chronic outward force to the airway wall that causes a gradual dilation of the airway wall and expansion of the airway lumen. In this example, the self-expansion of the expandable device would cause the airway wall to expand beyond its native diameter. Additionally, or alternatively, expansion of the expandable device can be facilitated by a balloon configured to be inflated to force expansion of the expandable device. Forced expansion of the expandable device via a balloon (incorporated as part of a delivery system or separate from the delivery system) may be advantageous because the size and pressure of the balloon can be adjusted to control the expansion of the expandable device.

Controlled expansion of the expandable device is desirable in that such controlled expansion will allow for controlled modification of the airway wall. In one example, it may be desirable to cause dilation of the airway wall to increase the cross-sectional area of the airway lumen, but without creating substantial injury to the airway wall. An increase in the cross-sectional area would improve expiratory outflow, thereby yielding a therapeutic benefit in patients with ECAC.

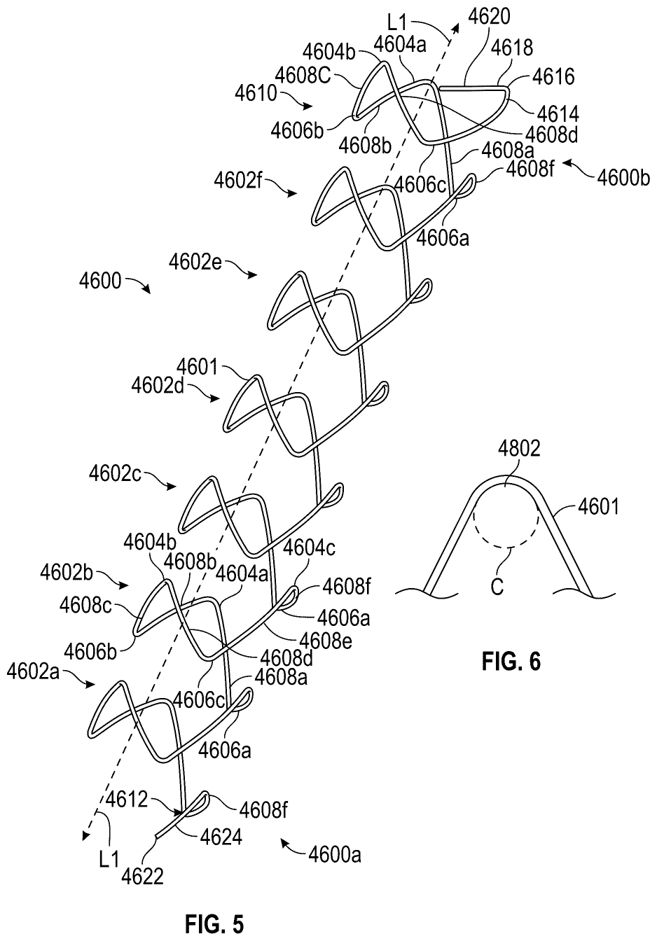

FIG. 5 is a perspective view of an expandable device 4600 configured in accordance with several embodiments of the present technology. In FIG. 5, the device 4600 is shown in an expanded, unconstrained state. The device 4600 has a proximal end portion 4600a, a distal end portion 4600b, and a longitudinal axis LI extending between the distal and proximal end portions 4600a, 4600b. The device 4600 can comprise a generally tubular structure formed of a wire 4601 wrapped around a longitudinal axis to form a series of bands 4602 (individually labeled as 4602a-4602f), each comprising a 360 degree turn of the wire 4601. The device 4600 further includes a distal structure 4610 distal of the distalmost band 4602f, and a proximal structure 4612 proximal of the proximalmost band 4602a. The wire 4601 undulates between the ends of a given band 4602 such that each band 4602 has a plurality of alternating peaks 4604 (individually labeled as 4604a-4604c) and valleys 4606 (individually labeled as 4606a-4606c) that are connected by struts 4608 (individually labeled as 4608a-4608f). The peaks 4604 can comprise the bend apices within a given band 4602 that are closer to and/or point towards the second end portion 4600b of the device 4600, and the valleys 4606 can comprise the bend apices within a given band 4602 that are closer to and/or point towards the first end portion 4600b of the device 4600. The serpentine configuration of each turn of the wire 4601 makes it easier to radially compress the device 4600 onto and/or into a delivery system, and easier to accurately deploy the device 4600, as discussed in greater detail below.

Each band 4602 can have first, second, and third peaks 4604a, 4604b, and 4604c, first, second, and third valleys 4606a, 4606b, and 4606c, and first, second, third, fourth, fifth, and sixth struts 4608a, 4608b, 4608c, 4608d, 4608e, and 4608f. The bands 4602 are connected end-to-end such that each band 4602 begins at a first valley 4606a and ends where the sixth strut 4608f meets the first valley 4606a of the next band 4602 (or, in the case of the sixth band 4602f, where the sixth strut 4608f meets the first valley 4606a of the distal structure 4610). Starting at a first valley 4606a and moving distally in a clockwise direction, each band 4602 has a first strut 4608a extending distally from the first valley 4606a to a first peak 4604a, then a second strut 4608b extending proximally from the first peak 4604a to a second valley 4606b, then a third strut 4608c extending distally from the second valley 4606b to a second peak 4604b, then a fourth strut 4608d extending proximally from the second peak 4604b to a third valley 4606c, then a fifth strut 4608c extending distally from the third valley 4606c to a third peak 4604c, then a sixth strut 4608f extending proximally from the third peak 4604 until terminating at the first valley 4606a of the next band 4602. While the device 4600 shown in FIG. 5 comprises three peaks and three valleys per turn, in other embodiments the device 4600 can have any number of peaks and valleys per turn. Moreover, while all of the bands 4602 have the same number of peaks and valleys, in other embodiments some or all of the bands 4602 within the same device can have different numbers of peaks and valleys.

Along the length of the device 4600, and within a given band 4602, the wire 4601 has struts 4608 that extend both proximally and distally in the direction of the wire turn. For example, following the wire 4601 in a clockwise direction around the turn, the device 4601 has struts 4608 that extend distally, then proximally, then distally, then proximally, then distally, thereby forming a plurality of localized, V-shaped braces that when placed within an airway support the airway wall and serve to tent open the airway lumen. This is in contrast to a simple coil in which the wire extends distally continuously as it wraps around each turn. In some embodiments, for example as shown in FIG. 5, the individual first and fifth struts 4608a and 4608c can be longer than the individual second, third, fourth, and sixth struts 4608b, 4608c, 4608d, and 4608f. In other embodiments the struts 4608 can have different lengths or configurations. Strut length can be measured along the longitudinal axis of the wire 4601. Likewise, the individual second, third, and fourth struts 4608b, 4608c, and 4608d can be longer than the sixth strut 4608f. In some embodiments, the length of the struts 4608 can be determined by the equation 3a-3b=1/pitch, where ‘a’ is the longer strut and ‘b’ is the shorter strut 4608.

As previously mentioned, the bands 4602 are connected to one another only by way of the single, continuous wire. Advantageously, all of the peaks 4604 and valleys 4606 are free peaks and valleys, meaning that none of the peaks 4604 and valleys 4606 are connected to a peak, valley, or other portion of a longitudinally adjacent band 4602. This lack of interconnectedness amongst axially adjacent structures provides the device 4600 with enhanced axial flexibility and stretchability as compared to conventional stents that include one or more bridges or other linkages between longitudinally adjacent struts and/or apices. This flexible configuration enables the device 4600 to stretch and bend with the airway in response to different loads (e.g., bending, torsion, tensile) associated with various anatomical conditions (e.g., airway bifurcation, curvature, etc.) and physiological conditions (e.g., respiration, coughing, etc.), thereby allowing the device to move with the airway to minimize relative motion while still maintaining a threshold radial force. In some embodiments, the device 4600 has a ratio of radial force to longitudinal stiffness that is greater than that of conventional stents. This longitudinal and bending flexibility to move with the airway also has the benefit of limiting relative motion between the device 4600 and the airway wall during respiration and other movements like coughing. Relative motion of the device 4600 to the airway wall can cause inflammation and formation of granulation tissue, which over time can partially or completely occlude the newly-opened lumen, thereby obstructing airflow and frustrating the purpose of treatment. Without being bound by theory, the elimination of longitudinal linkages and/or closed cells along the length of the device 4600 may help maintain perfusion of the treated portion of the airway wall, as closed cells can impede blood flow.

As described herein, there are several aspects of the device that contribute to minimizing granulation tissue formation. One aspect is the self-expanding structure and oversizing relative to the airway diameter that produces a chronic outward force against the airway wall that facilitates wall engagement and apposition, thereby minimizing relative motion. A second aspect is the lack of interconnectedness from the free peaks and valleys that allows for considerable flexibility, thereby allowing the device to move with the airway and minimize relative motion. A third aspect is the low material density and high porosity that cause lesser surface area contact with the airway wall, thereby producing less tissue reaction. A fourth aspect is the wire pattern having no closed cells so as to maintain perfusion, thereby minimizing tissue necrosis and local inflammatory reaction.

Another benefit of the lack of interconnectedness associated with the free peaks and valleys of the expandable device is the low tensile force required to disengage the device from the airway wall. A tensile axial load (i.e., pulling) applied to the wire will cause elongation that reduces the diameter of each loop or band, thereby moving each loop or band away from the airway wall. This separation from the airway wall can facilitate retrievability of the device following implantation with minimal trauma or disturbance to the airway wall.

One historical challenge with conventional, catheter-delivered implants (e.g., stents, braided structures) is the foreshortening that occurs during deployment and implantation. Such foreshortening can make it challenging to accurately deliver the implant to the intended treatment location. Foreshortening is often the result of elongation of the implant during radial compression into a reduced profile for minimally-invasive delivery. Elongation results from the implant's structural design and high material density (i.e., due to the structure and amount of material, the implant cannot stay in the same axial plane when radially compressed). In the device described herein, the lack of longitudinal bridges between axially adjacent structures and relatively low material density (as described below) results in radially compression to a delivery configuration with little to no elongation (e.g., 0%, 5% or less, 10% or less), thereby enabling the device 4600 to be deployed with little to no change in length. Thus, unlike braids and certain stents, the device 4600 does not experience foreshortening when radially expanding. The length of the 4600 device in a compressed, delivery state (for example, see FIG. 8) is substantially the same as the length of the device 4600 in an expanded, unconstrained state. As a result, the device 4600 can be deployed more predictably and with greater landing accuracy.

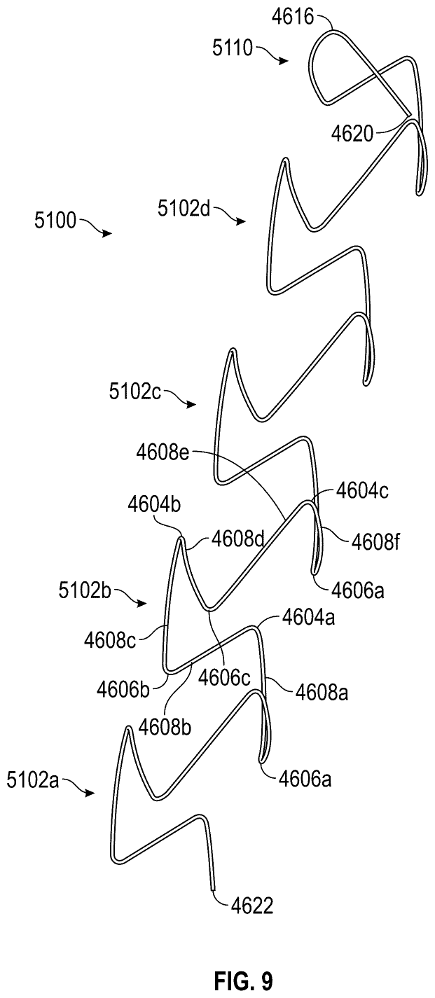

As shown in FIG. 5, the device 4600 can have a turn density that is measured by the number of full (i.e., 360 degree) turns along an inch of the device 4600. It can be advantageous to have a turn density that is low enough (e.g., adjacent turns are longitudinally farther apart) to allow for sufficient spacing between the adjacent turns and/or bands 4602 of the wire 4601 so that the device 4600 can be compressed onto and/or into a delivery system, and low enough that the resulting surface area contact over the length of the device 4600 does not provoke an adverse tissue response. However, it can also be beneficial to have a turn density that is sufficiently high (e.g., adjacent turns are longitudinally closer together) to prevent sagging and/or invagination of the airway wall between adjacent turns (especially during expiratory flow (e.g., exhalation) when the pressure around the outside of the airway are higher than the pressures within the airway), and to ensure sufficient surface area contact for reducing and/or avoiding relative motion and/or migration. As such, the turn density of the present technology can be optimized for delivery system loadability, minimal invagination of the airway wall between turns, minimal relative motion, and minimal local inflammatory response. In some embodiments, the device 4600 has a turn density of about 1 to about 4 turns per inch. In some embodiments, the device 4600 has a turn density of about 1.2 to about 3.5 turns per inch. In particular embodiments, the device 4600 has a turn density of about 1.8 to about 3 turns per inch. In FIG. 5, the device 4600 has a turn density of 3. FIG. 9 shows a device 5100 having a lower turn density of 1.8.

The expanded cross-sectional dimension of the device 4600 may be generally constant or vary along the length of the device 4600 and/or from loop to loop. For example, as discussed herein, the device 4600 can have varying cross-sectional dimensions along its length to accommodate different portions of the airway. For example, in some embodiments the device 4600 can have a diameter that decreases in a distal direction, thereby better approximating the natural distal narrowing of an airway lumen. The diameter may increase in a distal direction gradually over the length of the device 4600, or the device 4600 may have discrete portions with different diameters. For instance, the device 4600 can have a first portion and a second portion along its length. The first portion can have a first cross-sectional dimension that is configured to be positioned in a more distal portion of the airway (such as, for example, in a left or right main bronchus). The second portion can have a second cross-sectional dimension greater than the first cross-sectional dimension and configured to be positioned more proximally (such as in the trachea).

In some embodiments, the device 4600 can have a diameter that increases in a distal direction. The diameter may decrease gradually in a proximal direction over the length of the device 4600, or the device 4600 may have discrete portions with different diameters. For instance, the device 4600 can have a generally uniform diameter much of its length, then a larger diameter over the last distal 1-3 turns (which could be bands 4602 and/or a distal structure 4610). In some embodiments, the device 4600 has a first portion and a second portion along its length. The first portion can have a first cross-sectional dimension that is configured to be positioned in a more distal portion of the airway (such as, for example, in a left or right main bronchus). The second portion can have a second cross-sectional dimension less than the first cross-sectional dimension and configured to be positioned more proximally (such as in a trachea).

In some embodiments, the wire 4601 has a circular cross-sectional shape. In other embodiments, the wire 4601 may have other suitable cross-sectional shapes along its length (e.g., oval, rectangle, square, triangular, polygonal, irregular, etc.). In some embodiments, the cross-sectional shape of the wire 4601 varies along its length. Varying the cross-sectional shape of the wire 4601 may be beneficial to varying the mechanical performance of the device 4600 along its length (e.g., transition from lower to higher radial strength proximal to distal or vice versa). Alternatively or additionally, different cross-sectional shapes allows for different distributions of contact force on the airway wall. For example, a wire having an ovular cross-sectional shape will have greater contact area, wider distribution of contact force and, accordingly, lower contact stress at any point on the device 4600 as compared to a circular cross-section. Without being bound by theory, it is believed that is may be beneficial to utilize a cross-sectional shape with rounded edges, as rounded edges may present a less traumatic surface to the airway wall than straight edges. For example, while a wire having a rectangular cross-sectional shape and linear corners can be used with the present technology, in some cases it may be advantageous to utilize a rectangular wire with curved corners.

The wire 4601 can have a generally constant cross-sectional area along its length, or may have a varying cross-sectional area along its length. It may be beneficial to vary the cross-sectional area of the wire 4601, for example, to vary the radial force and/or flexibility of the device 4600 along its length. For instance, the device 4600 will have a lower radial force and/or be more flexible along portions in which the wire 4601 has a smaller cross-sectional area than along portions in which the wire 4601 has a greater cross-sectional area. In some embodiments, the wire 4601 has a diameter of no more than 0.005 inches, no more than 0.006 inches, no more than 0.007 inches, no more than 0.008 inches, no more than 0.009 inches, no more than 0.01 inches, no more than 0.011 inches, no more than 0.012 inches, no more than 0.013 inches, no more than 0.014 inches, and no more than 0.015 inches.

In some embodiments, the expanded cross-sectional dimension of the device 4600 in an unconstrained, expanded state (i.e., removed from the constraints of a delivery shaft, airway and sitting at rest on a table), can be oversized relative to the diameter of the native airway lumen. For example, the expanded, unconstrained cross-sectional dimension of the device 4600 can be at least 1.5× the original (non-collapsed) diameter of the airway lumen in which it is intended to be positioned. In some embodiments, the device 4600 has an expanded, cross-sectional dimension that is about 1.5× to 6×, 2× to 5×, or 2× to 3× the diameter of the original airway lumen. Without being bound by theory, it would be clinically beneficial to expand the airway lumen to the greatest diameter possible. A large airway diameter will allow for more efficient release of trapped air, thereby optimizing improvement in pulmonary function (for example, as measured by outflow, FEV, and others).

Given that the cartilaginous support in bronchial airways tends to decline proximal to distal, it may be beneficial to have a device with variable turn density, wherein the turn density in the distalmost portion of the device is greater than the turn density in the proximalmost portion of the device. This device configuration, with greater turn density distally and lower turn density proximally, may optionally include lower radial stiffness distally and greater radial stiffness proximally.

The distal structure 4610 is the first portion of the device 4600 to be deployed in the airway lumen. As a result, the distal structure 4610 can be similar to the bands 4602, but adapted to provide greater circumferential force and a soft, atraumatic landing structure. The final apex 4616 of the wire 4601, for example, can be angled so as to orient the distal terminus 4620 of the wire 4601 proximally, and have a greater radius of curvature in its relaxed, unconstrained state than the other apices so as to provide a rounder, softer bend for first contacting the airway wall. In some embodiments, the distal apex 4616 has approximately the same radius of curvature in the relaxed, unconstrained state as the rest of the apices. Additionally or alternatively, the distal terminus 4620 of the wire 4601 can comprise other atraumatic elements, such as a ball (having a cross-sectional dimension only slightly greater than a cross-sectional dimension of the wire 4601) and/or a looped portion of the wire 4601. To enable a greater anchoring force at the distal end portion 4600b of the device 4600, the third valley 4606c of the distal structure 4610 can have a greater radius of curvature so as to substantially align the final apex 4616 (which is a peak) with the second-to-last peak 4604b of the distal structure 4610.

The proximal end portion 4600a of the device 4600 can comprise a single, proximally-extending strut 4624 and a free proximal terminus 4622. Similar to the distal terminus 4620, the proximal terminus 4622 can extend in a proximal direction to limit trauma to the airway wall. The free proximal terminus can also be beneficial for retrieval of the device 4600, if necessary.

The wire 4601 can be any elongated element, such as a wire (e.g., having a circular or ovular cross-sectional shape), a coil, a tube, a filament, a single interwoven elongated element, a plurality of braided and/or twisted elongated elements, a ribbon (have a square or rectangular cross-sectional shape), and/or others. As such, the term “wire,” as used herein, refers to the traditional definition of a wire (e.g., metal drawn out into the form of a thin flexible thread or rod), as well as the other elongated elements detailed herein. The wire 4601 can be cut from a sheet of material then wound around a mandrel into the three-dimensional configuration. In some embodiments, the device 4600 is formed by cutting a tube such that the only remaining portions of the tubular sidewall comprise the wire 4601. The sheet and/or tube can be cut via laser cutting, electrical discharge machining (EDM), chemical etching, water jet, air jet, etc. The wire 4601 can also comprise a thin film formed via a deposition process. The elongated member 102 can be formed using materials such as nitinol, stainless steel, cobalt-chromium alloys (e.g., 35N LT®, MP35N (Fort Wayne Metals, Fort Wayne, Indiana)), Elgiloy, magnesium alloys, tungsten, tantalum, platinum, rhodium, palladium, gold, silver, or combinations thereof, or one or more polymers, or combinations of polymers and metals. In some embodiments, the wire 4601 may include one or more drawn-filled tube (“DFT”) wires comprising an inner material surrounded by a different outer material. The inner material, for example, may be radiopaque material, and the outer material may be a superelastic material.

The cross-sectional area of the wire 4601 can be selected based on several factors, such as turn density, radial force, and ability to radially compress for delivery. All else equal (such as turn density, length of wire, wire material, etc.), the greater the cross-sectional area of the wire 4601, the greater the radial force exerted on the airway wall. However, the greater the cross-sectional area of the wire 4601 and associated radial force, the more difficult it is to compress the device 4600 into and/or onto a delivery system. As such, the wire 4601 of the present technology has a cross-sectional area that, along with the turn density of the wire 4601, provides the device 4600 with a radial force sufficient to maintain airway patency, resist strain and associated cycle fatigue from anatomical loading during respiration and coughing and reduce and/or eliminate relative motion while still allowing the device 4600 to be compressed down to a diameter of less than 3 mm, and in some cases less than 2 mm.

It can be advantageous to have a radial force high enough to resist migration and, via improved wall apposition, reduce relative motion between the device 4600 and the airway wall, as relative motion can irritate the wall tissue and cause a foreign body response that may contribute to occlusion of the airway. The radial force must also be sufficient to maintain patency of the airway, and in some cases dilate the airway to a diameter that is larger than the native diameter of the airway, for example this could be 2-3 times greater. The radial force exerted by the device 4600 on the airway wall is determined, at least in part, by the turn density of the device 4600 and the cross-sectional area of the wire 4601. For example, the greater the cross-sectional area of the wire 4601, the greater the radial force. The greater the turn density of the device 4600, the greater the radial force. Likewise, the lower the cross-sectional area of the wire 4601, the lower the radial force. The lower the turn density of the device 4600, the lower the radial force. The devices 4600 of the present technology can have a radial force per unit length of no more than 7 g/mm, no more than 6 g/mm, no more than 5 g/mm, no more than 4 g/mm, no more than 3 g/mm, no more than 2 g/mm, or no more than 1 g/mm. In some embodiments, the device 4600 has a radial force per unit length of from about 1 to about 5 g/mm. The radial force required to hold open a collapsed airway and maintain patency during respiration is less than that required by stents used to push or hold back tumor growth or atherosclerosis. Such conventional stents typically have a radial force per unit length of about 10 g/mm or greater.

The device 4600 may be configured to have minimal surface area contact with the airway wall to reduce the amount of foreign body response (such as inflammation and granulation tissue) and risk of airway occlusion. As used in this discussion, “contacting surface area” refers to the surface area of the portion of the device 4600 that contacts the inner surface of the airway wall, which is less than the total surface area of the wire 4601. Minimizing the contacting surface area of the device 4600 can also be beneficial for limiting and/or avoiding occlusion of other distal branch openings, and for enabling more efficient mucociliary clearance. The contacting surface area of the device 4600, however, also impacts the device's ability to resist migration and relative motion. As such, the devices 4600 of the present technology can be configured to have a contacting surface area that is low enough to minimize (or localize) an adverse tissue reaction and allow for sufficient mucociliary clearance, but high enough to provide good contact with the airway and resist motion. The devices 4600 of the present technology can have, for example, a contacting surface area of no more than 20%, no more than 19%, no more than 18%, no more than 17%, no more than 16%, no more than 15%, no more than 14%, no more than 13%, no more than 12%, no more than 11%, no more than 10%, no more than 9%, no more than 8%, no more than 7%, no more than 6%, or no more than 5%. Said another way, the porosity of the device 4600 can be at least 80%, at least 81%, at least 82%, at least 83%, at least 84%, least 85%, at least 86%, at least 87%, at least 88%, at least 89%, at least 90%, at least 91%, at least 92%, at least 93%, at least 94%, or at least 95%.

In some embodiments, regardless of whether the wire 4601 is made of and/or includes a radiopaque material, the device 4600 can include one or more radiopaque markers. The radiopaque markers, for example, can be disposed at one or both ends of the device 4600 to facilitate accurate positioning and placement.

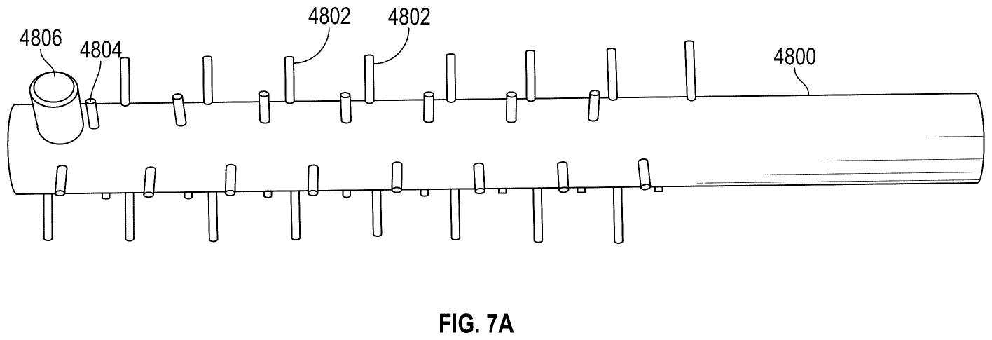

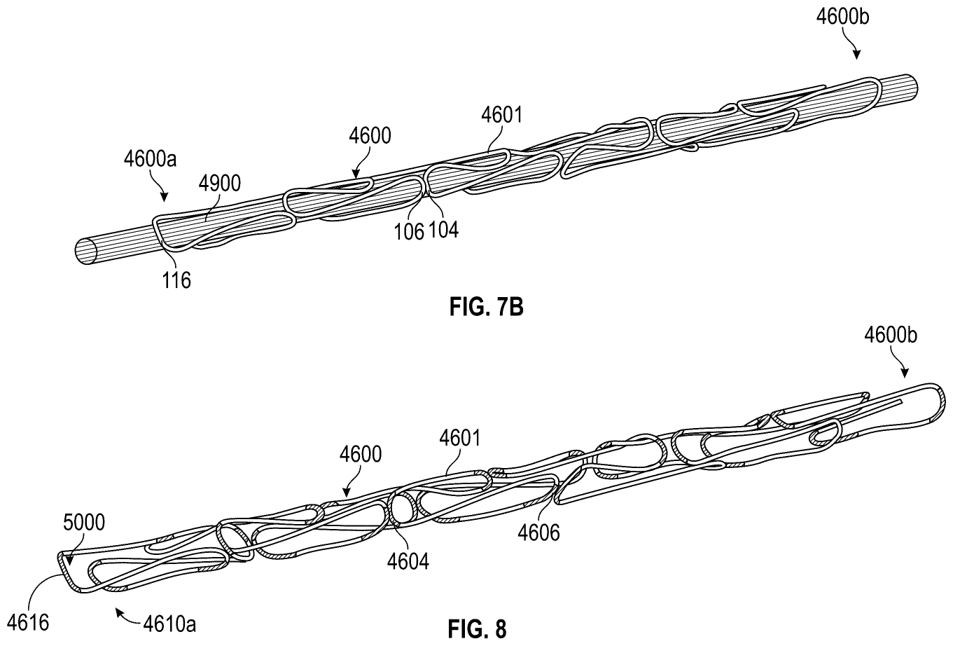

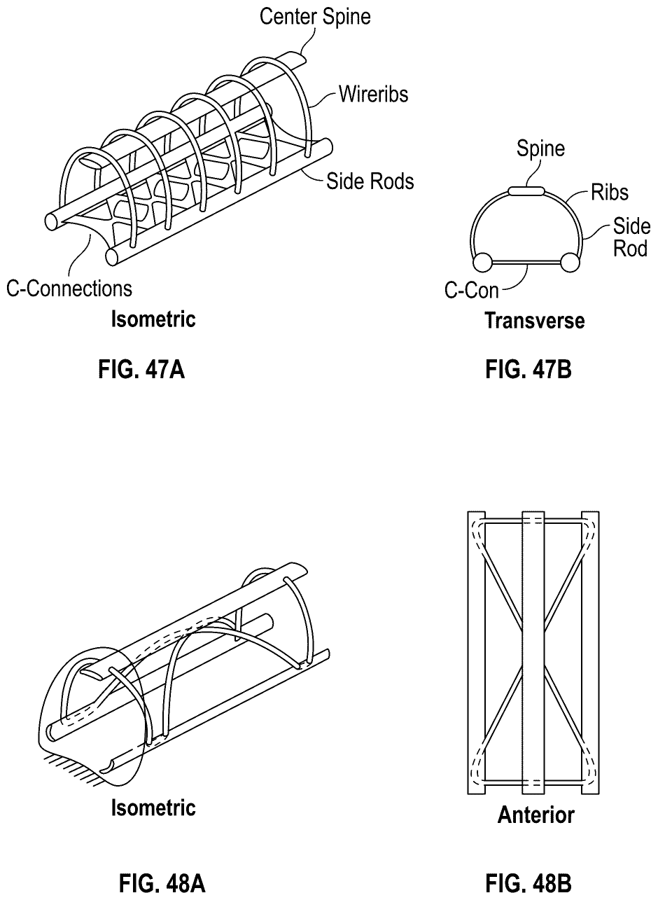

In some embodiments, the device 4600 is manufactured by wrapping the wire 4601 around a mandrel according to a predetermined wrap pattern, then heat setting the wire 4601 while held in place on the mandrel so that when the wire 4601 is removed from the mandrel, the wire 4601 substantially maintains its on-mandrel shape. FIG. 7 shows a mandrel 4800 configured for use in manufacturing the devices of the present technology. As shown in FIG. 7, the mandrel 4800 can be generally cylindrical and include a plurality of posts 4802 extending radially away from an outer surface of the mandrel 4800. The posts 4802 can be arranged in a pattern that produces a desired wrap geometry. The radius of curvature of the posts 4802, for example, can determine the radius of curvature of the apices. FIG. 47 shows a portion of the wire 4601 wrapped around one of the posts 302. Different apices along the device 4600 can have the same radii of curvature or different radii of curvature.

In some cases it may be beneficial to use posts having a radius of curvature that closely resembles a shape of the apices when the device 4600 is compressed down onto and/or into a delivery system. FIG. 7 shows the device 4600 in a radially compressed state, positioned over an elongated delivery member 4900. As the device 4600 gets radially compressed, the two struts 4608 adjacent any given peak 4604 or valley 4606 get pinched together, thereby placing a strain on the attached apex. FIG. 8, for example, shows a finite element analysis performed on the device 4600 to calculate cyclic strains, since the device 4600, when implanted, will exhibit cyclic strain in the form of respiration, coughing, and others. As shown in FIG. 8, the strain amplitude peaked at the distal portion where the apex 4616 was heat set to have a radius of curvature that was greater than that of the other apices (such as peak 4604 and valley 4606). The apices that were heat set around smaller diameter posts (having small radii of curvature) were projected to experience less strain and fatigue compared to the distal apex 4616 when forced into a compressed state. Accordingly, it may be desirable for the apices to have an average radius of curvature that is no greater than 2.5 mm (e.g., 2.5 mm or less, 2 mm or less, 1 mm or less, 0.5 mm or less, or within a range from 0.35 mm to 0.60 mm).

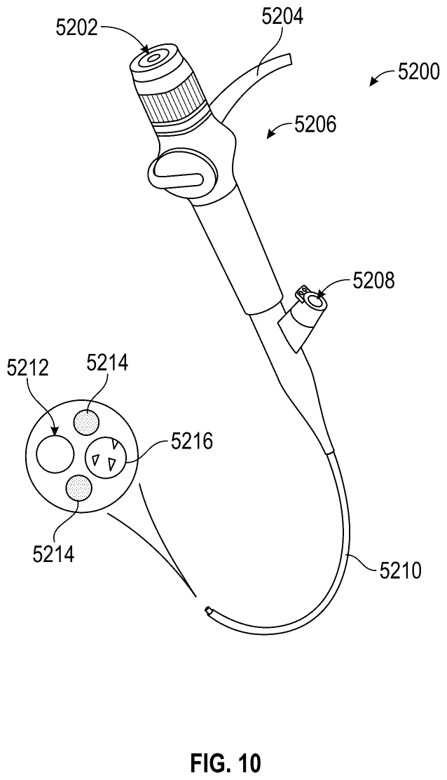

The device 4600 can be configured for delivery through a working channel of a bronchoscope. An example bronchoscope 5200 is shown in FIG. 10. As shown, the bronchoscope 5200 can have a handle with an eyepiece or camera head 5202, a cable 5204 for the light source used for image processing, a suction portion 5206, and a working channel port 5208. The bronchoscope includes an elongated shaft 5210 configured to be advanced through a patient's nose and down through their trachea to the lungs. The shaft 5210 includes several lumens, including a lumen 5216 supporting a camera or fiber optic cable bundle, lumens 5214 supporting the light source, and the outlet of the working channel 5212. The working channel lumen can have a diameter of about 3 mm or less.

As shown in FIG. 11, the elongated shaft 5210 of the bronchoscope 5200 (FIG. 10) can be advanced through the trachea T until reaching the distal end portion of a desired treatment site (e.g., the distal end of the weakened and/or narrowed portion). The device 4600 can then be deployed in a distal to proximal direction. FIG. 12 shows the device 4600 after deployment. In the embodiment of FIG. 12, the distal end portion of the device 4600 is positioned in a distal portion of the trachea T with the proximal end portion of the device 4600 positioned in a proximal portion of the trachea. Depending on the pathophysiology presented by the patient, the device 4600 may be sized (e.g., length, cross-sectional shape, cross-sectional dimension, etc.) for placement in the trachea such that the distal end portion of the device 4600 is positioned in an intermediate portion of the trachea T with the proximal end portion of the device 4600 positioned in an intermediate or proximal portion of the trachea. Additionally or alternatively, the device 4600 may be sized for placement in the airways such that the distal end portion of the device 4600 is positioned in the left or right main bronchus and the proximal end portion of the device in the trachea. In some embodiments, the device 4600 may be sized for placement in the airways such that both the proximal and distal end portions of the device 4600 are positioned in the left main bronchus or the right main bronchus. In yet further embodiments, the device 4600 may be sized for placement in the airways such that one end of the device 4600 is positioned in the left main bronchus and the other end of the device is positioned in the right main bronchus. The device 4600 and/or wire 4601 (FIG. 5) can be configured to self-expand to a preset configuration and/or diameter. In some embodiments, the wire 4601 is not heat set and/or configured to self-expand. For example, in some embodiments, the device 4600 and/or wire 4601 is balloon expandable. In some embodiments, the device 4600 and/or wire 4601 is balloon expandable and self-expanding.

In some embodiments, the device 4600 can be deployed to a discrete length (e.g., 20, 30, 40, 50, 60 cm, etc.) or, given the axial flexibility of the device 4600, the device 4600 and/or delivery system can be designed for variable length deployment (e.g., each device can be designed to be deployed to up to +/−5 cm of its nominal length) to accommodate variability in patient anatomy. According to some embodiments, the present technology includes multiple devices 4600 delivered in series. The devices placed in series may have different lengths to accommodate and fit different treatment lengths. The multiple devices can overlap, touch, or be spaced apart. If spaced apart, the devices may be spaced no more than a predetermined distance apart in the airway (e.g., 5 mm, 1.0 cm, 1.5 cm, 2.0 cm).

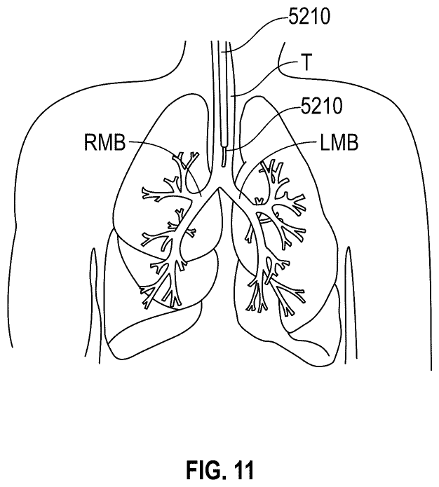

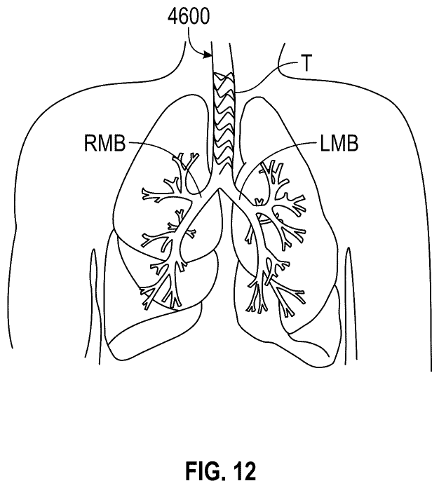

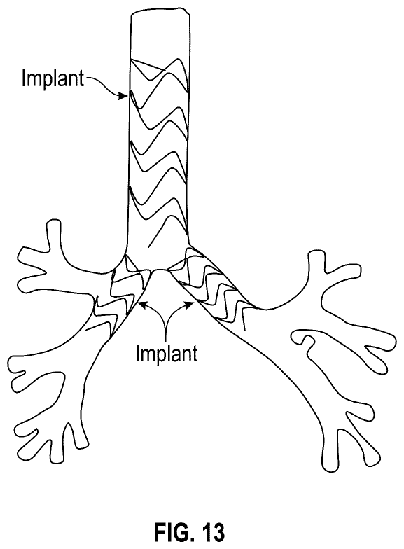

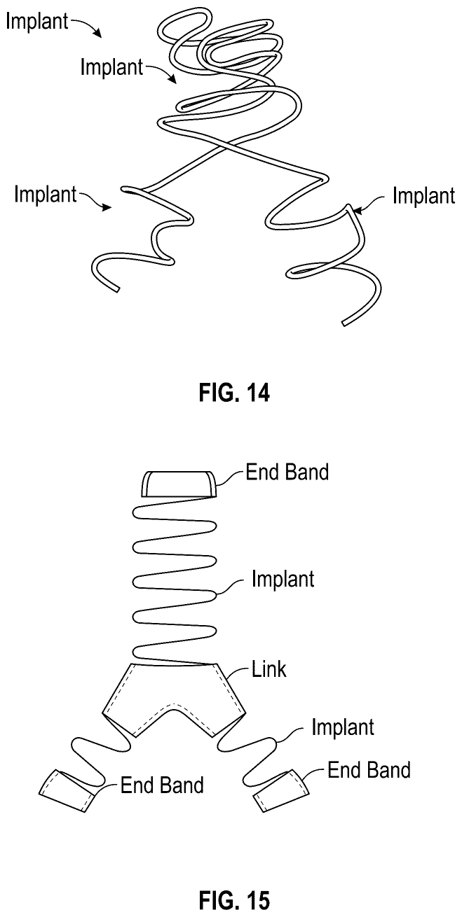

The deployment process described above can be repeated with additional implants at different respective airway regions. For example, in some embodiments, a first implant (e.g., any of the implants disclosed herein) can be deployed within the trachea, and a second implant can be deployed in the left or right main bronchus. Certain methods may require a third implant for deployment in the other of the left or right main bronchus. FIG. 13, for example, shows three separate implants, each deployed in one of the trachea, left main bronchus, and right main bronchus. The first, second, and third implants can have the same or different lengths, same or different cross-sectional dimensions, same or different cross-sectional shapes, same or different COF, same or different RRF, and same or different rigidity/flexibility.

In some embodiments, the ends of the first, second, and/or third implants are spaced apart (shown shown). In other embodiments, the first, second, and/or third implants may overlap along their lengths (not shown). For example, the tracheal implant and bronchial implant can slightly overlap at their ends. It may be beneficial to minimize the amount of overlap so as to avoid a higher density of material (and thus greater surface area contact with the airway wall) at a single location.

According to some embodiments, for example as shown in FIG. 13, the implant can be a single piece that has branches for the trachea and both left and right main bronchi. This configuration may advantageously prevent a choke point from moving distally once trachea is supported. The single piece may be formed by a single wire, or by multiple wires that are joined together.

For those embodiments including two or more implants, the two or more implants may be held together by a link. Like the implants, the link can have a collapsed and/or compressed state for delivery through a sheath to the treatment site and an expanded state for implantation in the airway. In some embodiments, the link is reinforced with an embedded superelastic wire to reduce and/or prevent collapse at the junction between the connected implants. The embedded wire may be configured to provide elastic resilience such that the link is biased towards its expanded state and/or will resist airway collapse. In certain embodiments, the link may be configured to conform to the carina. According to several embodiments, the link is impregnated with a therapeutic agent. An inner surface of the link (i.e., facing the airway lumen) may have a lubricious coating, material, or other treatment configured to facilitate mucous removal. An outer surface of the link (e.g., configured to face and contact the native airway tissue) may have a coating, material, or other treatment that enhances hydrophilic properties to resist migration. The link may provide several benefits such as, for example, protecting local tissue from the wire ends of the implants, reducing and/or preventing migration, and providing strain relief to reduce tissue trauma.

The link may be configured to join two implants (trachea to bronchus or bronchus to bronchus) or may be configured to join three implants. FIG. 15, for example, shows an implanted device that includes multiple implants (one in the trachea and one in each of the left and right main bronchus) joined by a Y-shaped link. The Y-shaped link may define a Y-shaped lumen and be formed of silicone or other material in which the ends of the implants are embedded. For radially asymmetric devices (such as C-shaped or D-shaped implants (discussed below)), the Y-shaped link may help maintain radial orientation.

In certain embodiments, the implant ends can be inserted into the Y-shaped link in situ. In such embodiments, the sidewall defining the Y-shaped link may include a groove and/or channel configured to receive the wire of the implant. The groove and/or channel may be in one or both of the two distal openings and one proximal opening. Additionally or alternatively, the Y-shaped link may already be joined to two of the three implants and the remaining free end may include a groove and/or channel for the remaining implants. In some embodiments the groove and/or channel is threaded, and in other embodiments the groove and/or channel is configured to mate with the wire of the implant via a friction and/or form fit.

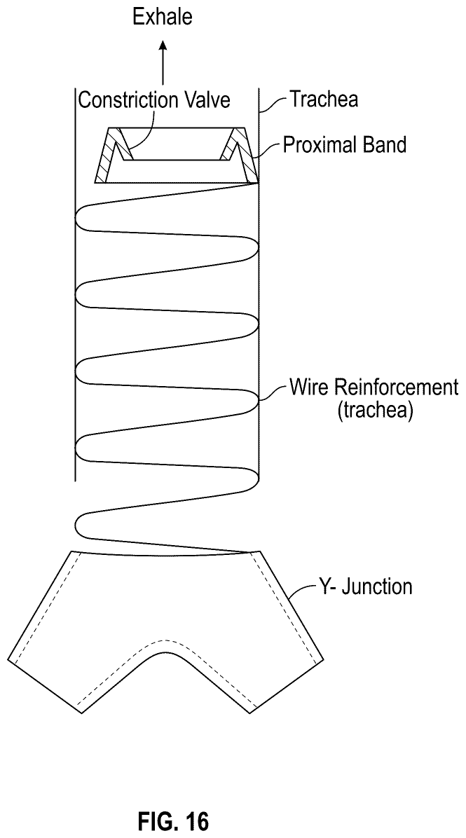

Any of the implants disclosed herein can include one or more bands, for example as shown in FIG. 15. The band may be positioned at one or both ends of the implant (as shown) and/or at an intermediate portion. The band can include any of the above-described properties of the Y-shaped link (other than the shape). In some embodiments, the band can include a one-way constriction valve that fully opens during inhalation and partially closes during exhalation. The band can be placed, for example, at a proximal end portion of the trachea to beneficially provide air resistance to slightly increase air pressure in the trachea distal to the proximal band, which in turn may decrease a pressure gradient that causes tracheal or bronchial collapse. Increasing air resistance with a constriction valve may have a similar effect to pursed lip breathing. The constriction valve may have a flap that is hinged on a proximal end of the valve so that the valve catches air during exhalation, causing a decrease in the airway lumen diameter such that the airway fully opens during inhalation. The constriction valve may cover a portion or all of the circumference of the band. The lumen through the constriction valve may be reduced in a range of 5 to 20% when in its closed position compared to its open position (see FIG. 16).

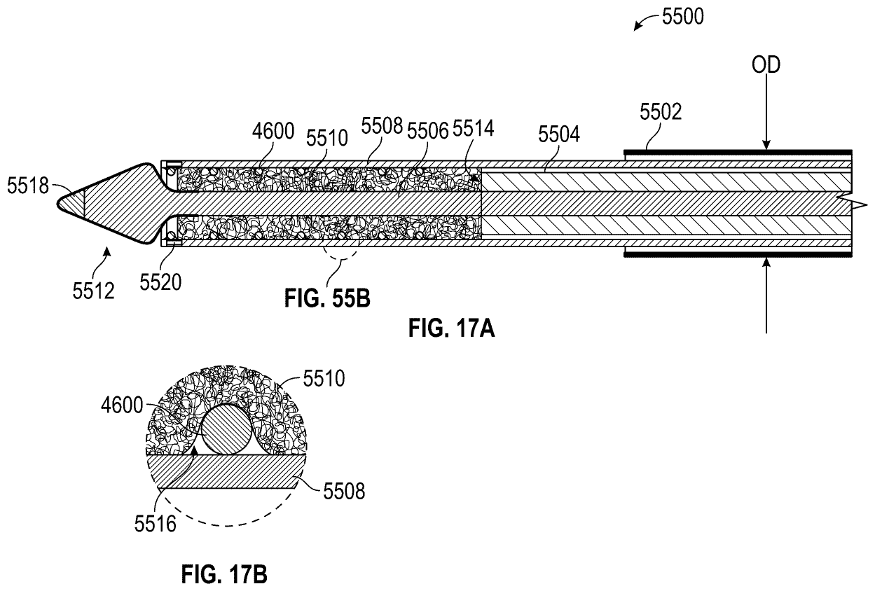

FIG. 17A shows the distal portion of a delivery system 5500 configured in accordance with several embodiments of the present technology. The delivery system 5500 can be configured for delivery through a working channel of a bronchoscope. One or more components of the delivery system 5500 and/or the bronchoscope can be coupled to a robotic system (including any of those disclosed herein, and others) which controls the movement of the respective one or more components of the delivery system 5500 and/or bronchoscope). In some embodiments the delivery system 5500 has an outer diameter of no greater than 3 mm. In some embodiments, the delivery system 5500 has an outer diameter of no greater than 2 mm.

The system 5500 can include an outer sheath 5502, an inner sheath 5508 configured to be slidably disposed within the outer sheath 5502, and an elongated shaft or other delivery member 5506 disposed within the inner sheath 5508. One, some, or all of the outer sheath 5502, the inner sheath 5508, and the elongate shaft 5506 can be coupled to an instrument driver of a robotic system. As such, rotation, translation, or other movement of one, some, or all of the outer sheath 5502, the inner sheath 5508, and the elongate shaft 5506 can be controlled by the instrument driver and/or robotic system. The outer sheath 5502 can be configured to encase the entire delivery system and engage with the working channel 5212 of the bronchoscope 5200. For example, in some embodiments a proximal end of the outer sheath 5502 is fixed to a handle (not shown) of the delivery system 5500. The inner sheath 5508 is configured to be retracted to expose and deploy the device 4600. In at least some embodiments, the axial position of the delivery member 5506 is fixed relative to the axial position of the outer sheath 5502. For example, a proximal end of the delivery member 5506 can be fixed to the handle of the delivery system 5500. Moreover, the overall delivery system 5500 with the exception of the inner sheath 5508 can be fixed to the bronchoscope 5200. In other embodiments, counterpart delivery systems can have other suitable combinations of movable and fixed components.

In some embodiments, the system 5500 optionally includes a tapered, atraumatic tip 5512 at the distal end of the elongated member 5506. The system 5500 can further include a proximal stop 5504 positioned around the elongated member 5506 and within the inner sheath 5508. The proximal stop 5504 can have a distal-facing surface 5514 configured to abut a proximal end of the device 4600. In some embodiments, the system 5500 optionally includes a pad or other conformable member 5510 radially positioned between the device 4600 and the elongated member 5506. The conformable member 5510 can be more resilient than the elongated member 5506. The conformable member 5510 can have an intimate engagement with the device 4600 when it is radially compressed. For example, as shown in FIG. 17B, the conformable member 5510 can form an indentation 5516 around the device 4600 that helps the device 4600 maintain its axial position. In this or another manner, the device 4600 can be ‘tacked’ into the conformable member 5510 to hold it in place until the inner sheath 5508 is fully retracted.

In at least some cases, the delivery system 5500 includes features to facilitate fluoroscopic and/or bronchoscopic visualization during delivery and/or deployment of the implant 4600. For example, the delivery system 5500 can include a first radiopaque marker 5518 at a distalmost portion of the tip 5512 to indicate a distalmost feature of the delivery system 5500. The first radiopaque marker 5518, for example, can be a cap or an embedded plug. The delivery system 5500 can further include a second radiopaque marker 5520 at a distalmost portion of the inner sheath 5508 to facilitate estimating a location of a distal end of the device 4600 during delivery and deployment. The second radiopaque marker 5520, for example, can be an annular band. In addition or alternatively, the delivery system 5500 can include pad printed lines or other visual features (not shown) at an outer surface of the inner sheath 5508. These features can facilitate bronchoscopic visualization. For example, one line can be at the proximal end of the device 4600 to indicate where relative to an airway region the proximal end of the device 4600 will be placed after deployment. Furthermore, different indicators can be used to indicate proximal ends of devices of different lengths. For example, one circumferential line can indicate the proximal end of a 70 mm device, two circumferential lines can indicate the proximal end of a 85 mm device, three circumferential line can indicate the proximal end of a 100 mm device, etc.

The elongated shaft 5210 of the bronchoscope 5200 can be advanced through the trachea and bronchial tree (e.g., manually or via robotic assistance) to the treatment site. The position at which the elongated shaft 5210 ceases advancement may be different depending on the bronchoscope being used. For a typical bronchoscope with a 5-6 mm diameter, this would occur in most patients in the 3rd to 6th generation bronchi. The delivery system 5500 can then be advanced distally (e.g., manually or via robotic assistance) through the distal opening of the working channel 5212 such that the outer sheath 5502 is exposed within the airway lumen. The delivery system 5500 can be advanced distally until the distal end portion of the outer sheath 5502 is positioned within a portion of the airway at a distal end portion of the weakened and/or mis-shaped region The distal end portion of the outer sheath 5502, for example, may be positioned within the trachea, the left main bronchus, or the right main bronchus. With the outer sheath 5502 and elongated delivery member 5512 held in position, the inner sheath 5508 can be retracted (e.g., manually or via robotic assistance) to expose and deploy the device 4600 at a desired location.

In some embodiments, only the bronchoscope is coupled to and under the control of the robotic system and the delivery system remains under manual control. In some embodiments, only the delivery system is coupled to and under the control of the robotic system and the bronchoscope remains under manual control. In several embodiments, both the bronchoscope and the delivery system are coupled to and under the control of the robotic system. In those embodiments in which the delivery system is under robotic control, one, some, or all of the movable components of the delivery system can be coupled to and under robotic control. The moveable components not under robotic control (if any) can be manually manipulated. The foregoing options apply to the delivery system disclosed with respect to FIGS. 55A-55B as well as any other delivery system disclosed herein.

It will be appreciated that other delivery systems are within the scope of the present technology. For example, the implants of the present technology can be deployed by any of the delivery systems disclosed in PCT Application No. TBD [Attorney Docket No. APH. 007WO], titled, filed concurrently herewith, and PCT Application No. TBD [Attorney Docket No. APH. 005WO], titled METHODS AND SYSTEMS FOR TREATING PULMONARY DISEASE, filed concurrently herewith, both of which are incorporated by reference herein in their entireties. Moreover, the bronchoscope 5200 and delivery system 5500 can be used with any of the expandable devices disclosed herein.

Additional examples of expandable devices, systems, and methods for treating ECAC and/or devices, systems, and methods for modifying an airway wall can be found, for example, in U.S. Pat. No. 9,592,138, filed Sep. 13, 2015, titled PULMONARY AIRFLOW, PCT Application No. PCT/US22/73962, titled ENDOBRONCHIAL IMPLANTS AND RELATED TECHNOLOGIES, filed Jul. 20, 2022, which is incorporated by reference herein in its entirety.

### III. ADDITIONAL EXAMPLES

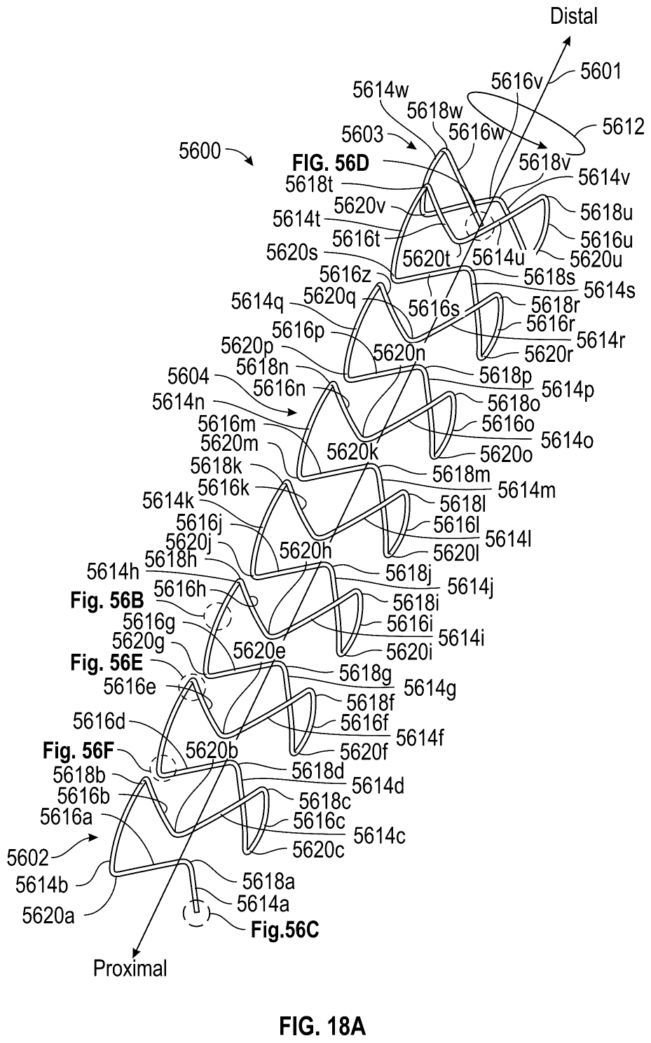

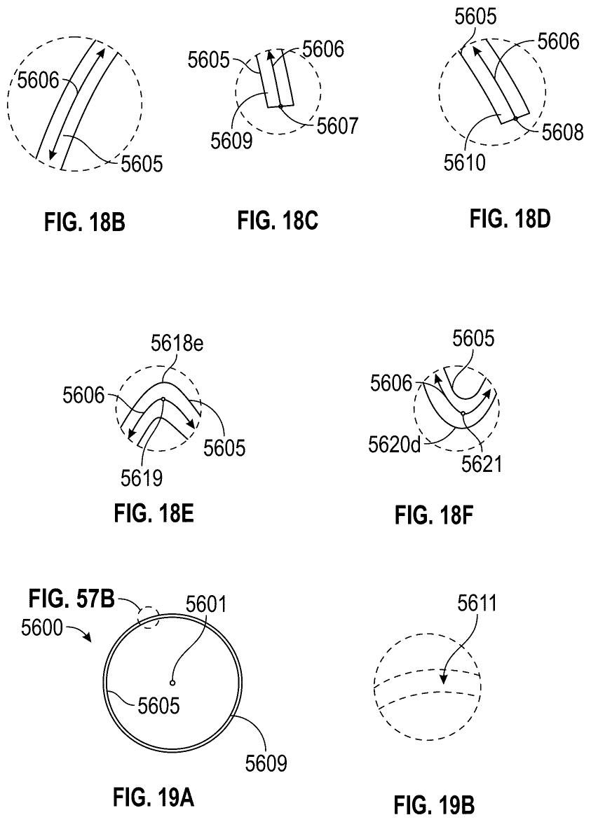

FIGS. 18A, 19A and 20 are a perspective view, an end view, and a profile view, respectively of the implant 5600 in accordance with at least some embodiments of the present technology. FIGS. 18B-18F are callouts corresponding to FIG. 18A. FIG. 19B is a callout corresponding to FIG. 19A. In FIGS. 18A-20, the implant 5600 is in an unconstrained state. This can be a state the implant 5600 assumes in the absence of external sources of constraint, such as a sheath during delivery of the implant 5600 or a wall of a bronchial tree after deployment of the implant 5600. Features of the implant 5600 are described herein with respect to the implant 5600 in this unconstrained state unless otherwise specified. With reference to FIGS. 18A-20 together, the implant 5600 can be elongate with a longitudinal axis 5601. The implant 5600 can include a proximal end portion 5602 and a distal end portion 5603 spaced apart from one another along the longitudinal axis 5601. Between the proximal end portion 5602 and the distal end portion 5603 along the longitudinal axis 5601, the implant 5600 can include an intermediate portion 5604. The overall implant 5600 can be configured to configured to be deployed at a treatment location within a bronchial tree of a human subject. Aspects of examples of this deployment are described in detail below. In at least some cases, the proximal end portion 5602 and the distal end portion 5603 are configured to be deployed at different respective airways. For example, the proximal end portion 5602 can be configured to be deployed at a first airway and the distal end portion 5603 can be configured to be deployed at a second airway of a generation greater than a generation of the first airway. The respective generations of the first and second airways can be different by 1, 2, 3, 4, 5, 6, or an even greater number depending on features such as the length and diameter of the implant 5600. The first airway can be of a generation 2 or greater, such as 2, 3, 4, 5 or 6.

The implant 5600 can further include a wire 5605 extending along a wire path 5606. The wire path 5606 can extend between a first end 5607 at the proximal end portion 5602 and an opposite second end 5608 at the distal end portion 5603. The wire path 5606 can be continuous between the first end 5607 and the second end 5608. Furthermore, the wire 5605 can include a first terminus 5609 at the first end 5607 and a second terminus 5610 at the second end 5608. The wire path 5606 can extend in a circumferential direction 5612 about the longitudinal axis 5601. Some, most, or all of the wire 5605 and the wire path 5606 can be within a tubular region 5611 coaxially aligned with the longitudinal axis 5601. In the illustrated embodiment, the tubular region 5611 has a circular cross-sectional shape perpendicular to the longitudinal axis 5601. In other embodiments, a counterpart of the tubular region 5611 can be ovoid, triangular with rounded corners, square with rounded corners, otherwise polygonal with rounded corners, or have another suitable shape perpendicular to a counterpart of the longitudinal axis 5601. Furthermore, although the longitudinal axis 5601 and the tubular region 5611 are straight in the illustrated embodiment, in other embodiments, the longitudinal axis 5601 and the tubular region 5611 can be curved. For example, a counterpart of the implant 5600 can be curved, angled, serpentine, or have another suitable nonlinear shape. Such a nonlinear shape, for example, can be selected to correspond to a shape of an airway region in which the counterpart of the implant 5600 is to be deployed.

With reference again to FIGS. 18A-20, in the illustrated embodiment the overall wire path 5606 between the first end 5607 and the second end 5608 include includes seven complete turns about the longitudinal axis 5601. In other embodiments, a counterpart of the wire path 5606 can include another suitable number of turns, such as another suitable number of turns corresponding to a desired pitch and overall length of a counterpart of the implant 5600. In at least some embodiments, the wire path 5606 at the intermediate portion 5604 includes three or more complete turns, such as four turns, five turns, six turns, or more. In these and other embodiments, the wire path 5606 at the proximal end portion 5602 can include one complete turn closest to the first end 5607. Similarly, the wire path 5606 at the distal end portion 5603 can include one complete turn closest to the second end 5608. Delineation between the proximal end portion 5602, the distal end portion 5603, and the intermediate portion 5604 can be based on turns and/or based on segments of the longitudinal axis 5601. For example, the proximal end portion 5602 can be coextensive with a proximalmost 10% of the longitudinal axis 5601, the distal end portion 5603 can be coextensive with a distalmost 10% of the longitudinal axis 5601, and the intermediate portion can be coextensive with an intermediate 80% longitudinal axis 5601. Alternatively, the proximal end portion 5602 can be coextensive with a proximalmost 15% of the longitudinal axis 5601, the distal end portion 5603 can be coextensive with a distalmost 15% of the longitudinal axis 5601, and the intermediate portion can be coextensive with an intermediate 70% longitudinal axis 5601. Other suitable delineations are also possible.

The wire 5605 can include first legs 5614 (individually identified as first legs 5614a-5614w) and second legs 5616 (individually identified as second legs 5616a-5616w) alternatingly disposed along the wire path 5606. The first legs 5614a-5614w can extend distally in the circumferential direction 5612 while the second legs 5616a-5616w extend proximally in the circumferential direction 5612. In the illustrated embodiment, all of the first legs 5614a-5614w and all of the second legs 5616a-5616w have these specified orientations. In other embodiments, a counterpart of the wire 5605 can include only some (e.g., most, all but one, all but two, etc.) counterparts of the first legs 5614a-5614w and/or counterparts of the second legs 5616a-5616w having the specified orientations. For example a counterpart of the wire 5605 can include counterparts of the first legs 5614a-5614w and counterparts of the second legs 5616a-5616w having the specified orientations only at a counterpart of the intermediate portion 5604, but not at a counterpart of the proximal end portion 5602 and/or not at a counterpart of the distal end portion 5603. Furthermore, in the illustrated embodiment and in at least some other embodiments, the first legs 5614a-5614w and the second legs 5616a-5616w and counterparts thereof can have any suitable features of corresponding portions of other devices described herein.

With reference again to FIGS. 18A-20, the wire 5605 can include first apex portions 5618 (individually identified as first apex portions 5618a-5618w) disposed at respective first apex points 5619 along the wire path 5606. The wire 5605 can also include second apex portions 5620 (individually identified as second apex portions 5620a-5620v) disposed at respective second apex points 5621 along the wire path 5606. In at least some cases, the first legs 5614a-5614w and the second legs 5616a-5616w are alternatingly disposed along the wire path 5606. Furthermore, the first legs 5614a-5614w and the second legs 5616a-5616w can be interspersed among the first apex portions 5618a-5618w and the second apex portions 5620a-5620v along the wire path 5606. As shown in FIG. 18A, the first apex portions 5618a-5618w can point distally (i.e., more toward the distal end portion 5603 than toward the proximal end portion 5602 along the longitudinal axis 5601). Correspondingly, portions of the wire 5605 nearest to the first apex portions 5618a-5618w can extend away from the first apex portions 5618a-5618w proximally. Similarly, the second apex portions 5620a-5620v can point proximally (i.e., more toward the proximal end portion 5602 than toward the distal end portion 5603 along the longitudinal axis 5601). Correspondingly, portions of the wire 5605 nearest to the second apex portions 5620a-5620v can extend away from the second apex portions 5620a-5620v distally. In the illustrated embodiment and in at least some other embodiments, the first apex portions 5618a-5618w and the second apex portions 5620a-5620v and counterparts thereof can have any suitable features of corresponding portions of other devices described herein.

The overall implant 5600, the proximal end portion 5602, the distal end portion 5603, and/or the intermediate portion 5604 can consist essentially of the wire 5605. Furthermore, the wire 5605 throughout the implant 5600, at the proximal end portion 5602, at the distal end portion 5603, and/or at the intermediate portion 5604 can consist essentially of various combinations of the first legs 5614a-5614w, the second legs 5616a-5616w, the first apex portions 5618a-5618w, and the second apex portions 5620a-5620v. In the illustrated embodiment, the proximal end portion 5602 includes the four of the first legs 5614 (first legs 5614a-5614d), three of the second legs 5616 (second legs 5616a-5616c), three of the first apex portions 5618 (the first apex portions 5618a-5618c), and three of the second apex portions 5620 (the second apex portions 5620a-5620c). These components correspond to a portion of the wire 5605 extending along a single complete turn of the wire path 5606 closest to the first end 5607 but with the first leg 5614d extending slightly beyond this turn along the wire path 5606 toward the second end 5608. In the illustrated embodiment, the distal end portion 5603 includes three of the first legs 5614 (first legs 5614u-5614w), three of the second legs 5616 (second legs 5616u-5616w), three of the first apex portions 5618 (the first apex portions 5618u-5618w), and two of the second apex portions 5620 (the second apex portions 5620u-5620v). These components correspond to a portion of the wire 5605 extending along a single complete turn of the wire path 5606 closest to the second end 5608 but with the second leg 5616u extending slightly beyond this turn along the wire path 5606 toward the first end 5607. Finally, in the illustrated embodiment, the intermediate portion 5604 includes 16 of the first legs 5614 (the first legs 5614c-5614t), 17 of the second legs 5616 (the second legs 5616d-5616t), 17 of the first apex portions 5618 (the first apex portions 5618d-5618t), and 17 of the second apex portions (the second apex portions 5620d-5620t). These components correspond to a portion of the wire 5605 extending along five complete turns of the wire path 5606. In other embodiments, as discussed above, counterparts of the proximal end portion 5602, the distal end portion 5603, and the intermediate portion 5604 can have other suitable delineations. Furthermore, these counterparts can include other suitable quantities and/or types of components.

In at least some cases, the wire 5605 is unbranched throughout the wire path 5606. For example, the wire 5605 can lack bifurcations, trifurcations, or other types of junctions at which the wire 5605 divides. In addition or alternatively, the wire 5605 can be untethered throughout the wire path 5606. For example, the wire 5605 can lack bridges or other structural connections between different portions of the wire 5605 spaced apart from one another along the wire path 5606 and/or between the wire 5605 and other implant components. By way of nonbinding theory, these features alone or in combination with other features described herein may be useful to reduce a foreign body response associated with the implant 5600, to increase longitudinal flexibility of the implant 5600, and/or for one or more other reasons. In other embodiments, a counterpart of the wire 5605 can be branched, tethered, and/or present with other implant components.

With reference again to FIGS. 18A-20, the first terminus 5609 and/or the second terminus 5610 can be untethered. In contrast, wire ends in conventional implants are typically tethered in some manner, such as by being tied or otherwise bonded to other wire portions. This tethering is intuitive because untethered wire ends are conventionally assumed to have greater potential than tethered wire ends to cause trauma, to migrate, and/or to exhibit other undesirable behaviors after implant deployment. With reference again to FIGS. 18A-19B, the inventors recognized that making the first terminus 5609 and/or the second terminus 5610 untethered had potential benefits and that associated problems could be mitigated or even eliminated with other implant features. Among the benefits is supporting mucociliary clearance. The inventors recognized that a lack of branching and/or tethering at other portions of the wire 5605 and/or the lack of structures of the implant 5600 other than the wire 5605, as discussed above, can also support this objective. Moreover, without wishing to be bound to this theory, the inventors identified mucociliary clearance as useful for supporting long-term use of the implant 5600 without loss of airway patency due to mucus impaction or the accumulation of granulation tissue. Accordingly, the implant 5600 can be configured to allow mucociliary clearance from a location immediately distal to the implant 5600 to a location immediately proximal to the implant 5600 while the implant 5600 is deployed at a treatment location within a bronchial tree.

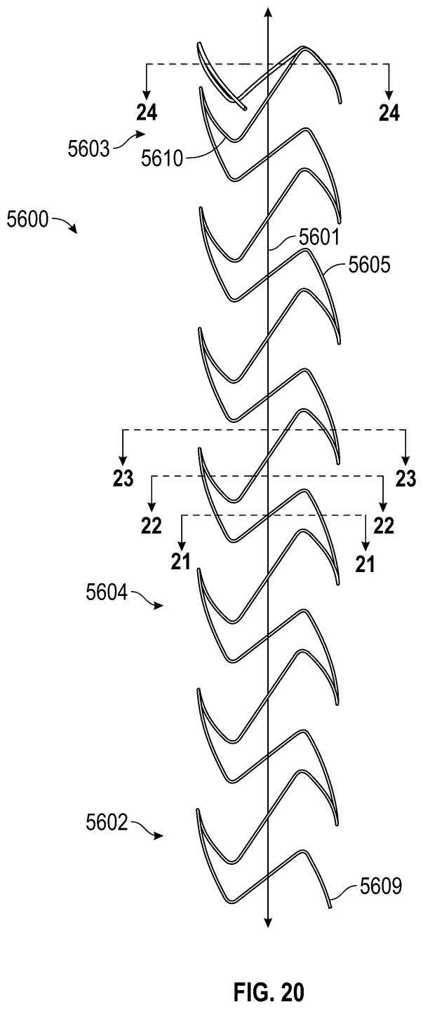

As best shown in FIG. 20, the first terminus 5609 can be at a proximalmost end of the implant 5600. Correspondingly, the implant 5600 can include a given one of the first legs 5614 at the first end 5607 of the wire path 5606. Furthermore, a pitch of the wire path 5606 at the proximal end portion 5602 can be about the same as (e.g., within 10% of) a pitch of the wire path 5606 at the intermediate portion 5604. These features and a lack of tethering at the first terminus alone or in combination can facilitate retrievability of the implant 5600. For example, although the implant 5600 is expected to be suitable for indefinite use, in some cases it may be useful to remove the implant 5600 from a treatment location after deployment. This may be the case, for example, when a clinician deploys the implant 5600 improperly or when unexpected and unusual biological processes cause an airway region in which the implant 5600 is deployed to eventually lose patency. Retrieving the implant 5600 can include gripping the wire 5605 at or near the first terminus 5609 and pulling the wire 5605 proximally. The described features of the first terminus 5609 can facilitate gripping access and can help guide the wire 5605 away from airway walls in response to pulling force. For example, the implant 5600 generally and the proximal end portion 5602 particularly can be configured to unwind and elongate rather than maintain the same shape perpendicular to the longitudinal axis 5601 during retrieval. Accordingly, rather than dragging across the airway walls proximally, the implant 5600 can tend to disengage inwardly and then move proximally during retrieval. This can reduce or eliminate excess trauma.

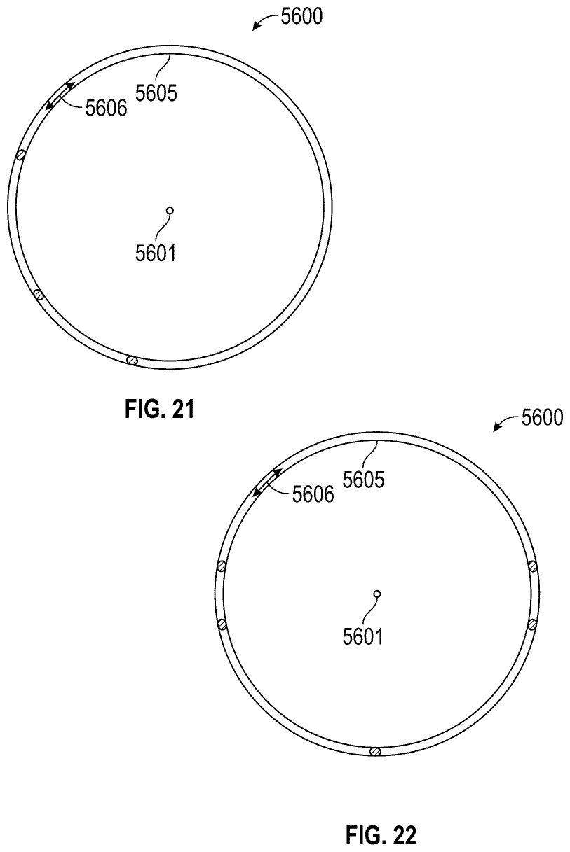

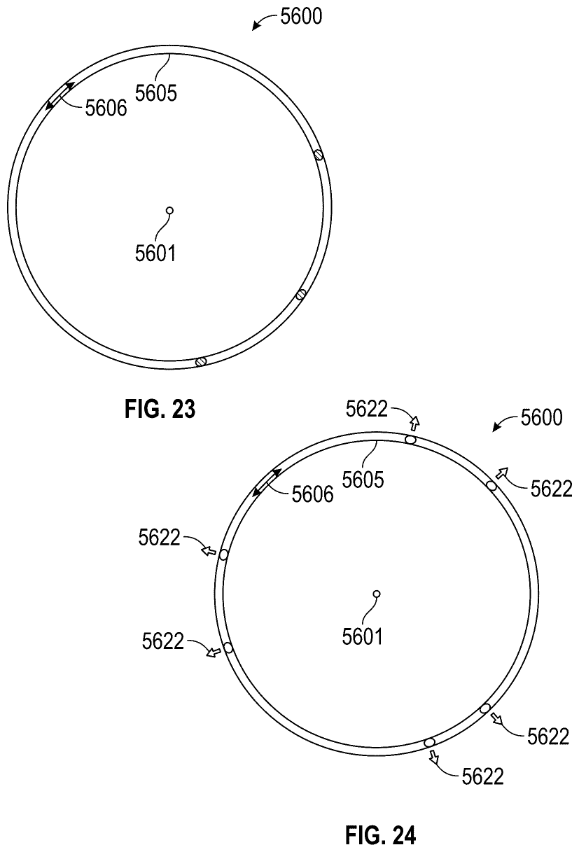

FIGS. 21, 22, 23 and 24 are cross-sectional views of the implant 5600 taken along lines A-A, B-B, C-C, and D-D in FIG. 20, respectively. As shown in FIGS. 21-62, planes perpendicular to the longitudinal axis 5601 at different portions of the implant 5600 can intersect more than one circumferentially spaced apart portion of the implant 5600. This contrasts with a simple coil. The inventors have discovered that contacting more than one circumferentially spaced apart portions of a wall of an airway region can be useful for establishing and maintaining airway patency. Portions of the implant 5600 that a plane perpendicular to the longitudinal axis 5601 intersects can correspond to portions of the implant 5600 that contact a wall of an airway region when the implant 5600 is deployed. Accordingly, as shown in FIGS. 20-24, the implant 5600 can contact three circumferentially spaced apart portions of a wall of an airway region at a plane perpendicular to the longitudinal axis 5601 at the line A-A, five such portions at the line B-B, three such portions at the line C-C, and six such portions at the line D-D. Lines A-A, B-B, and C-C are at the intermediate portion 5604 whereas line D-D is at the distal end portion 5603. In at least some cases, any given plane perpendicular to the longitudinal axis 5601 at the intermediate portion 5604 and/or a middle 50% of a length of the implant 5600 along the longitudinal axis 5601 intersects at least three (e.g., from three to five) circumferentially spaced apart points along the wire path 5606.

As FIGS. 21-24 suggest, the implant 5600 can be configured to contact more circumferentially spaced apart portions of a wall of an airway region at planes perpendicular to the longitudinal axis 5601 at the distal end portion 5603 than at planes perpendicular to the longitudinal axis 5601 at the intermediate portion 5604. For example, the implant 5600 can be configured to intersect at least a first number of circumferentially spaced apart points along the wire path 5606 at any given plane perpendicular to a middle 50% of a length of the implant 5600 along the longitudinal axis 5601 and to intersect at least a greater second number of circumferentially spaced apart points along the wire path 5606 at any given plane perpendicular to distalmost 5% of the length of the implant 5600 along the longitudinal axis 5601. In at least some cases, the second number of circumferentially spaced apart points is at least five. Furthermore, among the circumferentially spaced apart points along the wire path 5606 at which any given plane perpendicular to distalmost 5% of the length of the implant 5600 along the longitudinal axis 5601 intersects the implant, a maximum circumferential spacing between any circumferentially neighboring pair of the points can be no more than 180 degrees, such as no more than 120 degrees. Conversely, for at least one neighboring pair of circumferentially spaced apart points, there may be a minimum circumferential spacing of at least 60 degrees, such as at least 90 degrees, 120 degrees, or 150 degrees.

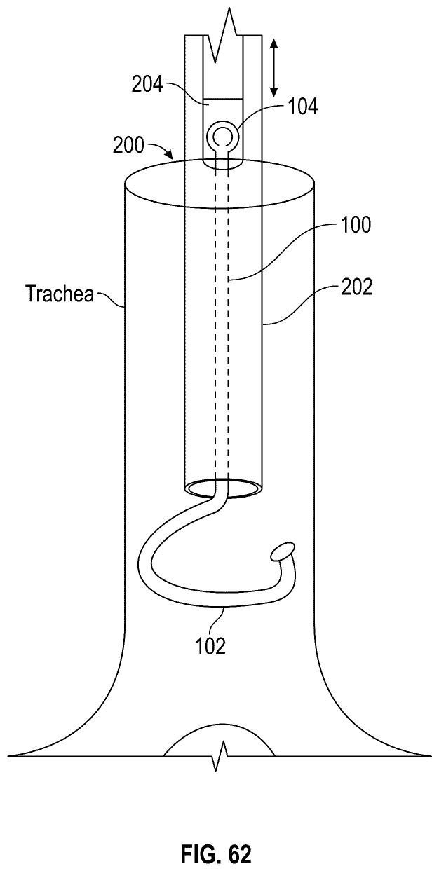

The inventors recognized a relatively large number of and/or relatively circumferentially balanced positioning of points of contact between the distal end portion 5603 and an airway region as potentially useful to facilitate deployment of the implant 5600. For example, in at least some cases, the implant 5600 is deployed by causing relative movement between a sheath and the implant 5600 such that the implant 5600 is gradually uncovered and allowed to expand radially. In these and other cases, the distal end portion 5603 can expand before other portions of the implant 5600. When this expansion begins, the distal end portion 5603 may have no established connection to the airway region. If a counterpart of the distal end portion 5603 initiated and/or propagated connection with an airway region at a single point, the force exerted against the airway region at that point would potentially cause asymmetrical expansion of the airway region. This, in turn, would potentially cause the counterpart of the distal end portion 5603 to move unpredictable during deployment, leading to potential trauma and/or suboptimal control over positioning. In contrast, with reference again to FIG. 62, the distal end portion 5603 can be configured to exert force (corresponding to arrows 5622) at a sufficient number of circumferentially spaced apart portions of the airway region to cause the airway region to expand relatively uniformly, thereby reducing potential trauma and/or enhancing control over positioning. After its deployment, the distal end portion 5603 can anchor the implant 5600 such that further radial expansion of the implant 5600 does not cause trauma or unduly compromise control over positioning of the implant 5600 even if such further expansion propagates along a relatively small number of points and/or points that are relatively circumferentially unbalanced.

The cross-sectional shape of the implants disclosed herein can comprise a radially symmetric circle, at least in an unconstrained, relaxed state. For example, in some embodiments, the implant 5600 can be heat set on a cylindrical mold. Such a design can be configured to apply a radially outward force equally in all radial directions, or in equal segments of the circumference, which may advantageously be useful for treating a wide variety of patients and ECAC morphologies. Any of the implants disclosed herein can have other cross-sectional shapes, such as a D or C shape, as discussed in greater detail below.

### Implant Geometry and Contact Density

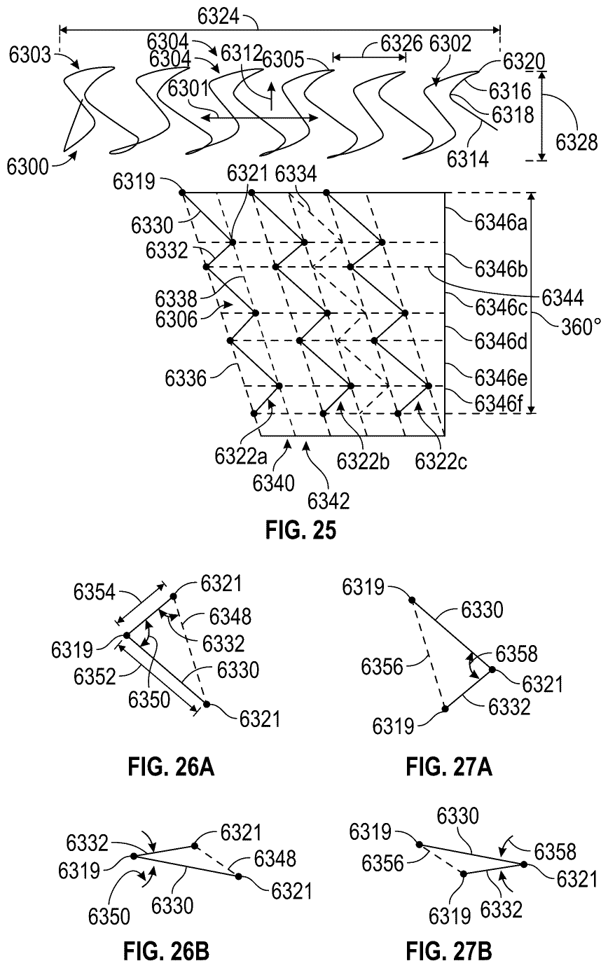

FIG. 25 is a profile view of an implant 6300 in accordance with at least some embodiments of the present technology in an unconstrained state juxtaposed with a schematic diagram illustrating certain geometrical aspects of the implant 6300. The implant 6300 is generally similar to the implant 5600 described above except that the implant 6300 has fewer turns and different wire termination features. With reference to FIGS. 18A-18F and 25 together, the implant 6300 can include or define a longitudinal axis 6301, a proximal end portion 6302, a distal end portion 6303, a intermediate portion 6304, a wire 6305, a wire path 6306, a circumferential direction 6312 (as indicated and curving into the page), first legs 6314, second legs 6316, first apex portions 6318, first apex points 6319, second apex portions 6320, and second apex points 6321 at least generally corresponding to the longitudinal axis 5601, the proximal end portion 5602, the distal end portion 5603, the intermediate portion 5604, the wire 5605, the wire path 5606, the circumferential direction 5612, the first legs 5614, the second legs 5616, the first apex portions 5618, the first apex points 5619, the second apex portions 5620, and the second apex points 5621, respectively, of the implant 5600.

With reference now to FIG. 25, the wire path 6306 is shown in a two-dimensional unwound representation with portions of the wire path 6306 corresponding to three successive turns 6322 (individually identified at turns 6322a-6322c) of the wire path 6306 at the intermediate portion 6304. The vertical axis in the schematic diagram corresponds to circumferential position and spacing in the circumferential direction 6312 about the longitudinal axis 6301. The horizontal axis in the schematic diagram corresponds to longitudinal position and spacing along the longitudinal axis 6301. The implant 6300 can define a length 6324 along the longitudinal axis 6301, a pitch 6326 along the longitudinal axis 6301, and a diameter 6328 perpendicular to the longitudinal axis 6301. In the schematic diagram, first segments 6330 of the wire path 6306 correspond to lengths of the first legs 6314. Similarly, second segments 6332 of the wire path 6306 correspond to lengths of the second legs 6316. For the sake of simplicity, the first and second segments 6330, 6332 are represented as straight lines between neighboring first and second apex points.

In the illustrated embodiment, the length 6324 is about 50 mm, the average pitch 6326 at the intermediate portion 6304 is about 8.1 mm, and the average diameter 6328 is about 10 mm. In other embodiments, these dimensions can be different. For example, a counterpart of the length 6324 can be within a range from 50 mm to 200 mm, such as from 70 mm to 200 mm or from 70 mm to 120 mm. Alternatively, a counterpart of the length 6324 can be less than 50 mm or greater than 200 mm. A counterpart of the average pitch 6326 at the intermediate portion 6304 can be within a range from 4 mm to 12 mm, such as from 6 mm to 12 mm, or from 6 mm to 10 mm. Alternatively, a counterpart of the average pitch 6326 can be less than 4 mm or greater than 12 mm. A counterpart of the average diameter 6328 can be within a range from 2 mm to 20 mm, such as from 4 mm to 20 mm, or from 5 mm to 15 mm. Alternatively, a counterpart of the average diameter 6328 can be less than 2 mm or greater than 20 mm. In other embodiments, counterparts of the implant 6300 can have still other suitable dimensions.

With reference again to the illustrated embodiment, the average pitch 6326 at the distal end portion 6303 can be smaller than the average pitch 6326 at the intermediate portion 6304 and smaller (e.g., from 10% to 50% smaller) than the average pitch 6326 at the proximal end portion 6302. This pitch difference can correspond to a greater number of circumferentially spaced apart portions of the wire 6305 along which contact between the implant 6300 and an airway wall simultaneously propagates during deployment of the distal end portion 6303 relative to deployment of the intermediate portion 6304. In addition or alternatively, this pitch difference can correspond to a greater degree of circumferential balance among portions of the wire 6305 along which contact between the implant 6300 and an airway wall simultaneously propagates during deployment of the distal end portion 6303 relative to deployment of the intermediate portion 6304. As discussed above, the number of contact portions and/or the circumferential balance of these contact portions can be useful to reduce potential trauma and/or enhance control over positioning during implant deployment.

The pitch 6326 can also be relevant to performance characteristics of the implant 6300, such as enhancing mucociliary clearance. In at least some cases, the implant 6300 is configured to define an unobstructed mucociliary clearance region extending along a continuous mucociliary clearance path 6334 from the location immediately distal to the implant 6300 to the location immediately proximal to the implant 6300 while the implant 6300 is deployed at a treatment location within a bronchial tree of a human subject. As shown in FIG. 25, the mucociliary clearance path 6334 can extend between successive turns of the wire path 6306. An average width of the mucociliary clearance region parallel to the longitudinal axis 6301 can be significantly greater than an average cross-sectional diameter of the wire 6305 perpendicular to the wire path 6306. This can correspond to a synergistic combination of relatively small contact area between the implant 6300 and an airway wall thereby a foreign body response and relatively large area available for mucociliary clearance. These features alone or together can increase the time (potentially indefinitely) during which an airway region in which the implant 6300 is deployed remains patent. In at least some cases, the average width of the mucociliary clearance region parallel to the longitudinal axis 6301 is at least 10 times (e.g., within a range from 10 times to 20 times) the average cross-sectional diameter of the wire 6305 perpendicular to the wire path 6306. In addition or alternatively, the average pitch 6326 can be within a range from 50% to 110% (e.g., from 70% to 90%) of the average diameter 6328. This can be the case, for example, at the intermediate portion 6304 and/or throughout the implant 6300.

The implant 6300 can be configured to resiliently transition from a low-profile delivery state to an expanded deployed state. The average diameter 6328 can be significantly different between these states. By way of nonbinding theory, the inventors have found that this feature has great potential to facilitate establishing and maintaining airway patency. Expansion of an airway well beyond its native diameter creates a relatively large free-passage area that is less likely or at least slower to become occluded due to mucus impaction or the accumulation of granulation tissue. In some embodiments, the average diameter 6328 when the implant 6300 is in the deployed state is at least 3 times (e.g., at least 3.5 times, at least 4 times, at least 4.5 times, or at least 5 times) the average diameter 6328 when the implant 6300 is in the delivery state. In these and other embodiments, the average diameter 6328 when the implant 6300 is in the illustrated unconstrained state is at least 4 times (e.g., at least 4.5 times, at least 5 times, at least 5.5 times, or at least 6 times) the average diameter 6328 when the implant 6300 is in the delivery state. Furthermore, a ratio of the average diameter 6328 to the length 6324 can be within a range from 1:5 to 1:30, such as from 1:10 to 1:30.

In the illustrated embodiment, the diameter 6328 is consistent throughout the length 6324. In at least some cases, the diameter 6328 varies no more than 5% or no more than 10% throughout the length 6324. Relatedly an average of the diameter 6328 at the proximal end portion 6302 can be no more than 5% different or no more than 10% different than an average of the diameter 6328 at the distal end portion 6303. This may be counterintuitive because the distal end portion 6303 is configured to be deployed at a more distal portion of a bronchial tree than the portion at which the proximal end portion 6302 is deployed. More distal airway regions of a bronchial tree are typically narrower than more proximal portions. Having the diameter 6328 be relatively consistent throughout the length 6324 can be beneficial, however, for establishing and/or maintaining airway patency. For example, it may be beneficial for a degree of relative hyper-expansion of a wall of an airway region to be greater distally than proximally. This is expected to follow from deployment of a consistent diameter implant in a distally narrowing airway region. Other advantages are also possible. Furthermore, in other embodiments, a counterpart of the diameter 6328 may be inconsistent along a counterpart of the length 6324. For example, a counterpart of the diameter 6328 may increase or decrease along the counterpart of the length 6324. In these cases, an average counterpart diameter 6328 of a counterpart proximal end portion 6302 can be smaller or larger than an average counterpart diameter 6328 of a counterpart distal end portion 6303.

With reference again to FIG. 25, the first apex portions 6318 at the intermediate portion 6304 can define a first helix 6336. Similarly, the second apex portions 6321 at the intermediate portion 6304 can define a second helix 6338. In at least some cases, the longitudinal axis 6301 is an axis of symmetry about which the first and second helixes 6336, 6338 are wound. The implant 6300 can define a first helical band 6340 between the first helix 6336 and the second helix 6338. In the illustrated embodiment, successive turns of the first helical band 6340 are spaced apart from one another along the longitudinal axis 6301 such that the implant 6300 defines a second helical band 6342 intertwined with the first helical band 6340. In at least some cases, an average width of the first helical band 6340 is within a range from 30% to 75% of the average pitch 6326 at the intermediate portion 5604 when the implant 6300 is in the deployed state. As the implant 6300 transitions from the delivery state toward the deployed state or the unconstrained state, the average width of the first helical band 6340 parallel to the longitudinal axis 6301 can decrease and an average width of the second helical band 6342 parallel to the longitudinal axis 6301 can increase. Conversely, as the implant 6300 transitions from the deployed state or the unconstrained state toward the delivery state, the average width of the first helical band 6340 parallel to the longitudinal axis 6301 can increase and the average width of the second helical band 6342 parallel to the longitudinal axis 6301 can decrease.

In some cases, it is useful for the second helical band 6342 to still be present when the implant 6300 is in the delivery state. Stated differently, in these cases, it can be useful for successive turns of the first helical band 6340 to be spaced apart from one another along the longitudinal axis 6301 when the implant 6300 is in the delivery state. This can be useful, for example, to reduce or eliminate overlapping of the wire path 6306 when the implant 6300 is in the delivery state. Overlapping of the wire path 6306 can cause the implant 6300 to be less compact in the delivery state than would otherwise be the case. This can be disadvantageous as it may reduce an ability of the implant 6300 to be delivered intraluminally to more distal airways. In other cases, a counterpart of the second helical band 6342 may be eliminated when a counterpart of the implant 6300 is in a delivery state. Stated differently, in these other cases, successive turns of a counterpart of the first helical band 6340 may be overlapping when the counterpart of the implant 6300 is in the delivery state. The circumferential alignment of features within a counterpart of the first helical band 6340 between successive turns thereof can affect whether a counterpart of the wire path 6306 does or does not overlap in these cases. When the circumferential alignment of these features is such that a counterpart of the wire path 6306 does not overlap, then overlapping a counterpart of the first helical band 6340 when a counterpart of the implant 6300 is in a delivery state may be advantageous. For example, via nesting or interdigitation, this overlapping may allow more longitudinally expansive structures to be present in the same longitudinal space. As discussed below, however, circumferential alignment of features within the first helical band 6340 has other implications which may outweigh, conflict with, or be complementary with this potential advantage.

As shown in FIG. 25, a given three of the first apex points 6319 and the corresponding first apex portions 6320 at respective neighboring turns 6322 of the wire path 6306 at the intermediate portion 6304 can be circumferentially aligned with one another. For example, the given three of the first apex points 6319 and the corresponding first apex portions 6320 can be within 5 degrees or within 10 degrees of circumferential alignment with one another. Furthermore, this circumferential alignment can be present for one, some, or all of the first apex points 6319 and the corresponding first apex portions 6320 at the neighboring turns 6322. The lines 6344 in FIG. 25 indicate this circumferential alignment. In at least some cases, the circumferential alignment in the stated ranges persists as the implant 6300 transitions between the delivery state and the deployed state or between the delivery state and the unconstrained state. Accordingly, the given three of the first apex points 6319 and the corresponding first apex portions 6320 at the respective neighboring turns 6322 of the wire path 6306 at the intermediate portion 6304 can be circumferentially aligned with one another when the implant 6300 is in the delivery state, the deployed state, and the unconstrained state. By way of nonbinding theory, this persistence of circumferential alignment may have certain advantages, such as reducing or eliminating a tendency of the implant 6300 to shift after deployment at a treatment location. Such shifting may increase a foreign body response, increase airway erosion, and/or have other undesirable effects.

In FIG. 25, line segments 6346 represent circumferential spacing between successive apex points among the first and second apex points 6319, 6321 along the wire path 6306 at the intermediate portion 6304. In at least some embodiments, an average of this circumferential spacing is within a range from 35 degrees to 95 degrees, such as from 55 degrees to 65 degrees. As with the circumferential alignment, the average circumferential spacing can persist as the implant 6300 transitions between the delivery state and the deployed state or between the delivery state and the unconstrained state. In at least some cases, the average circumferential spacing between successive apex points among the first and second apex points 6319, 6321 along the wire path 6306 at the intermediate portion 6304 when the implant 6300 is in the delivery state is no more than 5% or no more than 10% different than when the implant 6300 is in the deployed state. Similarly, this average circumferential spacing when the implant 6300 is in the delivery state can be no more than 5% or no more than 10% different than when the implant 6300 is in the unconstrained state. By way of nonbinding theory, this persistence of circumferential spacing may have certain advantages similar to the advantages discussed above with regard to the persistence of circumferential alignment.

FIGS. 26A-65B are diagrams showing different respective subtended angles relevant to the implant 6300. In particular, FIGS. 26A and 26B illustrate a portion of the wire path 6306 corresponding to a given one of the first segments 6330 (corresponding to a given one of the first legs 6314) and a given one of the second segments 6332 (corresponding to a given one of the second legs 6316) at opposite sides of a given one of the first apex points 6319 when the implant 6300 is in the unconstrained state and the delivery state, respectively. Similarly, FIGS. 27A and 65B illustrate a portion of the wire path 6306 corresponding to a given one of the first segments 6330 and a given one of the second segments 6332 at opposite sides of a given one of the second apex points 6321 when the implant 6300 is in the unconstrained state and the delivery state, respectively. As shown in FIG. 26A, a first line 6348 between a pair of the first apex points 6319 neighboring one another along the wire path 6306 subtends a first angle 6350 from an intervening one of the second apex points 6321 along the wire path 6306. FIG. 26A also illustrates a length 6352 of the given first segment 6330 and a length 6354 of the given second segment 6332 at opposite sides of the given first apex point 6319. As shown in FIG. 27A, a second line 6356 between a pair of the second apex points 6321 neighboring one another along the wire path 6306 subtends a second angle 6358 from an intervening one of the first apex points 6319 along the wire path 6306. In at least some cases, one or both of the first and second angles 6350, 6358 are within a range from −20 degrees to 20 degrees (e.g., from −20 degrees to 10 degrees) when the implant 6300 is in the delivery state and within a range from 20 degrees to 90 degrees (e.g., from 40 degrees to 90 degrees) when the implant 6300 is in the deployed state. This angle can be negative when segments of the wire path 6306 at opposite sides of an apex point converge and then diverge as they extend away from the apex point.

An average length 6352 of the first legs 6314 at the intermediate portion 6304 can be different than an average length 6354 of the second legs 6316 at the intermediate portion 6304. For example, the average length 6352 of the first legs 6314 at the intermediate portion 6304 can be greater than (e.g., from 20% to 50% greater than) an average length 6354 of the second legs 6316 at the intermediate portion 6304. Furthermore, a ratio of the average length 6352 of the first legs 6314 at the intermediate portion 6304 to the average length of the second legs 6316 at the intermediate portion 6304 can be greater than a threshold value of n/(n−1) with n being an average number of the first legs 6314 per complete turn 6322 of the wire path about the longitudinal axis at the intermediate portion. For example, the ratio of the average length 6352 of the first legs 6314 at the intermediate portion 6304 to the average length of the second legs 6316 at the intermediate portion 6304 can be within a range from 80% to 99% of the threshold value. This may facilitate avoiding overlap of the wire path 6306 when the implant 6300 is in the delivery state without unduly compromising a degree to which the implant supports an airway region and inhibits invagination of a wall of the airway region.

The implant 6300 can have a surprisingly small airway contact density. In general the amount of force needed to expand an airway region wall is relatively independent of the amount of contact between an implant and the airway region wall. Accordingly, smaller airway contact density corresponds to a need for greater force density. The inventors discovered that airways in a human bronchial tree are capable of withstanding surprisingly high force densities. Accordingly, airway contact density can be reduced without unduly compromising performance. Furthermore, low contact density is expected to have beneficial impacts on maintaining airway patency. For example, low contact density is expected to reduce foreign body response and facilitate mucociliary clearance. Moreover, high force density may actually be beneficial by increasing stability as further discussed below. Airway-to-implant contact density is expected to correspond to the following Equation 1 (Eq. 1):

$$\frac{{A}_{iw}}{{A}_{i}} = \frac{n \cdot ( {l}_{s} + {l}_{l} ) \cdot {d}_{w}}{2 \cdot {d}_{a} \cdot {l}_{p}} \qquad ( Eq . 1 )$$

$${A}_{i} = area supported by a single turn$$

$${A}_{iw} = area of a single turn$$

$${d}_{w} = diameter of implant$$

$${d}_{a} = diameter of airway$$

$$n = number of implant bends per turn$$

In at least some embodiments, the implant 6300 is configured to occupy from 5% to 30%, such as from 5% to 15%, of a total area of the first helical band 6340 when the implant 6300 is in the deployed state.

### Implant Stability

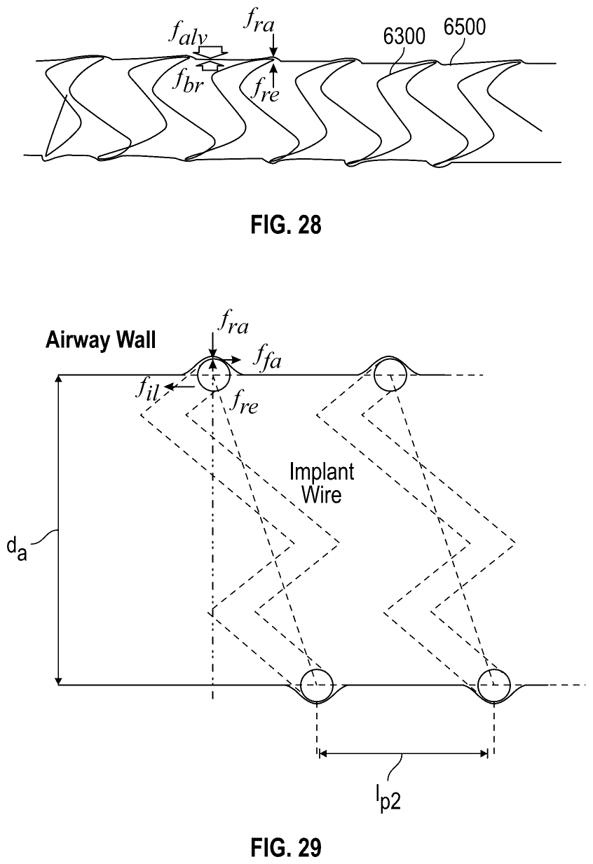

FIG. 28 is a profile view of the implant 6300 in a deployed state within an airway region 6500. In this state, radial forces on the implant 6300 and on the airway region 6500 are expected to be in balance in accordance with the following Equation 2 (Eq. 2):

$${f}_{ra} + {f}_{alv} = {f}_{re} + {f}_{br} \qquad ( Eq . 2 )$$

$${f}_{re} = force of radial expansion of a single wire$$

$${f}_{ra} = force of reaction of airway of a single wire$$

$${f}_{alv} = force applied by alveolar presure to a single wire turn$$

$${f}_{br} = force applied by brochial presure to a single wire turn$$

The diameter 6328 and the radial spring constant of the implant 6300 can be selected in view of the following Equation 3 (Eq. 3):

$${f}_{re} = {k}_{ir} ( {d}_{in} - {d}_{a} ) = {k}_{ar} ( {d}_{an} - {d}_{a} ) + π \cdot {d}_{a} \cdot {l}_{p} \cdot ( {P}_{alv} - {P}_{br} ) \qquad ( Eq . 3 )$$

$${f}_{re} = force of radial expansion of a single wire$$

$${k}_{ir} = spring constant of implant in radial direction$$

$${k}_{ar} = spring constant of airway in radial direction$$

$${d}_{in} = nominal diameter of implant$$

$${d}_{a} = diameter of airway$$

$${d}_{an} = nominal diameter of airway$$

$${l}_{p} = implant pitch length$$

$${P}_{alv} = pressure in the alveoli$$

$${P}_{br} = pressure in the bronchi$$

As discussed above, the inventors discovered that airways in a human bronchial tree are capable of withstanding surprisingly high force densities and that high force densities may be beneficial to enhance implant stability and/or for other reasons. Accordingly, the diameter to which the implant 6300 is configured to expand an airway can be many times greater (e.g., at least 2 times, 2.5 time, 3 times, 3.5 times, or 4 times greater) than a nominal diameter of the airway.

Stable contact between an implant and an airway wall can be challenging to achieve for at least two reasons. First, relevant airway regions are typically tortuous, branched, and/or of widely varying diameter. Second, these airway regions typically move significantly and nonuniformly during respiration, coughing, sneezing, etc. Relative movement between an airway region and an implant can cause or contribute to irritation, erosion, foreign body response, and/or other factors that tend to decrease long-term patency. Together with or instead of high force density, the inventors recognized that relatively low resistance to longitudinal deformation together with relatively high resistance to radial deformation can enhance implant stability.

FIG. 29 is a schematic diagram illustrating certain forces and dimensions relevant to implants in accordance with at least some embodiments of the present technology. In FIG. 66, two neighboring turns of an implant 6600 are shown in a deployed state in an airway region 6602. Both radial and longitudinal forces are identified. In at least some cases, when the force of the implant 6600 reacting to elongation/shortening is less than the force of friction on the implant 6600, the implant 6600 tends to remain stable during breathing. The radial and longitudinal spring constants of the implant 6600 can be selected in accordance with the following Equation 4 (Eq. 4):

$$\frac{{k}_{il}}{{k}_{ir}} \leq {μ}_{a - i} \frac{( {d}_{in} - {d}_{a} )}{( {l}_{pn} - {l}_{p 2} )} \qquad ( Eq . 4 )$$

$${k}_{ir} = spring constant of implant in radial direction$$

$${k}_{il} = spring constant of implant in longitudinal direction$$

$${μ}_{a - i} = coefficient of friction between airway and spring$$

$${d}_{a} = diameter of airway$$

$${d}_{in} = nominal diameter of implant$$

$${l}_{pn} = nominal implant pitch$$

$${l}_{p 2} = distance between adjacent turns with lung motion$$

Implants in accordance with at least some embodiments of the present technology have a ratio of radial spring constant to longitudinal spring constant within a range from 10:1 to 80:1, such as from 15:1 to 80:1 or from 20:1 to 80:1.

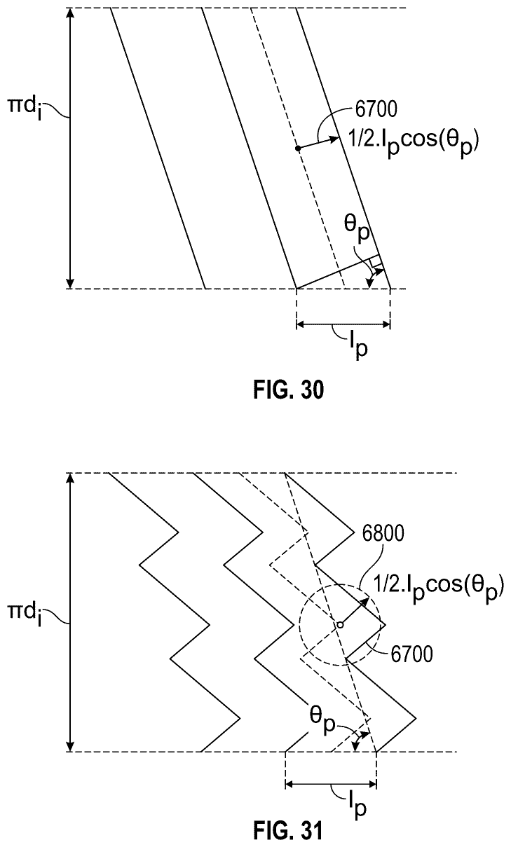

A wire including alternating first and second legs can support and airway to a greater extent than a wire shaped as a simple coil even if both wires have the same pitch. FIG. 30 is a schematic diagram illustrating a maximum distance between a point on an airway wall and a wire path of a simple coil. FIG. 31 is a schematic diagram illustrating a maximum distance between a point on an airway wall and a wire path of an implant in accordance with at least some embodiments of the present technology. The maximum distance in FIG. 30 is represented by line 6700 and can be calculated using the following Equation 5 (Eq. 5):

$$maximum distance = \frac{1}{2} \cdot {l}_{p} cos {θ}_{p} \qquad ( Eq . 5 )$$

$${l}_{p} = implant pitch length$$

$${θ}_{p} = implant pitch angle$$

In FIG. 31, a circle 6702 having a radius equal to the length of the line 6700 is centered on a point along a line midway between neighboring turns of the wire path. The circle overlaps the wire path indicating that a portion of an airway at the point is closer to the wire and thus better supported with the wire path of FIG. 31 and with the wire path of FIG. 30.

Another implant feature the inventors recognized as potentially relevant to maintaining stable contact between an implant and an airway wall during respiration is resistance to flattening from a tubular form toward a more planar form. Some tubular structures have longitudinally distributed substructures (e.g., helical turns) that easily domino or otherwise collapse on one another in response to shear stress parallel to the structures' longitudinal axes. This is problematic because this type of shear stress may occur in airways during respiration. In contrast to blood vessels that expand and contract to a limited extent and primarily radially rather than longitudinally during pulsatile blood flow, airways during respiration expand and contract far more significantly and do so both radially and longitudinally. Accordingly, achieving an adequate resistance to flattening can be far more challenging in the context of pulmonary implants than in the context of vascular implants. Due to the structural features discussed below and/or for other reasons, implants in accordance with at least some embodiments of the present technology are well suited to resisting flattening. For example, implants in accordance with at least some embodiments of the present technology have a ratio of radial spring constant to longitudinal shear modulus suitable for resisting flattening. This ratio, for example, can be within a range from 0.005 to 0.100. In addition or alternatively, implants in accordance with at least some embodiments of the present technology have a ratio of longitudinal spring constant to longitudinal shear modulus suitable for resisting flattening. This ratio, for example, can be within a range from 0.5 to 5.0.

The above and/or other properties that promote stable wall contact during respiration can be related to certain structural features of implants in accordance with at least some embodiments of the present technology. One such feature is the complete or relative absence of stiff bridges between successive helical turns or other longitudinally distributed implant substructures. This feature can promote relatively low resistance to longitudinal deformation together with relatively high resistance to radial deformation, which, as discussed above, tends to promote stable contact between an implant an airway wall during respiration. This feature can also increase the tendency of an implant to flatten from a tubular form toward a more planar form, which, as also discussed above, can have the opposite effect. The inventors discovered, however, that the latter effect can be at least partially mitigated by increasing the average spacing (e.g., pitch) between successive helical turns or other longitudinally distributed implant substructures. Furthermore, both the complete or relative absence of stiff bridges between successive helical turns or other longitudinally distributed implant substructures and the increased spacing between these substructures synergistically help to maintain improved airway patency. Both of these features tend to facilitate mucociliary clearance and/or to reduce foreign body response. Implants in accordance with at least some embodiments of the present technology include longitudinally distributed substructures (e.g., helical turns) within a first helical band extending around a longitudinal axis and define an unobstructed second helical band between windings of the first helical band. In at least some cases, this feature is present together with a ratio of pitch to diameter within a range from 0.3:1 to 1.5:1, such as from 0.5:1 to 1.2:1.

### Implant Deployment

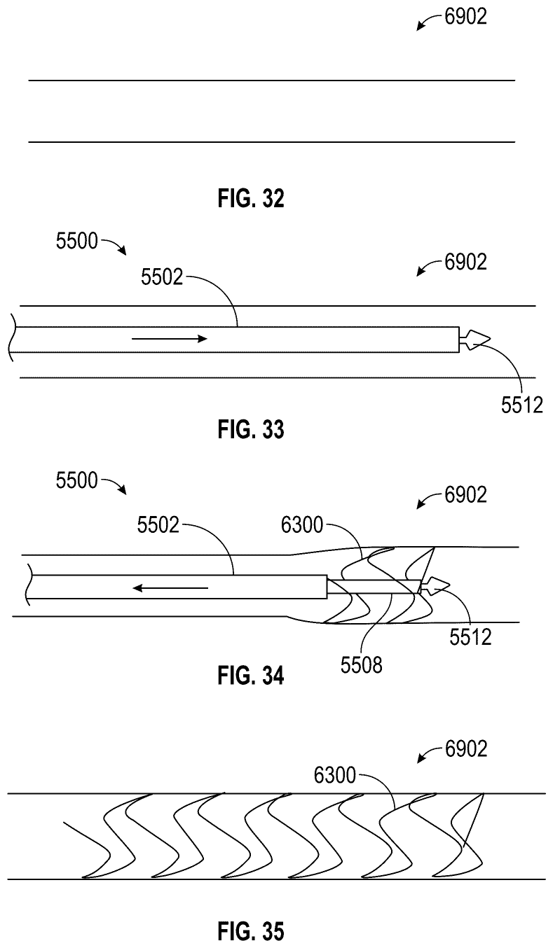

FIG. 32 is an anatomical illustration of an airway region 6902 of a human subject. The airway region 6902 may be, for example, a trachea or a left or a right main bronchus. FIGS. 70-75 are partially schematic illustrations of different respective times during deployment of an implant at the airway region 6902. This deployment will now be described primarily with respect to the implant 6300 (FIG. 25) and the delivery system 5500 (FIG. 17A). It should be understood, however, that the deployment can be practiced with any suitable implant or delivery system described herein, including with the assistance of a robotic system. Furthermore, the implant 6300 and other implants described herein can be compatible with other suitable types of deployment. With reference to FIGS. 17A, 25 and 32-75 together, the implant 6300 can be moved intraluminally within the bronchial tree toward a treatment location at the airway region 6902. For example, the implant 6300 can be coupled to an instrument driver of a robotic system and rotated, translated, and/or articulated within the bronchial tree 6904 via the driver.

Movement of the implant 6300 toward the treatment location can occur while the implant 6300 is in the low-profile delivery state. For example, the inner sheath 5508 can extend around the implant 6300 and constrain radial expansion of the implant 6300 during this intraluminal movement. As shown in FIGS. 32 and 33, the delivery system 5500 can be moved distally (for example, manually or via robotic assistance) until the tip 5512 reaches a distal end of the diseased region. In some cases, as described above in the context of FIG. 17A, the delivery system 5500 can be deployed via a working channel of a bronchoscope. In these cases, a distal end of the bronchoscope (rather than the tip 5512) may interact with anatomy to limit a degree to which the implant 6300 can be advanced distally within the airway. In these cases, a camera of the bronchoscope can be used to guide positioning of the implant 6300. Movement of the bronchoscope and/or camera can be controlled manually or via a robotic system.

Once suitably located, the implant 6300 can be transitioned from the delivery state to the expanded deployed state at the treatment location (e.g., manually or via robotic assistance). As shown in FIG. 34, this can include causing relative movement between the implant 6300 and the inner sheath 5508. For example, the inner sheath 5508 can be retracted (e.g., manually or via robotic assistance) to expose the implant 6300 progressively beginning with a distalmost portion of the implant 6300 and moving proximally Exposing the implant 6300 can allow the implant to self-expand. For example, exposing the implant 6300 can release at least some resilient bias of the implant 6300 until the implant 6300 assumes an equilibrium state at which outward radial force from the implant 6300 equals inward radial force from the airway region 6902. In at least some cases, the implant 6300 is more resiliently biased at the first and second apex portions 6318, 6320 than at the first and second legs 6314, 6316 (see FIG. 25). Accordingly, the implant 6300 can be considered to include springs at the first and second apex portions 6318, 6320 and connectors at the first and second legs 6314, 6316. In other embodiments, the springs and connectors can have other suitable forms. Furthermore, the springs may be replaced with non-resilient expandable structures configured to expand via a mechanism (e.g., a balloon or other secondary structure within the implant 6300) other than resilience.

During relative movement between the implant 6300 and the inner sheath 5508, the proximal stop 5504 (FIG. 17A) can inhibit proximal movement of the overall implant 6300 and the conformable member 5510 can inhibit proximal movement of individual turns of the implant 6300. Thus, the implant 6300 can be deployed in a controlled manner to at least generally retain its longitudinal positioning and configuration as it expands radially. In at least some cases, the length 6324 of the implant 6300 is about the same (e.g., no more than 5% or 10% different) immediately after transitioning the implant 6300 relative to while the implant 6300 is still within the inner sheath 5508. Transitioning the implant 6300 can begin with expanding the distal end portion 6303 at the second airway 6908. This can include contacting a wall of the second airway 6908 and an untethered terminus of the wire 6305 at a portion of the wall of the second airway 6908 proximal to a distalmost end of the implant 6300. Expanding the distal end portion 6303 at the second airway 6908 can also include contacting the wall of the second airway 6908 and a given one of the second legs 6316 at an end of the wire path 6306. Transitioning the implant 6300 can proceed with expanding the intermediate portion 6304 and then expanding the proximal end portion 6302 at the first airway 6906. Expanding the proximal end portion 6302 at the first airway 6906 can include contacting a wall of the first airway 6906 and an untethered terminus of the wire 6305 at a portion of the wall of the first airway 6906 at a proximalmost end of the implant 6300. Expanding the proximal end portion 6302 at the first airway 6906 can also include contacting the wall of the first airway 6906 and a given one of the first legs 6314 at an end of the wire path 6306.

In at least some cases, during some (e.g., at least 50% or 75% by change in the diameter 6328) or all of expansion of the implant 6300 at the treatment location, an average degree of curvature of the wire path 6306 (FIG. 25) at the first and second apex portions 6318, 6320 increases, a width of the first helical band 6340 parallel to the longitudinal axis 6301 decreases, a helical length of the first helical band 6340 increases, a width of the second helical band 6342 parallel to the longitudinal axis 6301 increases, a given three of the first apex portions 6318 at respective neighboring turns 6322 of the wire path 6306 remain within 5 degrees of circumferential alignment with one another, a given three of the second apex portions 6320 at respective neighboring turns 6322 of the wire path 6306 remain within 5 degrees of circumferential alignment with one another, an average circumferential spacing between successive apex points among the first and second apex points 6319, 6321 collectively along the wire path 6306 remains within a range from 35 degrees to 95 degrees, the average circumferential spacing between the successive apex points remains within a range from 55 degrees to 65 degrees, and/or the average circumferential spacing in degrees between the successive apex points changes by no more than 5%.

As shown in FIG. 35, transitioning the implant 6300 can free the implant from the conformable member 5510 (see FIG. 17A). The conformable member 5510 can then be withdrawn proximally along with other portions of the delivery system 5500, thereby leaving the implant 6300 in the deployed state at the treatment location. Immediately after transitioning the implant 6300, the implant 6300 can exert a force against a wall of the airway of, for example, at least 0.05 megapascals. The airway region 6902 may be extremely flexible such that transitioning the implant 6300 expands a wall portion of the airway coextensive with the length of the implant 6300 well beyond a native diameter of this wall portion. In some embodiments, the implant expands the airway to a diameter greater than the diseased diameter, but not necessarily greater than the native diameter. Furthermore, the average diameter of the implant 6300 in the deployed state can be the same as or similar to (e.g., from 70% to 100% or from 80% to 100%) the average diameter of the implant 6300 in the unconstrained state. In addition or alternatively, a ratio of an average of the diameter of the implant 6300 immediately after transitioning the implant 6300 and the length of the implant 6300 immediately after transitioning the implant 6300 can be within a range from 1:5 to 1:15.

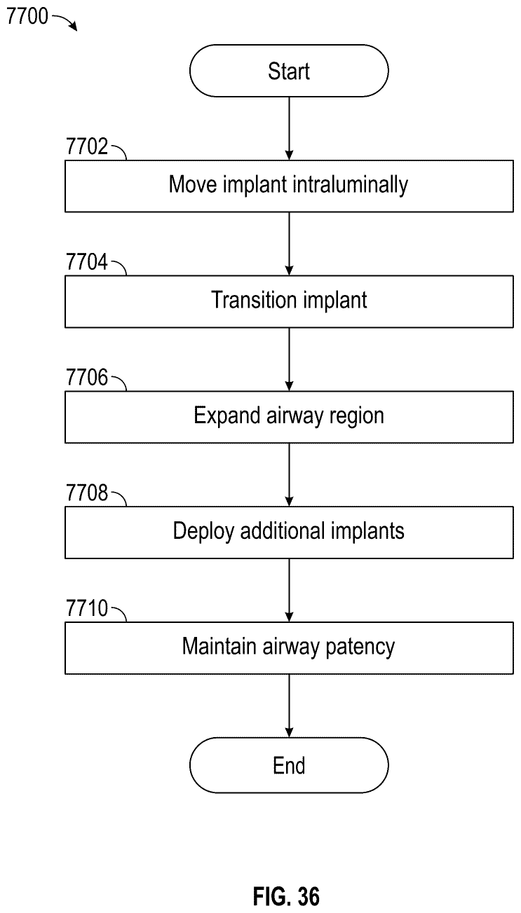

FIG. 36 is a block diagram showing a method 7600 for improving pulmonary function in a human subject in accordance with at least some embodiments of the present technology. In at least some cases, the subject is diagnosed with ECAC, such as TBM or EDAC. As shown in FIG. 36, the method 7600 can include moving an implant intraluminally within a bronchial tree of the subject toward a treatment location within the bronchial tree while the implant is in a low-profile delivery state (block 7602) (e.g., manually or via robotic assistance), transitioning the implant from the delivery state to an expanded deployed state at the treatment location (block 7604) and expanding an airway region at the treatment location (block 7606) (e.g., manually or via robotic assistance). These portions of the method 7600 are discussed in detail above in connection with implant deployment. The method 7600 can further include deploying additional implants (block 7608) (e.g., manually or via robotic assistance). For example, the deployment process described above with reference to FIGS. 32-35 can be repeated with additional implants at different respective airway regions. These airway regions, for example, can be associated with different pulmonary bullae. Deployment of the initial and subsequent implants can release trapped air and reduce or prevent further trapping of air at these pulmonary bullac.

Although not shown in FIG. 36, the method 7600 in some cases can include further modifying the airway region at which a given implant is deployed after deployment of the implant. When a treatment includes deploying multiple implants, this further modification can occur at one, some, or all of the treatment locations. As discussed above with reference to FIGS. 32-75, deploying the implant can expand a wall of an airway region to a first average expanded diameter. Further modification can include subsequently further expanding the wall to a second average expanded diameter larger than the first average expanded diameter. The balloon can be advanced intraluminally to the treatment location with the implant or after the implant is deployed and the delivery system removed. At the treatment location, the balloon can be expanded to cause both the wall and the implant to expand to the larger second average expanded diameter. In at least some cases, the second average expanded diameter is greater than an average unconstrained diameter of the implant.

In some cases, deploying one implant may be sufficient. In other cases, 2, 3, 4, 5, 6, or even greater numbers of implants may be deployed. Furthermore, one, two or another suitable first quantity of implants may be deployed at one time and one, two or another suitable second quantity of implants may be deployed at a second time hours, days, months or even longer after the first time. In a particular example, a first quantity of implants is deployed, followed by gathering monitoring, testing, and/or patient-reported information during a test period, and then a second quantity of implants is deployed based on a degree to which the first quantity of implants was effective in treating ECAC symptoms according to the information.

Deploying an implant at a treatment location can cause the treatment location to go from being low patency or nonpatent to having therapeutically effective patency. With reference to FIGS. 32-36 together, the method 7600 can include maintaining a therapeutically effective increase in patency at the treatment location throughout a continuous maintenance period while the implant 6300 is in the deployed state at the treatment location. The maintenance period can be at least 3 months, 6 months, 9 months, or another suitable period. During the maintenance period, a first area of a wall portion of the airway coextensive with the length of the implant 6300 along the longitudinal axis can be in direct contact with the implant 6300 and a second area of the wall portion can be out of direct contact with the implant 6300. The second area can be at least 5, 8, 10, 12, 14 or more times larger than the first area. In addition or alternatively, the wire 6305 can occupy from 5% to 30% (e.g., from 5% to 15%) of a total area of the first helical band 6340 during the maintenance period. Furthermore, a maximum invagination of the wall portion at the second area can be no more than 50% of the average expanded diameter of the implant 6300 during the maintenance period. Maintaining airway patency can also include maintaining a mucociliary clearance region at the treatment location substantially free of granulation tissue and mucoid impaction throughout the maintenance period. In addition or alternatively, maintaining airway patency includes maintaining the mucociliary clearance region substantially free of one some or all of inflammation, inflammatory cells, granulation tissue, fibrosis, fibrotic cells, tissue hyperplasia, tissue necrosis, granulation tissue, and mucoid impaction. The mucociliary clearance region can extend along a continuous mucociliary clearance path from a location immediately distal to the implant 6300 to a location immediately proximal to the implant 6300. In at least some cases, the mucociliary clearance region is maintained at an average width parallel to the longitudinal axis 6301 at least 10, 12, 14, 16 or more times greater than an average cross-sectional diameter of the wire 6305 perpendicular to the wire path 6306.

Part of maintaining airway patency can be reducing or eliminating excessive shifting of the implant 6300 during respiration. Relatedly, maintaining patency can include resisting elongation of the implant 6300 along the longitudinal axis during a full respiration cycle by the subject with a resisting force less than a force of friction between the implant 6300 and a wall of the airway at the treatment location. This feature alone or together with other features can reduce or prevent airway irritation and associated formation of granulation tissue and/or other response that may reduce airway patency during the maintenance period. In at least some cases, the implant maintains airway patency and/or other desirable therapeutic performance levels described herein during the maintenance period without the presence of a drug-eluting material between expandable structures of the implant and a wall of the airway at the treatment location.

The implants of the present technology can have a circular, substantially radially symmetric cross-sectional shape to apply radial force substantially equally in all radial directions, or in equal segments of the circumference. This may be beneficial, for example, as it eliminates the need to deploy the implant in a particular orientation, and also for treating a wide variety of patient morphologies. Such a design may be especially beneficial for treating infants, as infant tracheas may be shaped and behave similarly to bronchi (e.g., rounder in cross section, have an inner diameter that is similar, and collapse forces that are similar). Single-wire designs may also aid implant removal as the infant's anatomy changes over time and may require larger implants over time. In some embodiments, the O-shape can be made by a back-and-forth wire form that overlaps at the ends instead of a helical form.

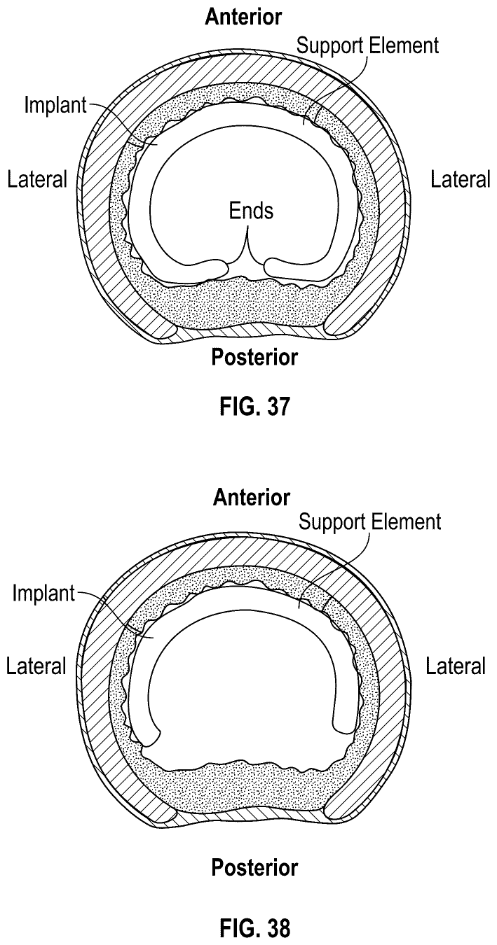

According to several embodiments, the implant can comprise a C-shaped design. For example, as shown in the cross-sectional view of FIG. 37, the implant can comprise a C-shaped sidewall that engages the lateral and anterior walls of the trachea, with extensions that engage a portion of the posterior wall without spanning the entire posterior wall. This provides some flexibility at the posterior aspect of the implant to allow for expansion at the posterior wall. As shown in FIG. 38, in some embodiments, the C-shaped sidewall engages only the anterior wall and the lateral walls. The sidewall can appose the entire length of the lateral sidewalls, or only a portion (as shown in FIG. 38). The sidewall can be formed of a back-and-forth minimalist wire design, or may have other constructions. In any case, the implant is configured to resist the radially inward force of a trachea that is prone to collapse. In some embodiments, to achieve the recoil strength required to resist airway collapse, the C-shaped wire form may be shape-set with a high over-size factor (i.e., in an unconstrained state the C-shape wire support frame has a diameter that is 1.1×-2× bigger than the native airway diameter).

In some embodiments, in the transverse perspective, the ends of the C-shaped sidewall may have a curvature with an inward bend to prevent a high pressure contact at the C-ends on the cartilage-trachealis junction. The region of the bends may have additional atraumatic features e.g., coating, soft covering layer (e.g. silicone), etc. This could be a high stress point or contact vulnerable tissue.

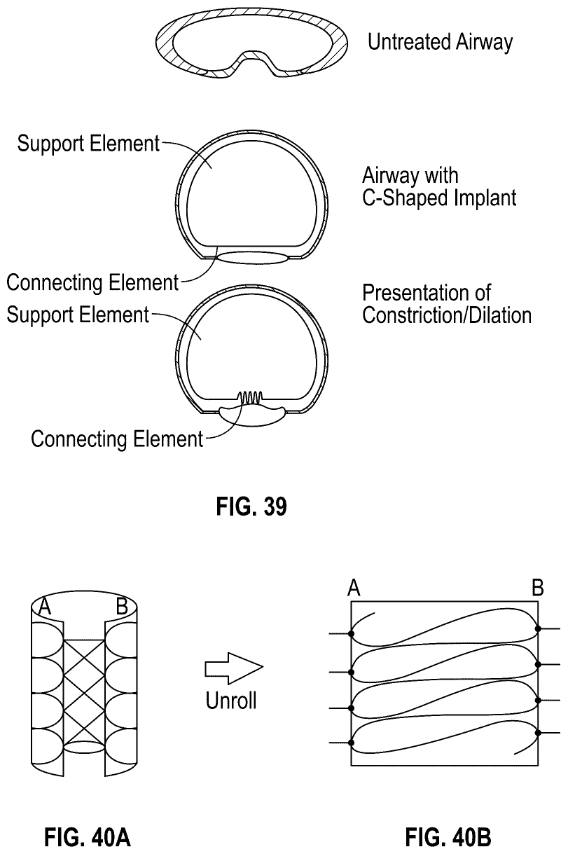

As shown in FIG. 39, in some embodiments the ends of the C-shaped sidewall can be connected to one another with a flexible and/or clastic tether (also referred to as a “connector” or “connecting element”). The tether can pull the ends toward one another so that C-shaped structure has a smaller diameter compared to its unconstrained state. This smaller diameter may be closer to the native airway diameter (e.g., slightly bigger so there is gentle apposition which may avoid tissue irritation due to friction), which may resolve the risk of tissue trauma, excessive granulation or moving through the tissue. It also preloads the resilient wire form structure which affects its ability to resist airway collapse.

The tether may be configured to resist tension applied by the C-ends, preventing them from moving apart more than a maximum distance, and allow the C-ends to move toward one another, optionally unencumbered. For example, in some embodiments the tether may comprise a thread (e.g., Kevlar, suture material, bioresorbable material, non-bioresorbable material, etc.) connecting the C-ends along the longitudinal dimension of the sidewall, similar to a shoelace (e.g., tether zigzagging from left to right, proximal to distal; optionally back from distal to proximal again, as shown in FIGS. 40A and 40B) or purse string (thread per level connecting left to right, each loop can be independently adjusted). The tether may cross over the trachealis muscle, which may prevent it from prolapsing into the airway. The tether may allow the implant diameter to be adjusted for the patient's airway, e.g., before implanting based on a sizer, or in situ based on performance. Moreover, the tether can allow the airway to keep its natural degrees of motion (e.g., dilation, constriction, elongation and shortening), which may further minimize tissue irritation and granulation formation or migration.

The tether across the free ends of the C-shape may be a different design based on the different needs. For example, EDAC may have different mechanics and needs than TBM. In some cases, EDAC can benefit from a wide ribbon running down the posterior trachealis muscle (e.g. silicone or polyurethane ribbon) while TBM may see more benefits from a shoe-string tether. Other examples are possible.

In some embodiments, tether may be configured to be adjusted over time, e.g., in an infant whose airway is growing, or to adjust the performance of the implant. During implantation the connection thread may pull the implant into a small OD that can be gradually increased by loosening the thread until a good fit is found. Additionally or alternatively, the tether may also be configured to easy removal. For example, the tether may be cut and removed from the wire form. This may also allow easy removal of the implant, as removal of tether enables removal of the single wire forming the sidewall of the C-shape.

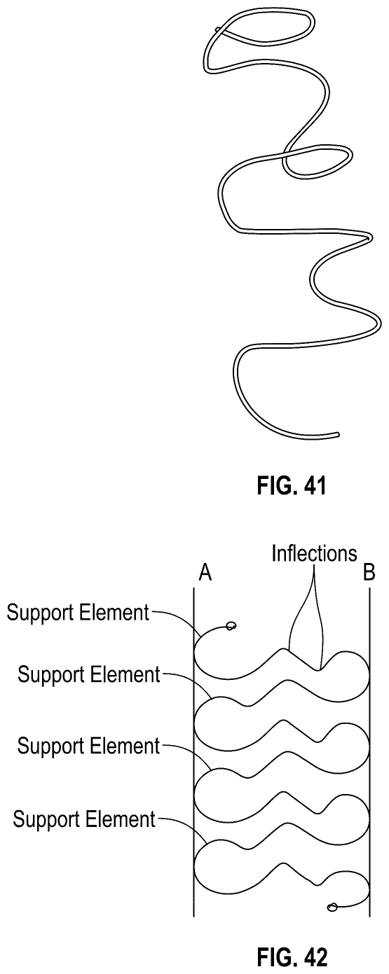

Variations on the C-shaped wire form can include additional curves, shark fin curves, radial bumps to engage between cartilage ribs to prevent migration. The back-and-forth wire pattern shown in FIGS. 40A and 40B may have additional bends or inflections, e.g. as shown in FIGS. 41 and 42. In some embodiments, for example as shown in FIG. 42, the wire form can have two inflections between each end of the C shape. The inflections may have a radius of curvature that has a low risk of fatigue failure (e.g., a ROC in a range of 2-5 mm). The additional inflections may facilitate radial expansion and contraction while in place or to radially contract for delivery. The additional inflections may create a wire pathway that crosses over the trachea's cartilage in a way that helps resist collapse (e.g., the wire may cross one row of cartilage more times with additional inflections). The additional inflections may improve anti-migration.

In any of the wire form designs herein, the ends of the wires can have a ball or loop to facilitate delivery or removal. The wire cross-section might be circle, semi-circle, oval, thin rectangle. The wire cross section can vary along length of the implant to impart different mechanical properties/functions. Likewise, the shape set implant cross-section can vary along length of the implant to impart different mechanical properties/functions.

The proximal and/or distal regions of a wire structure may be contained in an End Band (see “Concepts that may apply to any of the above ideas”)

Multiple C-shaped implant's may be implanted and joined with and End Band.

In some cases, a C-shaped implant may be implanted in the primary bronchi and another in the trachea and joined together with a Y-junction.

For EDAC, the C-shape may be inverted so the open part is anterior, which may help hold a prolapsing trachealis muscle out of the orifice.

The length of the implant may be short (e.g., a few cm), or as long as whole trachea, or trachea plus bronchi. The length to diameter ratio may be at least 2:1 for axial stability, e.g. OD of 20 mm and length of 40 mm. Multiple implants can be used with this minimum ratio (e.g. modular approach). In some cases, it may be beneficial to ensure the implants do not overlap or contact, e.g., place at least 1 diameter apart. The present technology can include a spacer to hold the implants relative to one another and prevent contact.

In some embodiments, the C-ends may be connected by a continuous strip and/or ribbon (e.g., silicone, polyurethane, etc.) running down the strip of trachealis muscle. Such a design may be less traumatic to the tissue along the strip as the pressure is more evenly distributed. Even though this design covers some of the inner lining it preserves most of the inner lining which may be enough for mucus removal. The strip can be flexible but with little elasticity to resist tension or have some elasticity to absorb forces.

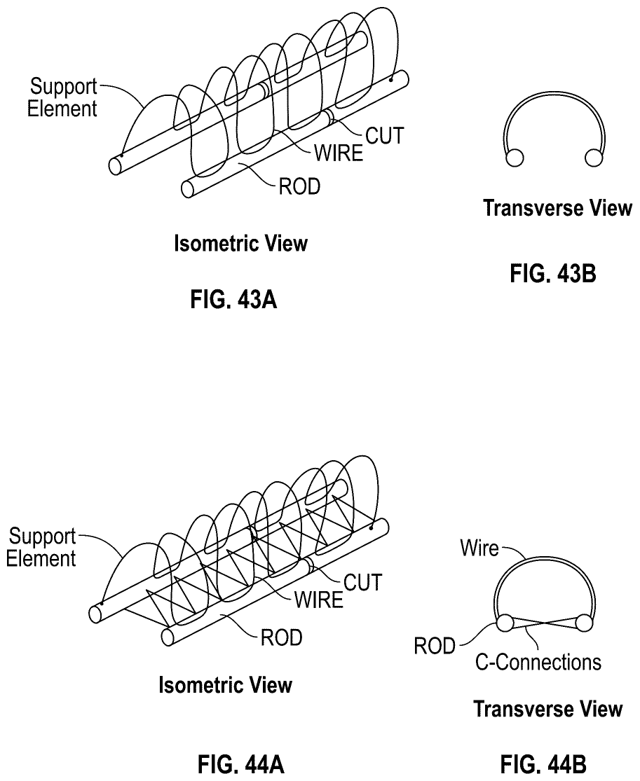

As shown in FIGS. 43A-43B and FIGS. 44A-44B), in some examples the implant can include two longitudinal flexible “rods” e.g., silicone or other soft, flexible, longitudinally stretchable, biocompatible material, that run down the cartilage/muscle junction on both sides of the C-shape to reduce traumatic forces that otherwise may be applied to the area where the trachealis muscle and tracheal cartilage meet. The rods can be used with a C-shape that is discontinuous (e.g., FIGS. 43A and 43B) or that has a tether (e.g., FIGS. 44A and 44B). The rods can hold the C-ends of the wire form and relative to one another. This feature may help to secure orientation, for example by nestling into a groove in this region. Also, if a tether were to fail the rods could prevent the wire from over opening at the ends. The wire structure can be embedded or connected to the rods at the C-ends. The rods may run the full length of the implant, or only a partial length. Optionally, rods may run the full length of an implant but with one or more cuts in the rods, which may allow the implant to elongate more freely. If the C-shaped implant has end bands or a Y-junction the side rods may be joined to or integrated with the End Bands or Y-junction

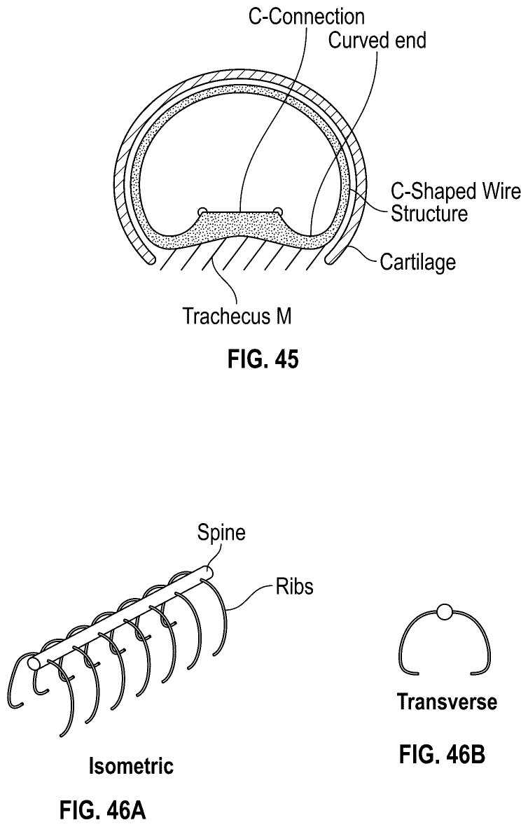

In some embodiments, where the tether meets the ends of the C-shape can be held off of the tissue surface by shape of C. For example, the C-ends can be curled radially inwardly so that the C-tether connections can be positioned off of or with less contact force on the trachealis muscle as shown in FIG. 45.

FIGS. 46A and 46B show a design having C-shaped wire support structures joined to a center spine. The spine may be an elongate ribbon or rod, (e.g. silicone or polymer, longitudinally stretchy/flexible) running the length of the implant and holding individual wire support “ribs”. Alternatively, the wire support structure may be a continuous wire winding back and forth, similar to FIGS. 40A and 40B, and others. Note, FIGS. 46A and 46B are shown with no C-connections but a “spine and ribs” design may or may not have C-connections.

In some embodiments, an implant having a spine and ribs may also have side rods. The combination of a center spine and two side rods may help to stabilize a wire structure, particularly if the wire structure is independent “ribs” as in FIGS. 47A and 47B. The C-connection shown is a silicone strip with perforations that enhance its ability to stretch longitudinally and provide some surface area of the native tissue to remain uncovered by the strip, which may facilitate mucous removal. Other C-connections such as sutures or no C-connection are possible. The spine in this figure is shown as having a flat cross section, like a ribbon or oblong shape, which may maximize airway lumen area.

The ribs may be each oriented in a plane orthogonal to the longitudinal axis of the trachea and the spine (and side rods) may be parallel to the axis.

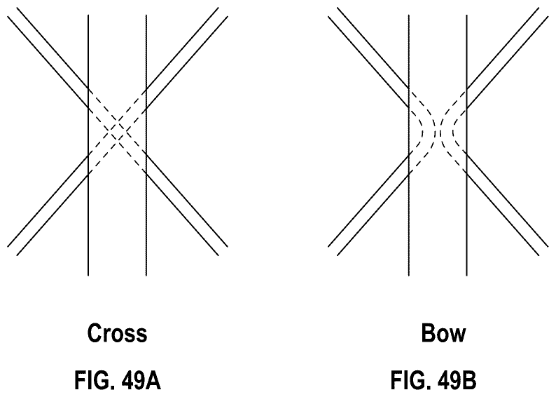

Wire support structures may be arranged similar to tent poles of a dome tent to apply radially outward force to the airway wall (see FIGS. 48A and 48B). The wires may include one or more (2 are shown) wire arcs oriented in an orthogonal plane to the axis, and one or more (2 are shown) wire arcs that are oriented in a plane that is acute to the axis. The acute wire arcs may cross one another (FIG. 49A) or have bow shapes and are held together at an intersection (FIG. 49B), optionally by a longitudinal support member, as shown.

Optionally the wire support structures may be connected to longitudinal support members (e.g., spine and side rods) that hold the wires in a desired orientation and distribute contact force. As shown in FIGS. 48A and 48B, the implant can include three longitudinal support members but less (e.g., 2 side rods) are possible.

The C-shaped implants of the present technology, while positioned within the proximal airway (such as the trachea or main bronchi), are configured to support a patency of the airway such that the airway appears substantially D-shaped during exhalation, as viewed on a fluoroscopic image showing a cross-section of the trachea. The implant is also configured to maintain the cross-sectional dimension of the lumen of the airway within a range of about 50% to about 100% of the maximum cross-sectional dimension of the lumen.

According to several embodiments, a cross-sectional shape of the implant can comprise a shape resembling the letter ‘D’ (referred to herein as “D-shaped”), thereby mirroring the shape of the native trachea and/or primary bronchi. A D-shaped implant can be configured to engage the full circumference of the trachea and/or primary bronchi, once implanted. For example, the D-shaped implant can comprise a plurality of support elements configured to engage and support the cartilage ribs of the trachea and a plurality of connecting elements configured to engage and support the trachealis muscle extending between the cartilage ribs. The support elements can be substantially C-shaped in a transverse cross-sectional view and one of the connecting elements can extend between an end of a first support element and an end of a second, adjacent support element. In some embodiments, the support elements have different properties than the connecting elements, which can enhance the ability of each element to resist collapse and permit natural movement of the trachea when the trachea applies forces to the implant.

The D-shaped implant can comprise an elongate member formed into a desired shape. For example, the elongate member can comprise a superelastic material and/or a shape memory material, such as Nitinol. The elongate member can be wound about one or more longitudinal axes to form one or more complete loops and/or partial loops. The D-shaped implant can comprise a single elongate member or multiple elongate members. In any case, the D-shaped implant can be configured to engage the trachea over a limited surface area, which can prevent or limit a granulation tissue formation response to the implant. Bends formed in the D-shaped implant via winding of the elongate member can have a large radius of curvature, to limit the stress and strain developed in these bends and thereby enhance a fatigue resistance of the D-shaped implant. The D-shaped implant may or may not include connecting struts extending between spaced apart portions of the elongate member. Additionally or alternatively, the D-shaped implant can include “shark-fin” bends along the winding path of the elongate member, which can provide more uniform support of the trachea, prevent pancaking, and/or prevent or limit the implant from migrating from its initial location within the trachea.

The support elements of the D-shaped implant can be configured to resist radially inward forces applied to the D-shaped implant by a collapsing trachea. The support elements can be configured to minimally contact radially opposing sides of the trachea. The support elements can have a high clastic modulus (e.g., as determined by gauge and/or Nitinol austenite finish (AF) temperature of the elongate member) to provide sufficient recoil to the trachea. The elongate member can have a shape configured to traverse the cartilaginous ribs across multiple locations, which may hold the cartilaginous ribs open while limiting or preventing pressure from being applied in weaker regions between the ribs. The elongate member can be formed to have a back-and-forth minimal wire pattern, which may allow the airway to elongate. The connecting elements can be configured to extend across the trachealis muscle and provide collapse resistance (e.g., by strengthening the support element, preventing trachealis protrusion, etc.) while allowing flexion (e.g., transverse constriction). In healthy patients, flexion of the trachea is primarily provided by deformation of the trachealis and longitudinal elongation of the trachea. The D-shaped implant may have unique advantages over other designs, such as, but not limited to, better accommodation of function of the esophagus (e.g., swallowing).

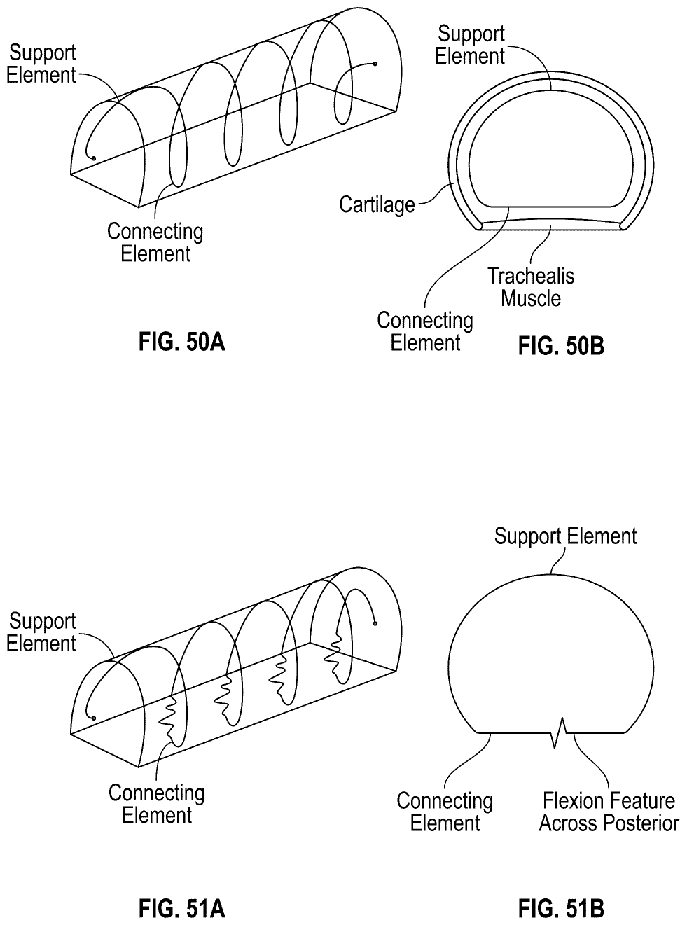

FIG. 50A is a perspective view of an example D-shaped implant, and FIG. 91B is a transverse cross-sectional view of the implant of FIG. 50A positioned within a trachea. According to various embodiments, the D-shaped implant can comprise an elongate member wound about a longitudinal axis to form a plurality of support elements and a plurality of connecting elements interposed between the support elements. In some embodiments, a proximal portion of the D-shaped implant and/or a distal portion of the D-shaped implant can have a lower stiffness and/or rigidity than an intermediate portion of the D-shaped implant. Accordingly, the proximal portion and/or distal portion can function as strain relief, so that forces applied to the trachea and/or deformation of the trachea gradually changes from regions of the trachea without the D-shaped implant to regions of the trachea with the D-shaped implant.

As shown in FIG. 50B, the support elements can be configured to be positioned proximate to and radially support the C-shaped cartilage ribs of the trachea while the connecting elements can be configured to be positioned proximate to and radially support the trachealis muscle extending between the ends of the C-shaped cartilage ribs. In some embodiments, one or more of the support elements can have a substantially C-shaped cross-sectional shape. As shown in FIG. 50B, the ends of each C-shaped support element can be substantially aligned with the locations at which the ends of each C-shaped cartilage rib connect to the trachealis muscle. As shown in FIG. 50A, the support elements can be spaced apart along a longitudinal dimension of the D-shaped implant. The connecting elements can extend at a diagonal angle to the longitudinal dimension of the D-shaped implant such that, when the D-shaped implant is positioned within the trachea, the connecting elements extend along a longitudinal dimension of the trachea, as well as extending circumferentially along the trachealis muscle. The angle at which the connecting elements extend relative to the longitudinal dimension of the D-shaped implant may be varied (e.g., made to approach orthogonal, made more acute, made more obtuse, etc.) to change one or more properties of the D-shaped implant. The connecting elements can be configured to provide resistance to collapse of the trachealis while allowing flexion (e.g., transverse constriction) and longitudinal elongation of the trachea. In some embodiments, the D-shaped implant includes additional curves, shark fin curves, and/or radial bumps to engage between cartilage ribs to prevent or limit migration of the D-shaped implant. Additionally or alternatively, a surface of the elongate member of the D-shaped implant can comprise micro-pillars and/or nano-pillars to prevent or limit migration of the D-shaped implant. Moreover, a thickness of the elongate member can be increased to provide greater resistance to migration of the D-shaped implant.

The properties of the support elements and the connecting elements can be tuned to the intended functions of the elements. For example, the connecting elements can comprise portions of the elongate member with a reduced wire gauge relative to the support elements so that the connecting elements are more flexible, less rigid, and conform more easily to the contour of the trachea, which can facilitate functioning of the esophagus. When the support elements and the connecting elements are formed form a single elongate member, the portions with a reduced wire gauge can be formed via etching, for example. In some embodiments, an elongate member with a reduced wire gauge can be employed at locations along the D-shaped implant where movement is required or desired, which can accomplish an appropriate longitudinal spring constant.

In some embodiments, joints (e.g., bends) between the support elements and the connecting elements are rounded to reduce peak strain or stress at the joints and improve a fatigue life of the D-shaped implant. One or both ends of an elongate member used to form the D-shaped implant can include a delivery feature (e.g., a ball, a loop, etc.) that is configured to facilitate delivery or removal of the D-shaped implant from the patient's anatomy. In some embodiments, the D-shaped implant can include one or more radiopaque markers, which can be used to assess function and/or position of the D-shaped implant radiographically.

According to various embodiments, the D-shaped implant can include a membrane (e.g., polymer, silicone, etc.) positioned across a portion of the D-shaped implant. The membrane can be configured to distribute forces applied to the trachea by the D-shaped implant. Additionally or alternatively, the member can be configured to facilitate the D-shaped implant maintaining a desired shape once positioned within the anatomy and in use. The membrane can be positioned along any suitable portion of the D-shaped implant. For example, the membrane can be positioned at an anterior side of the D-shaped implant (e.g., on the support elements). The membrane may have a micro-structured surface (e.g., Hoowaki, etc.). For example, an outer surface of the membrane can be micro-structured to prevent or limit migration of the D-shaped implant. The membrane may enhance lubricity on the inner surface to encourage mucus removal.

FIGS. 51A-53B illustrate additional examples of D-shaped implants. The features of the D-shaped implants of FIGS. 51A-53B can be generally similar to the features of the D-shaped implant of FIGS. 50A-50B. The discussion of the D-shaped implants of FIGS. 51A-94B will be limited to those features that differ from one another and from the D-shaped implant of FIGS. 50A-50B. Additionally, any of the features of the D-shaped implants of FIGS. 51A-94B can be combined with each other and/or with the features of the D-shaped implant of FIGS. 51A-51B.

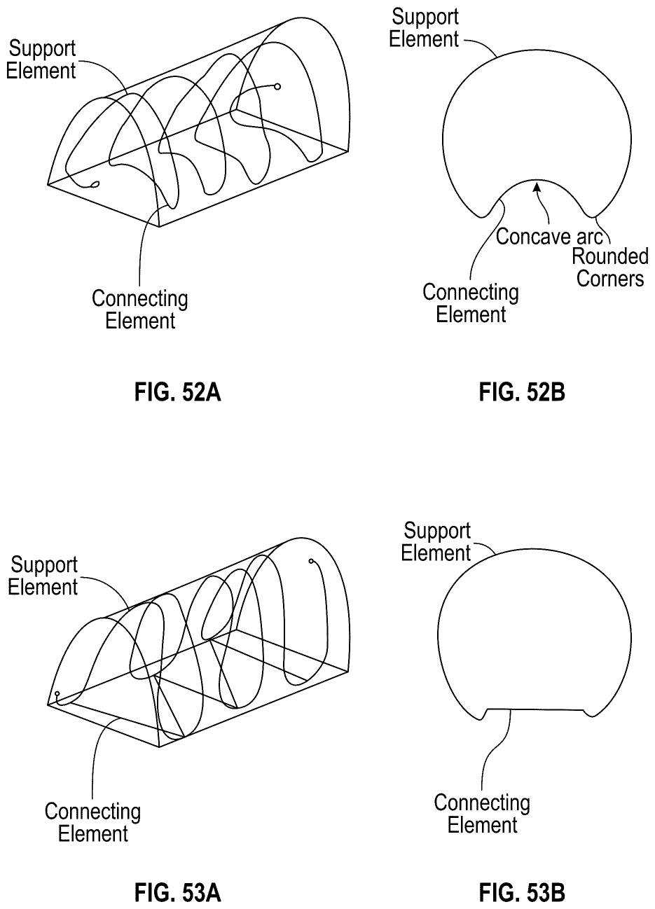

As shown in FIGS. 51A and 51B, in some embodiments, the connecting elements of the D-shaped implant may include bends that increase a flexibility of the connecting elements. Such bends can be formed in the sagittal plane, for example. Additionally or alternatively, as shown in FIGS. 52A and 52B, the connecting elements of the D-shaped implant may include bends in the transverse plane. Such transverse bends can provide room for the trachealis muscle and/or can modify a flexibility of the connecting elements.

The D-shaped implants shown in FIGS. 50A-52B comprise an elongate member that is wound about a longitudinal axis of the implant to form connecting elements integral with the support elements. However, in some embodiments, for example as shown in FIGS. 53A and 53B, the connecting elements can be formed separately from the support elements and secured to the support elements. The support elements can be formed by alternately winding an elongate member about a longitudinal axis of the D-shaped element and a secondary axis angled relative to the longitudinal axis. Accordingly, the support elements can comprise open (e.g., incomplete, etc.) loops that are angled relative to one another. Some of the support elements can extend circumferentially along the C-shaped cartilage ribs, while others of the support elements can extend longitudinally along a length of the trachea. Similar to the D-shaped implants of FIGS. 50A-52B, the connecting elements shown in FIGS. 53A and 53B can extend from one end at an apex, bend, or end of a first support element and an apex, bend, or end of a second, adjacent support element. The connecting elements can be configured to be positioned proximate to and extend across the trachealis muscle. The support elements and the connecting elements can comprise the same material or different materials. In some embodiments, both the support elements and the connecting elements comprise nitinol, for example.

The D-shaped implants of the present technology, while positioned within the proximal airway (such as the trachea or main bronchi), are configured to support a patency of the airway such that the airway appears substantially D-shaped during exhalation, as viewed on a fluoroscopic image showing a cross-section of the trachea. The implant is also configured to maintain the cross-sectional dimension of the lumen of the airway within a range of about 50% to about 100% of the maximum cross-sectional dimension of the lumen.

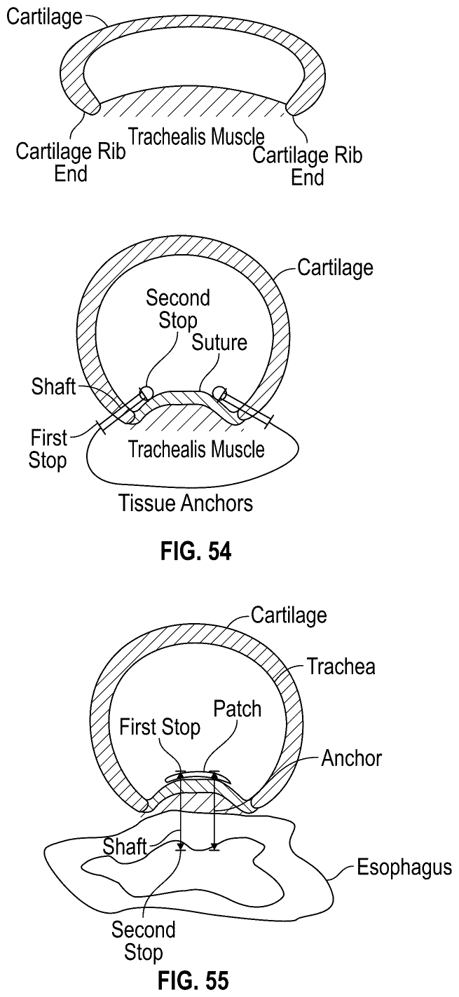

In some embodiments, a method of treatment of the present technology (e.g., a method of treating tracheobronchomalacia, etc.) comprises pulling the ends of C-shaped cartilage ribs toward one another to restore a more rounded and patent airway. As shown in FIG. 54 (top), an untreated trachea with compromised cartilage may be wide and narrow, with a large distance between the ends of the cartilage rib. As shown in FIG. 54 (bottom), a more patent trachea may be created by pulling the cartilage ends together and opening the tracheal lumen. Tissue anchors may be inserted into the cartilage ends and a suture may be tied to the anchors and tightened to bring the cartilage ends towards each other. Because the ends of the cartilage ribs comprise a strong material, the anchors may be inserted into the ends and traction applied through the ends with limited risk of tearing. In some embodiments, the tissue anchors may be inserted between adjacent cartilage ribs, which may require lower insertion forces. In any case, as shown in FIG. 54, the anchors may include a first stop (e.g., a flange, a T-post, etc.) that is configured to be positioned on an external side of the cartilage to engage the tissue. The anchors may include a second stop (e.g., a flange, a ball, a loop, etc.) that is configured to be positioned on the internal side of the cartilage. A shaft of the anchor can extend between the first and second stops of the anchor and can be configured to extend through the cartilage. The second stop can be configured to connect to a tether (e.g., a suture, an elastic band, etc.), which can be connected to a second stop of another anchor and tensioned to deform the cartilage. Multiple pairs of anchors, with each anchor of the pair connected to the other anchor of the pair by a tether, can be implanted along a length of the trachea or bronchi, for example every about 5 mm to every about 20 mm. In some embodiments, the pairs of anchors are not connected to one another, which can facilitate or permit longitudinal elongation of the airway. In some embodiments, one or more anchors includes a pharmaceutical agent configured to prevent or limit infection from occurring and/or spreading.

According to various embodiments, a method of treatment of the present technology (e.g., a method of treating excessive dynamic airway collapse, etc.) comprises anchoring the trachealis muscle of the trachea to the esophagus, for example as shown in FIG. 55. In some embodiments, the trachealis is anchored to the esophagus using an endotracheal approach. As shown in FIG. 55, in some embodiments tissue anchors may be deployed in the trachea and esophagus such that the anchors extend from within the trachea to within the esophagus. A tissue anchor can comprise a first stop configured to be positioned within the trachea, a second stop configured to be positioned within the esophagus, and a shaft extending between the first and second stops. The shaft can be rigid or flexible. In some embodiments, the shaft extends through the trachealis muscle when the anchor is implanted. A patch can be placed on the trachealis muscle before inserting the anchor so that the patch is positioned between the first stop and the trachcalis muscle. The patch can be configured to dispersedly distribute forces from the anchor to the tissue. Embodiments such as that shown in FIG. 55 may have particular benefit in an infant application to ensure and/or promote proper growth and/or proper spacing between trachea and esophagus.

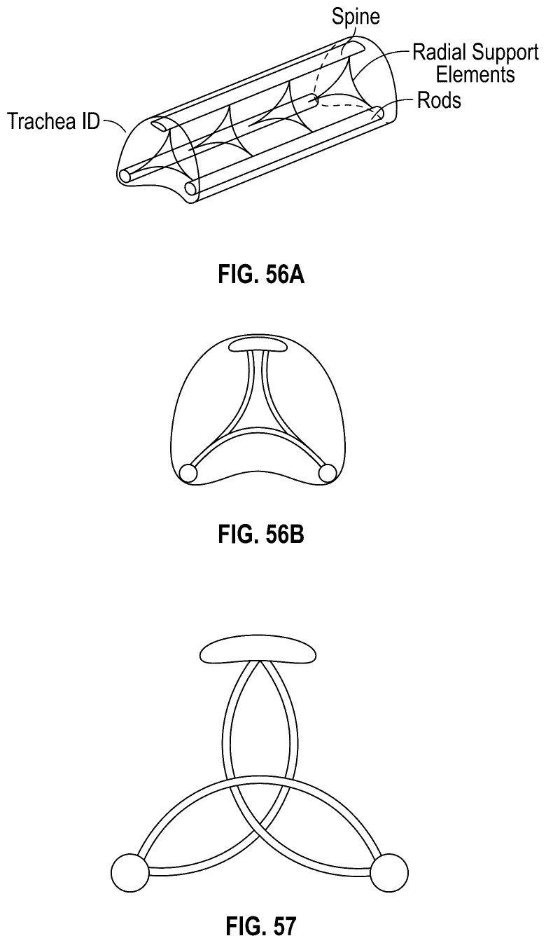

An implant may have longitudinal support elements that are configured to be positioned substantially parallel to a longitudinal axis of the airway. The longitudinal support elements can be configured to be pushed or supported radially outwardly to engage the airway. For example, the longitudinal support elements can be radially pushed or supported by radial support elements. FIGS. 56A and 56B illustrate an example implant with such longitudinal support elements and radial support elements in a perspective view and a transverse cross-sectional view, respectively. The longitudinal support elements may include a spine and two rods, which are spaced apart from one another about a circumference of the airway when the implant is deployed. Still, an implant of the present technology can include more than one spine and/or more than two rods. The spine and rods can be configured to be positioned proximate to and/or in contact with the luminal surface of the airway wall. In some embodiments, the spine has a smaller transverse cross-sectional dimension than the rods. For example, the center can be flattened relative to the rods. This small cross-sectional dimension can limit the spine from protruding into the lumen of the airway, and thereby enhance airway patency when the implant is deployed.

The radial support elements can have a specific shape to cause the radial support elements to apply radially outward force to the spine and rods, and thereby the airway walls. For example, as shown in FIG. 56A, the radial support elements can have a curvature that facilitates generating radially outward forces. In some embodiments, the radial support elements have a predetermined, expanded shape. When the implant is positioned within the patient's airway, the radial support elements can be deformed by the trachea. The radial support elements can comprise a superelastic material and/or a shape memory material, for example nitinol, such that the radial support elements tend to return to their predetermined shape after having been deformed. The support wires may be positioned and shaped to not contact the inner surface of the airway/trachea but instead reside within the airway lumen so that only the longitudinal supports contact the airway wall. This may maximize tissue surface exposure which may benefit the mucosal system and minimize a negative tissue response. The part of the wires that is connected to or embedded in the longitudinal supports may have an atraumatic feature such as a ball or bend or the wire may continue along the longitudinal support member. The device may be radially compressed to a smaller diameter for example during delivery through a delivery tube and may allow the airways/trachea to expand and contract radially. A compressed state is shown in FIG. 57, where the longitudinal supports are drawn toward one another, and the support wires bend and overlap

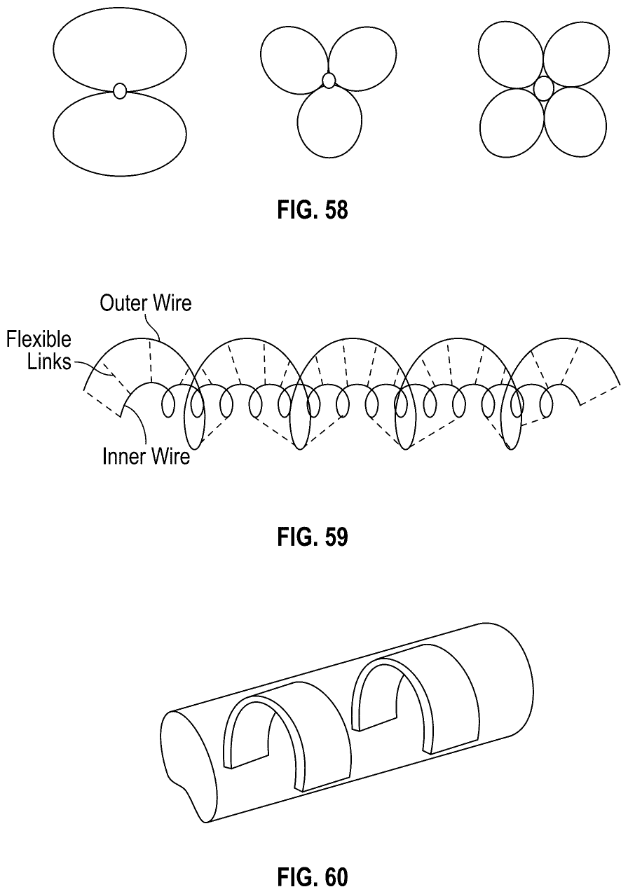

An alternative to the longitudinal support configuration is a spine that resides in a central longitudinal axis of the airway (i.e., in the middle of the airway and does not contact the walls) and wall-contacting wire supports that extend from the spine along the length of the spine. FIG. 58 shows cross-sectional views of different embodiments, wherein each wire support comprises a “petal”. The spine imparts mechanical strength to the implant, thereby allowing the petals to be especially minimal. Furthermore, tethering the spine to each loop or petal allows for controlled expansion (i.e., umbrella).

In the embodiment shown in FIG. 59, an implant design for treating TBM and EDAC comprises an outer wire and an inner wire connected by flexible links. The outer wire may comprise a larger diameter with minimal contact area with the tissue. The inner wire may not contact the tissue, and provides mechanical strength to the implant. The inner wire may comprise a smaller diameter than the outer wire. The flexible links may function like the cilia present in a natural trachea.

Some implants of the present technology include magnets (e.g., rare earth magnets) on opposing sides of the tubular implant and oriented to repel one another in order to apply a radially outward force to resist collapse. Pairs of opposing magnets may be longitudinally spaced and rotated in various orientations (e.g., 90 degrees). The tubular device may be a mesh or net or a sheet with openings. The openings may beneficially allow the tissue surface to be uncovered.

An arc-shaped patch may be implanted into an airway as shown in FIG. 60. The patch may have elastic properties that apply a radially outward force to the tissue. They may be shape-set with an arc shape that is larger than the lumen to provide apposition. Multiple patches may be implanted along the length of the trachea and/or bronchi. The patches may allow radial expansion, contraction, and elongation. Each patch may have a length (dimension along the longitudinal axis of the airway) in a range of 5 to 20 mm. The patches may be made with a polymer and Nitinol sheet or wires to give it favorable mechanical properties. The outer surface may be coated or treated to stick to the tissue. The inner surface may be lubricious to facilitate mucous removal.

Similar support structures to the embodiments herein comprising wires may comprise flexible tubes that are pressurized with fluid, wherein the hydrostatic pressure makes the tubes more rigid. This may allow a physician to adjust the degree of flexibility and rigidity based on how much fluid is injected into the frame. Over time, the hydrostatic pressure of the device may be adjusted by adding or removing fluid. The device may be removed from the patient by extracting fluid from the frame. The frame may have an inner lumen in fluid communication with a port. A user may insert a needle into the port to inject or retract fluid (e.g., saline).

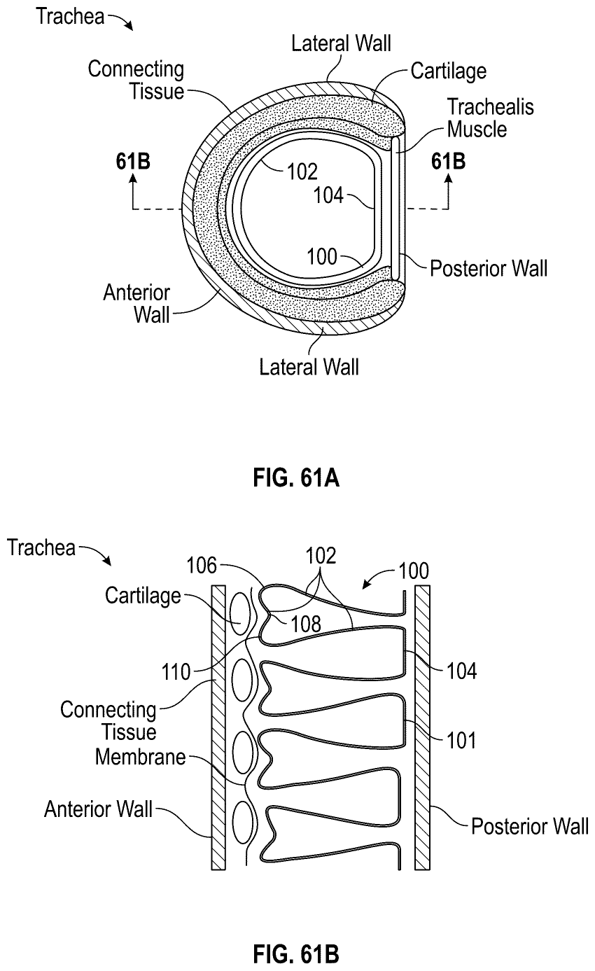

FIGS. 61A and 61B show an implant 100 configured in accordance with the present technology, implanted in a trachea or main stem bronchi. The implant 100 can comprise a generally tubular structure formed of a wire 101 wrapped around a longitudinal axis to form a series of loops, each comprising a 360 degree turn of the wire 101. Along a given loop, the implant 100 includes a C-shaped first portion 102 configured to be positioned at and engage the lateral and anterior walls of the trachea, (proximate the cartilage) and a second portion 104 configured to be positioned at and engage the posterior wall of the trachea (proximate the trachealis muscle). Each of the first portions 102 along some or all of the loops include a first length 106 of wire 101 configured to be positioned above a given cartilage ring, proximate and/or in contact with the membrane extending over and between adjacent cartilage rings. Each of the first portions 102 along some or all of the loops further include a second length 108 of wire 101 configured to be vertically aligned with the given cartilage ring. Each of the first portions 102 along some or all of the loops further include a third length 110 of wire 101 configured to be positioned below the given cartilage ring, proximate and/or in contact with the membrane extending over and between adjacent cartilage rings. The first and third lengths 106, 110 are thus configured to extend radially beyond the second length 108 such that the second length 108 is concave towards the cartilage ring while the first and third lengths are concave towards the trachea lumen. In some embodiments, all or a portion of the second lengths 108 can conform to the curvature of the luminal face of the cartilage rings. The first and third lengths 106, 110 thus help prevent migration while the bulk of the radial outward force exerted by the implant 100 on the trachea is distributed along the second length 108, thus leveraging the structural integrity of the cartilage to prevent erosion through the membrane.

With an implant designed specifically to be deployed such that the implant loops sit on the annular ligaments, those portions of the implant for deployment proximate the annular ligaments can be configured (e.g., by varying the shape, wire diameter, implant shape set diameter, material, etc.) such that the radial force exerted by the implant along those portions is optimal for the interaction with the annular ligaments. For example, the implant can have an optimized design/radial force configured to provide enough support during contraction (radial resistive force) to prevent collapse, but not excessive chronic outward force. Excessive chronic outward force on the softer annular ligaments could potentially result in overdilation or injury.

Likewise, the implant design can be fine-tuned such that those portions of the implant for deployment proximate the cartilage rings can be configured (e.g., by varying the shape, wire diameter, implant shape set diameter, material, etc.) to exert a radial force customized for the stronger/more supportive cartilaginous rings. This reduces the risk of injury or overdilation, and as such the design could have higher radial force which would be optimal for preventing collapse as well as migration.

In some embodiments, the implant can be delivered in an elongated form rather than a radially crimped form. FIG. 62 shows an example of an implant 100 being delivered to the trachea or main stem bronchi via a delivery system 200. In FIG. 62, the implant 100 is positioned in a delivery catheter 202 in an elongated configuration with a distal portion 102 of the implant 100 already released from the delivery catheter 202 and engaging the trachea wall. A proximal end 104 of the implant 100 remains coupled via a coupler 204 to the delivery system 200. A benefit of an elongated delivery configuration is that it enables partial deployment and, if needed, reloading back into the delivery catheter 202 mid-deployment. This feature can be useful, for example, for assessing initial deployment accuracy. If placement is suboptimal, the implant 100 can be retracted back into the delivery system and repositioned and redeployed.

### Example Diagnostic Systems and Methods

Rigid bronchoscopy and flexible bronchoscopy allow clinicians to perform diagnostic and therapeutic procedures of central airways (i.e. trachea, mainstem bronchi). For example, bronchoscopes can be used to deliver the implants of the present technology within the trachea. Flexible bronchoscopes have inner working channels of 2.0 mm (diagnostic scope) or 2.8 mm and 3.2 mm (therapeutic scope) and allow clinicians to access segmental airways; rigid bronchoscopes have inner working channels ranging from approximately 3 mm (pediatric) to approximately 8 mm (adult) and allow clinicians to access more central airways. A camera on the distal end of a flexible bronchoscope allows the clinician to visualize the airway on a display monitor; however, a rigid bronchoscope lacks that capability and visualization is accomplished by directly looking through the scope or insertion of a viewing telescope with light source. In accordance with the present technology, delivery, placement, and deployment of implants intended to treat central airway collapse can include visual features, such as bronchoscopically (i.e. pad printing) visible and/or fluoroscopically (i.e. platinum iridium, tantalum, stainless steel) visible markers on the delivery catheter. Additionally or alternatively, these features may be placed adjacent to the distal and proximal ends of the implant in its delivery profile to facilitate the positioning of the implant and delivery catheter relative to the anatomy and the bronchoscopic equipment. Bronchoscopically and fluoroscopically visible features are helpful for flexible bronchoscopy, while fluoroscopically visible features are helpful for rigid bronchoscopy.

Various embodiments of the present technology include one or more medical devices configured to be bronchoscopically delivered (manually or under robotic assistance) intraprocedurally to aid in deployment of an implant in a central airway (such as the trachea). In some embodiments, the medical device can be configured to determine the inner diameter of a central airway. Measurement can be accomplished by visual confirmation of device-to-airway engagement or digitally via optical coherence tomography (OCT), inspiratory and expiratory computerized tomography (CT), or virtual bronchoscopy. In some examples, visual confirmation includes inflating a non-compliant balloon (e.g., formed of nylon, nylon/Pebax, PET, etc.) of a known outer diameter to confirm physical contact under direct bronchoscopic visualization. Additionally or alternatively, visual confirmation can include inflating a compliant balloon (e.g., formed of silicone, polyurethane, elastomeric materials, etc.) with a known volume-to-diameter compliance curve. According to several embodiments, visual confirmation includes deploying a device that is normally collapsed in the delivery state to a device that self-expands to different diameters, such as shape-set superelastic and/or resilient wires or laser-cut frames. Yet another means of diameter acquisition may involve utilizing a probe with an automated articulatable distal tip coupled with a robotic system with contact, ultrasonic, laser, or pressure sensor, whereby detection of contact by controlled articulation suggests the inner diameter of the airway.

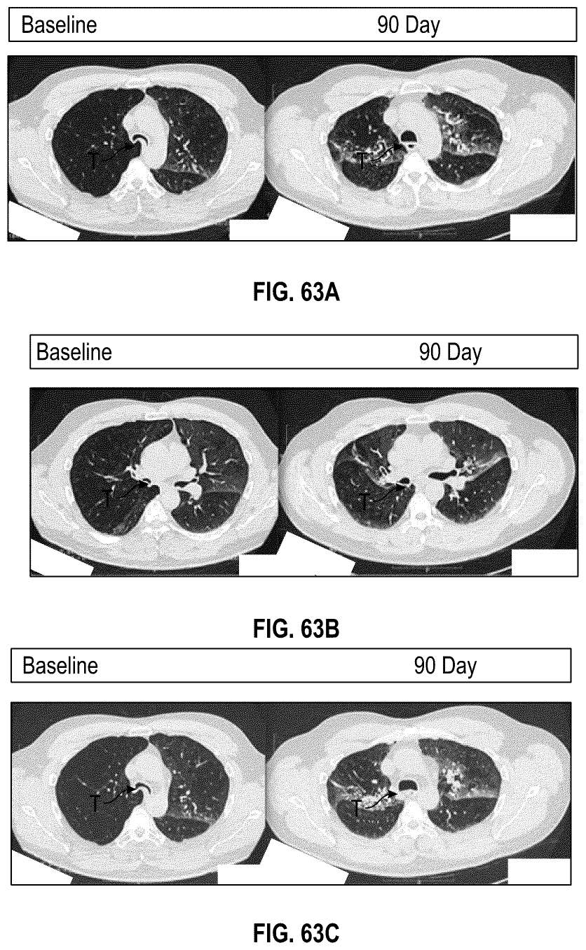

As previously mentioned, TBM and EDAC are characterized by the collapse of the central airways, namely trachea and the main bronchi. Both these pathologies often occur in patients who have other concomitant diseases such as emphysema and bronchitis. Patients with emphysema may be particularly vulnerable to central airway collapse. Emphysema results from lung tissue damage, which leads to air trapping in the lungs. Air trapped in the periphery of the lung may exert additional pressure on the central airways causing them to deform or collapse. Therefore, releasing the trapped air in emphysematous lungs using endobronchial implants positioned in the peripheral airways (for example, as described in U.S. Pat. No. 9,592,138, filed Sep. 13, 2015) may relieve the pressure on central airways and reverse the symptoms of ECAC (TBM and EDAC). FIGS. 63A-63C, for example, show CT scans of different patients' lungs before treatment and 90 days after treatment. As shown in the images on the left, the patients initially presented with ECAC, as evidenced by a collapsed trachea T (e.g., crescent shape) in the scan. After placement of the endobronchial implants in the peripheral airways, the central airway collapse was dramatically reversed, as evidenced by the open (e.g., D-shaped) trachea T in the scans.

The present technology includes improved methods for diagnosing ECAC. ECAC is typically diagnosed by direct bronchoscopic evaluation of central airway collapse during exhalation. The collapse of central airways can also be confirmed by comparing inspiratory and expiratory CT scans. Dynamic CT scans (i.e., CT scans during inhalation and exhalation) have also been used to confirm central airway collapse and diagnose ECAC. While CT scans provide quantitative information, the qualitative bronchoscopic evaluation remains the gold standard for diagnosing ECAC. These factors, combined with the fact that ECAC remains a highly underdiagnosed disease, underline the need for better ways of diagnosing ECAC.

Some methods of the present technology include receiving patient data including computed tomography (CT) data of a lung of the patient and generating a set of lung metrics by inputting the patient data into a machine learning (ML) algorithm. The set of lung metrics can be indicative of the degree, extent, and type of central airway disease. In some cases the ML algorithms can be specifically trained on central airway images, as opposed to the whole lung images that are typically used.

Another method for planning a treatment for a patient having a pulmonary disease comprises receiving patient data including scan data (e.g., computed tomography (CT) data, X-ray data, ultrasound data, CBCT data, QCT data, etc.) of a lung of the patient, and generating a set of lung metrics by inputting the patient data into an ML algorithm. The set of lung metrics can represent the degree, extent, and type of central airway disease. In some examples the ML algorithms can be specifically trained on central airway images, as opposed to the whole lung images that are typically used. Additionally or alternatively, the ML algorithm can be effective in population-level analyses, such that it can screen databases of lung images and identify patients at risk for central airway disease. For population-level analyses, the ML algorithm can also take digital health data (e.g., pulse oximetry data collected from smart watches) and standard pulmonary function testing (PFT)/other electronic medical record (EMR) data as inputs.

Another method for planning a treatment for a patient having a pulmonary disease comprises receiving patient data including audio recordings of the patient's chest and breathing (in addition to or instead of any of the inputs listed above), and generating a set of lung metrics by inputting the patient data into an ML algorithm. The set of lung metrics can be used to diagnose ECAC in individuals and populations.

Once a patient is diagnosed with ECAC, the standard procedure is to evaluate the patient's candidacy for tracheobronchoplasty. As part of the evaluation process, patients undergo a “stent trial,” where an airway stent is implanted in the central airways for a period of ˜1 week to confirm that reversing the central airway collapse will improve the patient's symptoms. The stents are not implanted for more than 1-2 weeks because of implant-related complications (foreign body reaction-related complications including granulation tissue, mucus, etc.). One of the unique features of the Apreo design is that the minimalistic design has the potential to minimize foreign body reaction. In addition, the Apreo design is made of a single wire, which extends when pulled and lends itself to be removed with relative ease. The dual benefits of i.) minimization of foreign body reaction-related complications and ii.) high removability makes the Apreo design ideal for applications as a short-term, medium-term, and long-term implant to keep central airways open.

The present technology includes a method of treating a patient with tracheobronchomalacia and/or excessive dynamic airway collapse, the method comprising positioning a device within a lumen of an airway of the patient such that at least a portion of the device engages a cartilaginous portion of a sidewall surrounding the lumen to prevent a cross-sectional dimension of the lumen from decreasing by more than 50% between expiration and inspiration. In some embodiments, the implant can be used for a “stent trial” to evaluate the patient's candidacy for tracheobronchoplasty. The implant will be placed in the patient for a period of 1-2 weeks and the changes in symptoms (e.g., dyspnea, PFTs) will be tracked. If the patient's symptoms show significant improvement over the course of the trial, the results will confirm the patient's candidacy for tracheobronchoplasty. In several of those cases where the implant is used for a “stent trial,” the implant is used as a challenge test before replacing it with a more permanent implant. The challenge test period can be used to inform the design and placement of the permanent implants. For example, challenge tests can be designed to test different sizes of implants, different implant positions, different implant mechanical properties etc. to identify the ideal configuration of an implant or a portfolio of implants placed in series that can be used for a permanent solution for ECAC.

In the foregoing examples, the implant can be replaced on a regular basis (e.g., after every 6-mo). This strategy may allow for reducing the risk of implant complications further. In certain examples, the implant can be replaced on a regular basis (e.g., after every 6-mo) until implant-induced fibrosis results in sufficient tissue strength to prevent airway collapse. This strategy may allow for reducing the risk of implant complications further and eliminate the need for a long-term implant.

The present technology further includes a method of treating a patient with tracheobronchomalacia and/or excessive dynamic airway collapse, the method comprising: drug-based approaches (e.g., warfarin) to induce calcification of airways, which may result in restoring the strength of airway tissue thereby reversing central airway collapse. The calcification-inducing drugs can be delivered as a coating on the implant or administered through local injections or systemic administration in conjunction with implants. (See Nour S A, Nour H A, Mehta J, Roy T, Byrd R. Tracheobronchial calcification due to warfarin therapy. Am J Respir Crit Care Med. 2014 Jun. 15; 189 (12): e73. doi: 10.1164/rccm.201305-0975IM. PMID: 24930540.)

Several embodiments of the present technology include a method of treating a patient with tracheobronchomalacia and/or excessive dynamic airway collapse that includes implant design, placement, and/or follow-up evaluation achieved via the assistance of robotically-assisted bronchoscopy. In some cases, the robotic bronchoscopy helps to combine the diagnosis of ECAC and imaging/measurement of airways for implant sizing into one procedure. In certain examples, robotic bronchoscopy aids in additional imaging (e.g., of major vessels, the esophagus) etc. that may help in better positioning of the implant. According to some embodiments, robotic bronchoscopy aids in better airway measurements that can inform implant sizing (e.g., the robotic arm can adjust its length to accommodate changes due to breathing, similar to naval “station keeping”). In any of the embodiments disclosed herein, the implant diameter and/or wire gauge is configured to accommodate an infant trachea. By way of background, the neonatal trachea is about 3-4.5 mm in diameter, 20-30 mm in length, and slightly larger, ˜1 mm, at the top. It elongates about 46% during inhalation. In contrast, the adult trachea is about 18 mm in diameter, 100-120 mm in length, and elongates ˜20% during inhalation. Some implants of the present technology can be shape-set with an unconstrained shape having an outer diameter that is in a range of 1.1 to 2 times the native trachea diameter (or having a circumference that is 1.1 to 2 times the native circumference) may be selected for the patient. (e.g., an infant sized O-shaped implant may have an outer diameter in a range of 3.3 mm to 9 mm). Oversizing the outer diameter in the current design can help clear mucous to keep airway patent but also contributes to radial force and secure apposition provided by the implant. Since the infant trachea is small a method of treatment may involve selecting an implant that is well oversized to keep the trachea patent from mucous buildup. Additionally or alternatively, the implant can have a longitudinal spring constant that approximates the longitudinal elongation of an infant trachea (about 46% for an infant).

The implants of the present technology can also be configured to accommodate growth of the infant trachea (diameter and length) over time. For example, the sidewalls of the implant can have a shape that expands in multiple directions. In some cases, the implant is configured to be incrementally expanded periodically (for example, by expanding a balloon within the implant) to match the expanding airway diameter. In some embodiments, the implant is oversized to a greater extent than would an adult implant.

An implant may not need to be oversized as much in an adult TBM treatment compared to an infant treatment. A method of treatment may involve selecting an implant that is oversized to provide secure apposition and collapse resistance. The implant can be shape-set with an unconstrained shape having an outer diameter that is in a range of 1.1 to 1.5 times the native trachea diameter (or having a circumference that is 1.1 to 1.5 times the native circumference). An adult sized O-shaped implant, for example, may have an unconstrained outer diameter in a range of 19.5 mm to 36 mm, and unconstrained length in a range of 30 mm to 120 mm, and/or a longitudinal spring constant of matching adult elongation (e.g., 20% for an adult).

In any of the foregoing embodiments, the proximal and/or distal section of the implant may be less stiff (as compared to the remainder of the implant) to avoid a drastic change in mechanics (e.g., a mechanical mismatch of the implant versus the native trachea) from the native to reinforced airway (like a strain relief) which may reduce tissue response. This can be done by narrowing (e.g., tapering) the core wire in these regions or changing the pitch.

Any of the implants can have one or more anti-migration features, including higher contact density at proximal end, flaring of the outer diameter at or near the proximal end, flaring out some bends (apexes) so they engage in surface contour (e.g., cartilage rings), etc.

Any of the implants shown herein may have a biodegradable coating or members that initially add rigidity and over time dissolve and decrease rigidity. In some embodiments, the implant can be entirely biodegradable, which may be particularly helpful in treating infants, as such a feature does not need to be removed or adjusted as the child grows.

The proximal and/or distal regions of the implant may be less rigid that the intermediate section to act as a strain relief so the change in forces applied or movement of tissue from an area without an implant to the area with an implant is gradual (i.e. a mechanical mismatch is gradual). Proximal and distal regions may be gradually more flexible to act like strain reliefs. This can be done by narrowing (e.g., tapering) the core wire in these regions or changing the pitch.

### CONCLUSION

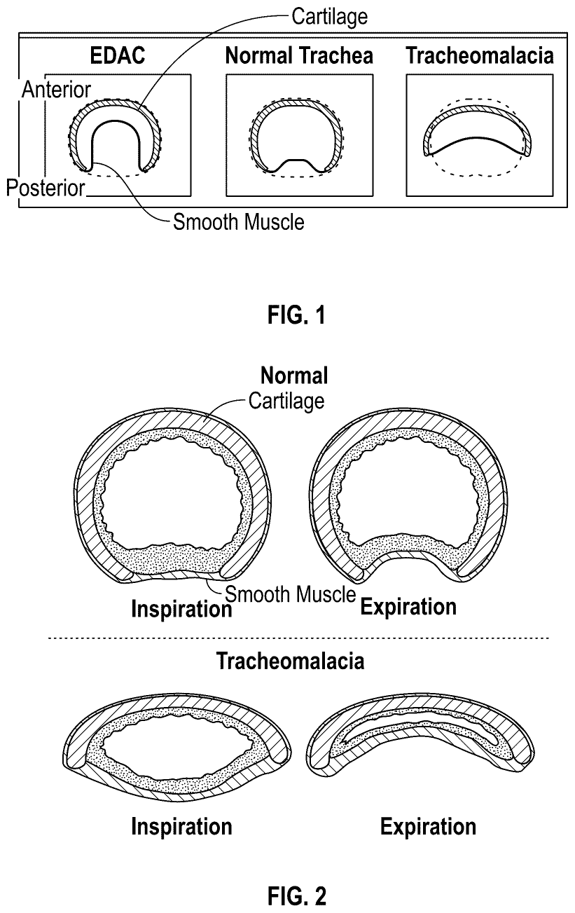

Although many of the embodiments are described above with respect to systems, devices, and methods for ECAC, the technology is applicable to other applications and/or other approaches. For example, the present technology may be used for treating other pulmonary conditions, such as chronic obstructive pulmonary disease (COPD) (such as emphysema). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-63C.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An implant for treating excessive central airway collapse (ECAC) of an airway, the implant comprising:
a wire-form implant configured to be positioned within the airway, wherein the implant comprises a C-shaped sidewall, and wherein the implant is configured to be positioned within the airway such that the C-shaped sidewall is positioned proximate a cartilaginous portion of the airway and presses outwardly against the cartilaginous portion.

2. The implant of claim 1, wherein, while positioned within the airway, the implant is configured to support a patency of the airway such that the airway appears substantially D-shaped during exhalation, as viewed on a fluoroscopic image showing a cross-section of the trachea.

3. (canceled)

4. The implant of claim 1, wherein the implant is configured to allow the airway to longitudinally lengthen and shorten while the device is positioned within the lumen.

5. The implant of claim 1, wherein the implant is configured to prevent contraction and expansion of the cartilaginous section.

6. The implant of claim 1, further comprising a tether extending between the free ends of the C-shaped sidewall.

7. The implant of claim 1, wherein the C-shaped sidewall is formed of a wire having a serpentine path such that the wire extends around a cartilage ring in a first circumferential direction, then turns and extends around the same or an adjacent cartilage ring in a second circumferential direction opposite the first circumferential direction.

8. The implant of claim 1, wherein the implant prevents a cross-sectional dimension of the airway lumen from decreasing to less than 50% of a maximum cross-sectional dimension of the lumen.

9. The implant of claim 1, wherein the airway is a trachea.

10. The implant of claim 1, wherein the airway is a primary bronchus.

11. The implant of claim 1, wherein the implant has a proximal portion, a distal portion, and an intermediate portion therebetween.

12. The implant of claim 11, wherein the proximal portion is more flexible than the intermediate portion.

13. The implant of claim 11, wherein the distal portion is more flexible than the intermediate portion.

14. The implant of claim 1, wherein a surface area of the portion of the implant engaging the cartilaginous portion of the airway sidewall is less than 10% of a total surface area of the airway sidewall.

15. The implant of claim 1, wherein a surface area of a portion of the implant engaging the sidewall of the airway is less than 10% of a total surface area of the sidewall along the length of the sidewall spanned by the implant.

16. The implant of claim 1, wherein the implant is formed from a single wire.

17. The implant of claim 16, wherein the wire forms a plurality of bends, each bend of the plurality of bends having a radius of curvature no less than about 2.0 millimeters.

18. The implant of claim 1, wherein the implant includes a tether extending across the free ends of the C-shaped sidewall, and wherein the tether is configured to be positioned proximate the muscular portion of the airway sidewall when the implant is implanted.

19. The implant of claim 1, wherein a surface area of a portion of the implant engaging the sidewall of the airway is less than 5% of a total surface area of the sidewall along the length of the sidewall spanned by the implant.

20. The implant of claim 1, wherein the implant has a crossing profile of at least one of 10 mm, 15 mm, 20 mm, 25 mm and 30 mm.

21. An implant system comprising the implant of claim 1, wherein the wire-form implant is a first wire-form implant configured to be positioned within a trachea of a patient, wherein the implant system further comprises:
a second wire-form implant configured to be positioned within a bronchus of the patient, wherein the second wire-form implant is configured to be positioned within a sidewall of the bronchus;
wherein a surface area of a portion of the first wire-form implant engaging the sidewall of the trachea is less than 10% of a total surface area of the sidewall along the length of the sidewall spanned by the first wire-form implant and a surface area portion of the second wire-form implant engaging the sidewall of the bronchus is less than 10% of a total surface area of the sidewall along the length of the sidewall spanned by the second wire-form implant.

22. The implant system of claim 21, wherein the first wire-form implant comprises a cross-sectional shape that is O-shaped, C-shaped or D-shaped and the second wire-form implant comprises a cross-sectional shape that is O-shaped.
